SEQUENCE LISTING (110 sequences)

SEQ ID NO: 1 - DNA, 381 bp, Lama glama
atggcccaggtgcagctgcaggagtctgggggaggcttggtgcaggctggggggtctctacacctctcctgtacagcctctggctctgagaacatgttgcgttggtatgccgtcggctggtaccgccaggctccagggaagcagcgcgagttggtcgcaactattactcgtggtggtagtacaaacgttgtagactccgtgaagggccgattcaccatctccagagataatgccaagaatatggtggcgctgcaaatgaacagcctgaaacctgaggacacagccgtctattcctgtgcagcagcgtatgcgccttatactgactatcatcccgtagaatatgtctactggggccaggggacccaggtcaccgtctcctca

SEQ ID NO: 2 - DNA, 378 bp, Lama glama
caggtgcagctgcaggagtctgggggaggattggtgcaggctggggactctctgagactctcctgtgcagcctctggacgcaccttcgatacccatgacataggctggttccgccaggctccagggaaggagcgtgagtttgtagcagctatggcttggagtggtggtagcacatactatgcagactctgtgaagggccgattcaccatctccagagacagcgccaagaacacggtcaatcttcaaatgaacagcctgaaacctgaggacacggccgtttattactgtgcatcagaagcgttccagtcctctaggctgtatagcgactatcgtcggtatcactactggggccaggggacccaggtcaccgtctcctca

SEQ ID NO: 3 - DNA, 354 bp, Lama glama
caggtgcagctgcaggagtctggaggaggcttggtgcagcctggggggtctctgagactctcctgtgcagcctctggaagcggcttcagtctcagagtcatgggctggtaccgccaggctccagggaaggagcaccgcttggtcgcaactattagtaggactggtatcacaaattatgcagactccgtgaagggccgattcaccatctccagagacaacgccaaggacacggtgtatctgcaaatgaacaacctgaaacctgaagacacggacgtctattactgctatgcacggaatttcgtaggcgatgactttaatctctggggccaggggacccaggtcaccgtctcctca

SEQ ID NO: 4 - DNA, 351 bp, Lama glama
caggtgcagctgcaggagtctgggggaggcttggtgcagcctggggggtctctgagactctcctgtgcagcctctggaagtatctccgatatgagagccatgaactggtaccgccagcctccagggaaggagcgcgagttggtcgcgcaaatatttctcactggtactatttactatgcggactccgtgaagggccgatttaccatctccaaggacaacgccaagaacacggtatatctgcaaatgaacaacctgaaactggaggacacggccgtctatagctgtagtgcagaccacttcctcgatcagtttgattcttggggtcaggggacccaggtcaccgtctcctca

SEQ ID NO: 5 - DNA, 369 bp, Lama glama
caggtgcagctgcaggagtctggaggaggattggtgcaggctgggggctctctgagactctcctgtgcagcctctggacgcaccttgtacgccatgggctggttccgccaggctccagggaaggagcgtgagtttgtagcagctaaaagctggaatggtggtaacacatactatgcagactccgtgaagggccgattcaccatctccaaagacaacgccaagaacacggtgtatctgcaaatgaacagcctgaaacctgaggacacggccgtttataattgtgcagcttcgtcctgggactcgcgtggtagtagtaattacgttcattatgactactggggccaggggacccaggtcaccgtctcctca

SEQ ID NO: 6 - DNA, 354 bp, Lama glama
caggtgcagctgcaggagtctggaggaggcttggtacagcctggggggtctctgagactctcctgtgcagcctctacccgcatcatcttcggtgtcaatgccatgggctggtaccgccaggctccagggaaggagcgcgagttggtcgcacgtattgaggatggtggtaccacaaactatgcagactccgtgaagggccgattcaccatctccagagacaacgccaagaacacggtgtacctgcaaatgaacagcctgaaacctgaggacacgggcatctattactgcaattaccacaactcttactacggctcggactactggggcaaagggacccaggtcaccgtctcctca

SEQ ID NO: 7 - DNA, 372 bp, Lama glama
caggtgcagctgcaggagtctgggggaggattggtgcaggctgggggctctctgagactctcctgtgcagcctctggacgcaccttcagtagatccgccgtggcctggttccgccaggctccagggaaggagcgtgaatttgtcgcaggtattagctggagtggtcgtaacgtaaactatgcaaactccgtgaagggccgattcaccatctccagagacagtaccaagaacacggtgtatctgcaaatgaacagcctgaagcctgaggacacggccgtttattactgtgcaaccagccaattcgggagtggtaccagcgactcggaacagtatgactactggggccaggggacccaggtcaccgtctcctca

SEQ ID NO: 8 - DNA, 363 bp, Lama glama
caggtgcagctgcaggagtctggaggagggtcggtgcagcctggggggtctctgagactctcctgtgcagtctctagaagccgcttcagtctcgtgaccatgggctggtaccgccaggctccagggaaggagcgcgaattggtcgcggctatcgcaattggtggcagcactaaatatgcagactccgtgaagggccggttcaccatctccagagacaacgccaagagtacggtgtatcttcaaatgaacagtctacagcctgaggacacggccgtctattattgtaacgaagcgcgagtgtacgacagtaactggtacccaagggactattggggccaggggacccaggtcaccgtctcctca

SEQ ID NO: 9 - DNA, 375 bp, Lama glama
caggtgcagctgcaggagtctggaggaggattggtgcaggctgggggctctctgagactctcctgtgcagcctctggacgcaccagcgatatctatgccatggcctggttccgccagactccagggaaggaccgtgagtttgtagcagctattacatggaatgatggtctcacatactatgcaggctccgtgaggggccgattcaccatctccagagattacgccaagaacacggtgtatctacaaatgaacaacctgaaacctgaggacacggccgtttattacggtgcagtgacacgccgtagatttgttacactccgcgtccctgatgactatgacaagtggggccaggggacccaggtcaccgtctcctca

SEQ ID NO: 10 - DNA, 375 bp, Lama glama
caggtgcagctgcaggagtctgggggaggattggtgcaggctggagactctctgagactctcctgcgcagcctctggattccccttcagtagctcggccatgggctggttccgccagccaccaggaaaggagcgtgagtttgtaacaagtattaactggagtggtgttcatacacgccatgcagactccgtgaagggccgattcaccatctccagagacaacgccaagaacacggtgtatctgcaaatgaacagcctgaaacctgaggacacggccgtttattactgtgcaacagatccgggaaactttggactgcctaacagtgaattagagtttaactactggggccaggggacccaggtcaccgtctcctca

SEQ ID NO: 11 - DNA, 369 bp, Lama glama
  misc_feature (80)..(80): n is a, c, g, or t
caggtgcagctgcaggagtctggaggaggcttggtgcaacctgggaggtctctgagactctcctgtgcagcctctggaantatcttcagtagctataccatcatgggctggttccgccaggctccagggaaggagcgtgagtttgtagcatccattagctggagtggcggtaacatatactatgcagactccgtgaagggccgattcaccgtctccagagacgacgccaagaacacggtgtatctggatatgaacaatccgaaacctgaggacacggccgtttattactgtgcatcagacctccgaatgtacgaaggtgactggtacggaggctactggggccaggggacccaggtcaccgtctcctca

SEQ ID NO: 12 - DNA, 372 bp, Lama glama
caggtgcagctgcaggagtctgggggaggattggtgcaggctgggggctctctgagactctcctgtgcagcctctgaacgcacctctggaatgcatgccacggcctggttccgccaggctccagggaaggagcgtgagtttgtagcacgaattttccacagtggtccttacacatactatgcagactccgtgaagggccgattcaccatcttcagagacagcgccaagaacacggtggatctgcaaatgaacagcctgaaatctgaggacacggccgtttattactgtgcagtcgctcggtcatggagcgatctggacttcgtagacgggtatgactactggggtcaggggacccaggtcaccgtctcctca

SEQ ID NO: 13 - DNA, 351 bp, Lama glama
caggtgcagctgcaggagtctgggggaggactggtgcagcctggggggtctctgagactctcctgtgcagcctctacaagcgtcttcggtgtcaatgacatgggctggtaccgccaggctccagaaaaggagcgcgagttggtcgcacgtatatcggatggtggtaccacaaactatgcagactccgtgaagggccgattcaccatctccagagacaacgccaagaacacggtgtatctgcaaatgaacaacctgaaagctgaggacacggccatctattactgcaattaccacaactattactacggcctggacttctggggcaaaggcacccaggtcaccgtctcctca

SEQ ID NO: 14 - DNA, 387 bp, Lama glama
caggtgcagctgcaggagtctggaggaggattggtgcaggctgggggctctctgagactctcctgtgcagcctctggacgcaccttcagtagcactgccatggcctggttccgccaggctccaggacaggagcgtgagtttgtagcaactattagctggagtggtgataacacaaaatatgcagactccgtgaagggccgattcaccatctccagagacaacgccaagaacacggtgtatctgcaaatgaacagcctgaaacctgaggacacggccgtttattactgtgcagcagaccccgggaactttggggtacccatcgagtattttatgtttgactactggggccaggggacccaggtcaccgtctcctcagcggccgcatac

SEQ ID NO: 15 - DNA, 363 bp, Lama glama
caggtgcagctgcaggagtctgggggaggattggtgcagcctggggggtctctgagactctcctgtgtagcctctggaagcatcggtagtatcaaccacatggcctggtttcgcaagcctccagggaaggagcgcgagttggtcgcatcaattactctggctccggatgaaatcgcctactatgcagactccgtgaagggccgattcgccatctccaaaggcaacgcccagcccaccttgtatctgcaaatgaacagcctgaaacctgaggacacggccgtctactattgtcattttcgtcagatccgtgcgaacgcacatgatgtctactggggccaggggacccaggtcaccgtctcctca

SEQ ID NO: 16 - DNA, 378 bp, Lama glama
caggtgcagctgcaggagtctggaggaggattggtgcaggctgggggctctctgagactctcctgtgcagcctctggacgcaccgtcagttatcgtgtgatgggctggttccgccaggctccagggaaggagcgtgagtttgtagcgttcgtggactggagttcaggtactgcatactatgcagagtccgtgcagggccgattcatcatgtccagagacactgccgacaacacggggtttctgcaaatgaacaacctgaaacctgaggacacggccgtttattactgcgcagcagatcctttttttcgagtcatcgcttatagcgaccctaaacgctatagctactggggccaggggacccaggtcaccgtctcctca

SEQ ID NO: 17 - DNA, 369 bp, Lama glama
caggtgcagctgcaggagtctggaggaggattggtgcaggctgggggctctctgagactctcctgtgtagcctccggacgcaccttgtacgccatgggctggttccgccaggctccagggaaggagcgtgaatttgtagcagctactagctggaatggtggtaatactaactatggagagtccgtgaagggccgattcaccatctccaaagacaacgccaagaacacggtgtatctgcaaatgaacagcctgaaacctgaagacacggccgtttataattgtgcagcttcgtcccgggactcgcgtggtacaagtacttacgccagttatgactattggggccaggggacccaggtcaccgtctcctca

SEQ ID NO: 18 - DNA, 381 bp, Lama glama
caggtgcagctgcaggagtctgggggaggattggtgcaggctggggactctctgagactctcctgtgcagcctctggacgcaccttcagtagcctttccgcgggctggttccgccaggctccagggaaggagcgtgagtttgtagcagctattaggtggagtgattataacacatactatgtagactccgtgaagggccgattcaccatctccagagacaacgccaagaacacggtgtatctgcaaatggacagcctgaaacccgaggacacggccgtttattactgtgcagccacggcattgggataccggtacgcctccctggagtcgcggaatgagtatcgctattggggccaggggacccaggtcaccgtctcctca

SEQ ID NO: 19 - DNA, 375 bp, Lama glama
caggtgcagctgcaggagtctggaggaggattggtgcaggctgggggctctctgagactctcctgtgcagcctctggacgcacctccgatatctatgccatggcctggttccgccaggctccagggaaggaccgtgagtttgtagctgctattacatggaataatggtgaagtatactatagcaactccgtgaagggccgattcaccatctccagagattacgccaagaacacggtgtatctacaaatgaacagcctagaacctgaggacacggccgtttattacggtgcagccacacgccgtagataccatacactccgcgtctctggtgattatgacaactggggccaggggacccaggtcaccgtctcctca

SEQ ID NO: 20 - DNA, 363 bp, Lama glama
caggtgcagctgcaggagtctggaggaggcttggtgcagcctggggggtctctgagactctcctgtgtagcctccagaagcatcggcagtatcaaccacatggcctggtatcgcaaggctccagggaaggagcgcgagttggtcgcgtcaattactcttagtcccagtgagaccgtgtactatacagaagtcgtgaagggccgatttgccatctccaaagggaacgcccagaataccttgtatctgcaaatgaacagtctgaaacctgaggacacggccgtctactattgtcattttcgtcagattcgtgcgatcgcggatgatgtctactggggccaggggacccaggtcaccgtctcctca

SEQ ID NO: 21 - DNA, 366 bp, Lama glama
caggtgcagctgcaggagtctggaggaggcttggtgcagcctgggggttctctgagtctctcctgtacaacttctggagagtaccacactattaattacatgggatggtaccgccaggctccagggaaggagcgcgagttagtcgcacgtattagtttcggtggtcgcatagaatatggagacttcgtgaagggccgattcaccctctccagaaacaacgccaagaacacggtgaatctacaaatgaacagcctgaagcctgaggacacggccgtctattactgtaatggagaaattactacactgattagtgtcttaagtaccgaccagtactggggccaggggacccaggtcaccgtctcctca

SEQ ID NO: 22 - DNA, 351 bp, Lama glama
caggtgcagctgcaggagtctgggggaggcttggtgcagcctggggggtctctgagactctcctgtgcagcctctggaagtatctccgatatgagagccatgaactggtaccgccagcctccaggaaaggagagcgagctggtcgcgcaaatatttctcactggtacgatttactatcgggactccgtgaagggccgatttaccatctccaaggacaacgccaagaacacggtatatctgcaaatgaacaacctgaaactggaggacacggccgtctatatctgtaatgcagaccacttcctcgaccagtttgattcttggggccaggggacccaggtcaccgtctcctca

SEQ ID NO: 23 - DNA, 378 bp, Lama glama
caggtgcagctgcaggagtctggaggaggattggtgcaggctgggggctcgctgagactctcctgtgtaacctctggatgtaccttcactacgtatgccatgggctggttccgccaggctccagggaaggagcgtgaatttgtagcagcagttacctggagtggtggtagcacatattatgcagattccgtgaagggccgattcaccatctccagagacaacggcaagaatacggtgtatttgcagatgaacagcctgaaacatggggacacggccgtttattactgtgcagcacgatatcccggaaagtggggtgaactgcctacggatactcggtttgacttctggggccaggggacccaggtcaccgtctcctca

SEQ ID NO: 24 - DNA, 366 bp, Lama glama
caggtgcagctgcaggagtctggaggaggattggtgccgactgggggctctctgagactctcctgtgcagcctctggacgcgacttcgagtcctatgccatgggctggttccgccaggctccagggaaggagcgtgagtttgtagcagctagtaggtggattggtggtcccacatactatgcaaactccgtgaaggcccgattcaccttctccggagacaacgccaagcgcacggtatatctgcaaatgaacagcctgaaacctgaggacacggccgtttattactgtgcagcggggggacgaatatctaatgagggcttcaagcctgactactggggccaggggacccaggtcaccgtctcctca

SEQ ID NO: 25 - DNA, 372 bp, Lama glama
caggtgcagctgcaggagtctgggggaggattggtgcaggctgggggctctctgagactctcctgtgcagcctctggagtcaccttcagtagatatgccatggcctggttccgccaggctccagggaaggagcgtgaatttgtcgcaggtaatagttggagcggtcgtaacgtaaactatgcagactccgtgaagggccgattcaccatcaccagagacaacgccaagaacacggtgtatctgcaaatgaatagcctgaaacctgaggacacggccgtttattactgtgcagcgagccaattcgggagtggtgccgactactcggaacaatatgactactggggccaggggacccaggtcaccgtctcctca

SEQ ID NO: 26 - DNA, 369 bp, Lama glama
caggtgcagctgcaggagtctgggggaggattggtgcaggctgggggctctctgaggctctcctgtgcagcctctggacgcaccttgtacgccatgggctggttccgtcaggctccagggaaggagcgtgagtttgtagcagctacaagctggaatggtggtaacacataccttccagactccgtgaagggccgattcaccatctccagagacaacgccaagaacacggtatatctgcaaatgaacagcctgaaacctgaggacacggccgtttatgtttgtgcagcttcgttccgggactcgcgtggtagtagtaattacgcccattatgactactggggccaggggacccaggtcaccgtctcctca

SEQ ID NO: 27 - DNA, 387 bp, Lama glama
caggtgcagctgcaggagtctggaggaggattggtacagactgggggctctctgagactctcctgtgcagcctctggacgcaccttcagtacctataatatgggctggttccgccaggctccagggaaggagcgtgagtttgtagcactgattaactggagtggtggtgacacatactatacaaactccgtgaagggccgattcaccatctccagagacaacgccaagaacacgttgtatctgcaaatgaacaacctgaaacctgaggacacggccgtttattcttgtgcggcgcgatatgcggccattggtagtccctactggaagcttacgactccgagtgagtacgaatactggggccaggggacccaggtcaccgtctcctca

SEQ ID NO: 28 - DNA, 380 bp, Lama glama
caggtgcagctgcaggagtctggggaggattggtgcaggctggggcctctctcagactctcctgtgcagcctctggacgcaccttcagtggctattacatgggctggttccgccaggctccagggaaggagcgtgaatttgtagcatttattaggtggagtggtggtgagacatactatacagactccgtgaagggccgattcaccatctccagagacaacaccaagaacacgatgtatctgcaaatgaacagtttgaaacctgaggacacggccgtttattactgtgcagcagataaggggatgggttactatcgcgacgctgccctcgcatcctggtatgactactggggccaggggacccaggtcaccgtctcctca

SEQ ID NO: 29 - DNA, 375 bp, Lama glama
caggtgcagctgcaggagtctggaggagggttggtggaggctgggggctctctgagactctcctgtgcagcctctggacgcaccagcgatatctatgccatggcctggttccgccaggctccagggcaggaccgtgagtttgtagcagctattacatggaatgatggtatcacatactattcatcctccgtgaagggccgattcaccatctccagagattacgccaagaacacggtgtatctacaaatgaacagcctgagtcctgaggacacggccgtttattacggtgcagtcacacgccgtagatacgttacactccgcgtcgctgatgactatgacaaatggggccaggggacccaggtcaccgtctcctca

SEQ ID NO: 30 - DNA, 372 bp, Lama glama
caggtgcagctgcaggagtctgggggagggttggtgcaggctgggggctctctgagactctcctgtgcagcctctggactgacctttggatacactgccacggcctggttccgccaggctccagggaaggagcgtgagtttgttgcgcgaattttcaaaagaggtggctacacatactattcagactccgtgaagggccgattcaccgtctccagagacagcgccaaggacacggtgtatctgcaaatgaacagcctgaaatctgaggacacggccgtttattactgtgcagtcgctcgggaatggagcgatctggactttcgagacggctatgactactggggtcaggggacccaggtcaccgtctcctca

SEQ ID NO: 31 - DNA, 378 bp, Lama glama
caggtgcagctgcaggagtctgggggaggattggtgcaggctgggggctcgctgagactctcctgtgcagcctctggacgcaccttcactacctatgtcatgggctggttccgccaggctccagggaaggagcgtgagtttgtagcagctgtttcctggaatggtggtagcacatactatgcagattccgtgaagggccgattcaccatctccagagacaacggcgagaacacggtgtatttgcagatgaacaccctgaaacatggggacacggccgtttattactgtgcagcacgatatcccggaaagtggggtgaactgcctacggatactcggtttgacttttggggccaggggacccaggtcaccgtctcctca

SEQ ID NO: 32 - DNA, 381 bp, Lama glama
caggtgcagctgcaggagtctggaggaggattggtgcaggctgggggctctctgagactctcctgtgtagcctctggacgcgccttcagtagcagttctatgggctggttccgccaggctcccgggaaggagcgtgagtttgtagcagctattaggtggagtgatgataatacagtctatgtagactccgtgaagggccgattcgccatctccagagacaacgccaagaacacggtgtatctgcaaatgaacagcctgaaacccgaggacacggccgtttattactgcgcagccacgggattggggtaccggtacgccgtcctggagtcgcgatctgagtatcgctactggggccaggggacccaggtcaccgtctcctca

SEQ ID NO: 33 - DNA, 384 bp, Lama glama
caggtgcagctgcaggagtctgggggaggcttggtgcaacctggggggtctctgagactctcctgtgcagcctcggcaagcatcgtcagtttcaatgccattggctggcaccgccaggctccagggaaggagcgcgagttggtcgcaagtatctccagtggtggtgtcgcaaactatgtagactccgtgaagggccgattcaccatctccagagacaacgccaagaacacggtgtatctgcaaatgaacagcctgaaacctgaggacacggccgtttatcactgtgcagcaaagaccaggggggtgtacggcactaactggctctccccagtcgactatgaatatggctcctggggccaggggacccaggtcaccgtctcctca

SEQ ID NO: 34 - DNA, 351 bp, Lama glama
caggtgcagctgcaggagtctgggggaggcttggtgcagcctggggggtctctgagactctcctgtgcagcctctggaagtatctctaatatgagagccatgaactggtaccgccagcagccagggaaggagcgcgagttggtcgcgcaaatatttctcactggtacaatttacattcgggactccgtgaagggccggtttaccatctccaaggacaacgccaagaacatggtatatctgcaaatgaacaacctgaaactggaggacacggccgtctatatctgcagtgcagaccacttcctcgaccagtttgattcttggggccaggggacccaggtcaccgtctcctca

SEQ ID NO: 35 - DNA, 372 bp, Lama glama
caggtgcagctgcaggagtctggaggaggattggtgcaggctgggggctctctgagactctcctgtgcagcctctggacgcaccttcagtagatccgccgtggcctggttccgccaggctccagggaaggagcgtgaatttgtcgcaggtattagctggagtggtcgtaacgtaaactatgcaaactccgtgaagggccgattcaccatctccagagacaataccaagaacacggtgtatctgcaaatgaacagcctgaagcctgaggacacggccgtttattactgtgcaaccagccaattcgggagtggtaccagcgactcgaaacagtatgactactggggccaggggacccaggtcaccgtctcctca

SEQ ID NO: 36 - DNA, 351 bp, Lama glama
caggtgcagctgcaggagtctgggggaggcttggtgcagcctggggggtctctgagactctcctgtgcagcctctggaagtatctccgatatgagagccatggactggtaccgccagcctccagggaaggagcgcgagttggtcgcgcaaatatttctcactggtactacttactatttggactccgtgaagggccgatttaccatctccaaggacaacgccaagaacacggtatatctgcaaatgaacaacctgaaagtggaggacacggccgtctatatctgtaatgcagaccacttcctcgaccagtttgattcttggggccaggggacccaggtcaccgtctcctca

SEQ ID NO: 37 - DNA, 348 bp, Lama glama
caggtgcagctgcaggagtctgggggaggcttggtgcagcctggggggtctctgagactctcctgtgcagcctctggaagcatcttccgagtcatgggctggtaccgccaggctccaggaaaggagcacgacttggtcgcaactattacaaggtctggtgacacaaactacgcagactccgtgaagggccgattcaccatctccagagacaacaccaagaatacggtgtatttacaaatgaacaacctgaaacctgaggacacggacgtctattactgctatgcacggaattttgtaggacataatccggacgtctggggccaggggacccaggtcaccgtctcctca

SEQ ID NO: 38 - DNA, 375 bp, Lama glama
caggtgcagctgcaggagtctggaggaggattggtgcaggctgggggctctctgagactcgactgtgcagcctctggattgaccttcagcaagtataccatggggtggttccgccaggctccagggaaggagcgggaatttgttgcagttatttcggggaatgatgatatcacatactatacagactccgtgaagggccgattcaccatctccagagacaatggcaagaacacggtgtatttgcaaatgaacagcctgaaacctgaggacacggccgtatattactgtgcagcagatcggggtgagagttactactacagccgtccgtctgagtatacctattggggccaggggacccaggtcaccgtctcctca

SEQ ID NO: 39 - DNA, 348 bp, Lama glama
caggtgcagctgcaggagtctggaggaggcctggtgcagcctggggggtctctgagactctcctgtgcagcctctggaagcatcttccgagtcatgggctggtaccgccaggctccaggaaaggagcacgagttggtcgcaactattacaaagtctggtgacacaaactacgcagactccgtgaagggccgattcaccatctccagagacaacgccaaggatacggtgtatttgcaaatgaacaacctgaaacctgaggacacggacgtctattactgctatgcacggaattttgtaggccgtaatccggacgtctggggccaggggacccaggtcaccgtctcctca

SEQ ID NO: 40 - DNA, 375 bp, Lama glama
caggtgcagctgcaggagtctggaggaggattggtgcaggctgggggctctctgagactctcctgtgcagcctctggacgcaccttcagtagcaccgccatgggctggttccgccaggctccagggaaggagcgtgaatttgtagcagttattagctggagtggtgctaacacacactattcagactccgtgaagggccgattcagcatctccagagacaacgccaagaacacggtgtatctgcaaatgaacagcctgaaacctgaggacacggccgtttattactgcgcagcagaccccgggaactttggggtacccgtcgagcattttttgtatgacttttggggccaggggacccaggtcaccgtctcctca

SEQ ID NO: 41 - DNA, 344 bp, Lama glama
caggtgcagctgcaggagtctgggggaggagtggtgcaggctggggactctctgagactctcctgtgcagtttctggacgcaccggcagaatggcctggttccgccaggctccagggaaggagcgtgagtttgtagcaggtattatgcggactggaagcttcacaaattatataggttccgtgaagggccgattcaccatctccatggacaacgccaagaacacagtgtatctgcaaatgaacagcctgaaagttgaggacacggccgtttattactgtgcagccacgggagacggtggttcgtatgactactggggccaggggacccaggtcaccgtctcctc

SEQ ID NO: 42 - DNA, 372 bp, Lama glama
caggtgcagctgcaggagtctgggggaggattggtgcaggctggggactctctgagactctcctgtgcagcctctggacgcacctttggaagccacgccacggcctggttccgccaggctccggggaaggagcgtgagtttgttgcacgaattttctggaggggtgattatacatactatgcagactccgtgaagggccgattcaccatctccagagacagcgccaagaacacggtgtatcttcaaatgaacagcctgaaatctgaggacacggccgtttattactgtgcagtcgctcggtcatggagcgatctggattttgaaaacgggtatgattactggggtcaggggacccaggtcaccgtctcctca

SEQ ID NO: 43 - DNA, 351 bp, Lama glama
caggtgcagctgcaggagtctggaggaggcttggtgcagcctggggggtctctgagactctcctgtgtagcctctggaagtatctccaatatgagagccatgaactggtaccgccagcctccagggaaggagcgcgagttggtcgcgcaaatatttctcactggtactatttactatcgggactccgtgaagggccgatttaccatctccaaggacaacgccaagaacacggtatatctgcaaatgaacaacctgaaactggaggacacggccgtctatagttgtagtgcagaccacttcctcgaccagtttgattcttggggccaggggacccaggtcaccgtctcctca

SEQ ID NO: 44 - DNA, 351 bp, Lama glama
caggtgcagctgcaggagtctgggggaggcttggtgcagcctggggggtctctgagactctcctgtgtagcctctggaagtatctccaatatgagagccatgaactggtaccgccagcctccagggaaggagcgcgagttggtcgcgcaaatatttctcactggtactatttactatcgggactccgtgaagggccgatttaccatctccaaggacaacgccaagaacacggtatatctgcaaatgaacaacctgaaactggaggacacggccgtctatagttgtagtgcagaccacttcctcgaccagtttgattcttggggccaggggacccaggtcaccgtctcctca

SEQ ID NO: 45 - DNA, 348 bp, Lama glama
caggtgcagctgcaggagtctggaggaggattggtgcagcctggggggtctctgagactctcctgtgcagcctctgcacacttcttcagtggcggcgccatgggctggtaccgccaggctccagggaaggagcgcgagttggtcgcaacaattactggtggcggggacaccaactatgctgagcacgtgaagggccgatgcaccatctccagcgacatatccgacaacacggtgtatctgcagatgatcagcctcaaacctgatgacacggccgtctattactgttatgcacgcgacctgcttcagcatcccttctggggccaggggacccaggtcaccgtctcctca

SEQ ID NO: 46 - DNA, 353 bp, Lama glama
caggtgcagctgcaggagtctgggggaggctgggtgcggcctggggggtctctgagactctcctgtgcagcctctggaagtatgttcagtctcagagtcatgggttggtaccgccaggctccagggaaggagcacgacttggtcgcaactattagtaggactggtatgacaaactatgcagactccgtgaagggccgattcaccatcccagagacaacgccaagaacacggtgtatctgcaaatgaacaacctgaaacctgaggacacggacgtctattactgctatgcacggaatttcgttggcgatgactttgatctctggggccaggggacccaggtcaccgtctcctca

SEQ ID NO: 47 - DNA, 375 bp, Lama glama
caggtgcagctgcaggagtctggaggaggattggtgcagactgggggctctctgagactctcctgtgcagcctctggacgcaccgccagtagttataccatgggctggttccgccaggctgcggggaaggagcgggactttgtagcagcaatacgcgagagtcttggatatacaaactatgcagactccgtgaagggccgattcaccatctccagagacaacgccaacaacacggtgtatctgcaaatgaacagcctgaaacctgaggacacggccgtttattactgtgcagcctatgaaggtattcgttactccagcacgagggctgatttttataactactggggtcaggggacccaggtcaccgtctcctca

SEQ ID NO: 48 - DNA, 375 bp, Lama glama
caggtgcagctgcaggagtctgggggaggattggtgcaggctgggggctctctgaggctctcctgcgcagcctctggatttaccttcggtagctcggccatgggctggttccgccagcctccaggaaaggagcgtgagtttgtagcaagtattaactggagtggtgttcatacacgccatgcagactccgtgaagggccgattcaccatctccagagacaacgccaagaacacggtgtatctgcaaatgaacagcctgaaacctgaggacacggccgtttattactgtgcaacagatccgggaaactttggactgcctaacagtgaattagagtttaactactggggccaggggacccaggtcaccgtctcctca

SEQ ID NO: 49 - DNA, 378 bp, Lama glama
caggtgcagctgcaggagtctgggggaggattggtgcaggctgggggctctctgagactctcctgtgcagcctctggacgcaccgtcagtttgcgtgtgatgggctggttccgccaggctccagggaaggagcgtgagtttgtagcgtttgtggactggaattcaggtaatgcatactattcagactccgtgcagggccgattcatcatgtccagagacaacgccgagaacacggcgtttctgcaaatgaacagcctgaaacctgaggacacggccgtttattactgcgcagcagatcctttttttcgagtcatcgcttatagcgaccctaaacgctatagctactggggccaggggacccaggtcaccgtctcctca

SEQ ID NO: 50 - DNA, 357 bp, Lama glama
caggtgcagctgcaggagtctgggggaggattggtgcgggctgggggctctctgagactctcctgtgcagcctctggacgcaccttgggtatttatcgtatgagctggttccgccaggctccagggaaggaacgtgaatttgtagcagctgttatgtggagtggtctacacccagtctatgcagactctgtgaaaggccgattcaccatctccggagacaacgccaagatgacgctatatctacaaatgaacaacctgacttctgaggacacggccacttattactgtgcagcccgacagccgaccactgcgacgtatgactactggggccaggggacccaggtcaccgtctcctca

SEQ ID NO: 51 - DNA, 354 bp, Lama glama
caggtgcagctgcaggagtctggaggaggcttggtgcagcctggggggtctctgagactctcctgtgcagcctctggaagcatcttcagcctcagagtcatgggctggtaccgccaaactccaggaaaggagcacgacttggtcgcaattattagtaggtctggtaacgcaaattacgcagactccgtgaagggccgattcaccatctccagagacaacgccaaaaatacggtgtatctgcaaatgaacaacctgaaacctgaagacacggacgtctattactgctatgcacggaattttataggcgataatccggacgtctggggccaggggacccaggtcaccgtctcctca

SEQ ID NO: 52 - DNA, 375 bp, Lama glama
caggtgcagctgcaggagtctgggggaggattggtgcaggctgggggctctctgagactctcctgtgcagcctctggacgcaccttcagtagctatgccatgggctggttccgccaggctccagggaaggagcgtgagtttgtagcagtgattaactggagtggtgctaacacacgctatacagactccgtgaagggccgattcagcatctccagagacaacgccaagaacacggtgtatctgcaaatgaacagcctgaaacctgaggacacggccgtttattactgtgcagcagaccccgggaactttggggtacccatcgagcattttatgtatgactactggggccaggggacccaggtcaccgtctcctca

SEQ ID NO: 53 - DNA, 348 bp, Lama glama
caggtgcagctgcaggagtctggaggaggcttggtgcagcctggggggtctctgagactctcctgtacagcctctggaagcatcttccgagtcatgggctggtaccgcctggctccaggaaaggattacgacttggtcgcaactattacagggtctggtgacacaaactacgcagagtccgtgaagggccgattcaccatctccagagacaacgccaagaatacggtgtatttgcaaatgaacaacctgagacctgaggacacggacgtctattactgctatgcacggaattttgtaggacgtaatccggacgtctggggccaggggacccaggtcaccgtctcctca

SEQ ID NO: 54 - protein, 125 aa, Lama glama
QVQLQESGGGLVQAGGSLHLSCTASGSENMLRWYAVGWYRQAPGKQRELVATITRGGSTNVVDSVKGRFTISRDNAKNMVALQMNSLKPEDTAVYSCAAAYAPYTDYHPVEYVYWGQGTQVTVSS

SEQ ID NO: 55 - protein, 126 aa, Lama glama
QVQLQESGGGLVQAGDSLRLSCAASGRTFDTHDIGWFRQAPGKEREFVAAMAWSGGSTYYADSVKGRFTISRDSAKNTVNLQMNSLKPEDTAVYYCASEAFQSSRLYSDYRRYHYWGQGTQVTVSS

SEQ ID NO: 56 - protein, 118 aa, Lama glama
QVQLQESGGGLVQPGGSLRLSCAASGSGFSLRVMGWYRQAPGKEHRLVATISRTGITNYADSVKGRFTISRDNAKDTVYLQMNNLKPEDTDVYYCYARNFVGDDFNLWGQGTQVTVSS

SEQ ID NO: 57 - protein, 117 aa, Lama glama
QVQLQESGGGLVQPGGSLRLSCAASGSISDMRAMNWYRQPPGKERELVAQIFLTGTIYYADSVKGRFTISKDNAKNTVYLQMNNLKLEDTAVYSCSADHFLDQFDSWGQGTQVTVSS

SEQ ID NO: 58 - protein, 123 aa, Lama glama
QVQLQESGGGLVQAGGSLRLSCAASGRTLYAMGWFRQAPGKEREFVAAKSWNGGNTYYADSVKGRFTISKDNAKNTVYLQMNSLKPEDTAVYNCAASSWDSRGSSNYVHYDYWGQGTQVTVSS

SEQ ID NO: 59 - protein, 118 aa, Lama glama
QVQLQESGGGLVQPGGSLRLSCAASTRIIFGVNAMGWYRQAPGKERELVARIEDGGTTNYADSVKGRFTISRDNAKNTVYLQMNSLKPEDTGIYYCNYHNSYYGSDYWGKGTQVTVSS

SEQ ID NO: 60 - protein, 124 aa, Lama glama
QVQLQESGGGLVQAGGSLRLSCAASGRTFSRSAVAWFRQAPGKEREFVAGISWSGRNVNYANSVKGRFTISRDSTKNTVYLQMNSLKPEDTAVYYCATSQFGSGTSDSEQYDYWGQGTQVTVSS

SEQ ID NO: 61 - protein, 121 aa, Lama glama
QVQLQESGGGSVQPGGSLRLSCAVSRSRFSLVTMGWYRQAPGKERELVAAIAIGGSTKYADSVKGRFTISRDNAKSTVYLQMNSLQPEDTAVYYCNEARVYDSNWYPRDYWGQGTQVTVSS

SEQ ID NO: 62 - protein, 125 aa, Lama glama
QVQLQESGGGLVQAGGSLRLSCAASGRTSDIYAMAWFRQTPGKDREFVAAITWNDGLTYYAGSVRGRFTISRDYAKNTVYLQMNNLKPEDTAVYYGAVTRRRFVTLRVPDDYDKWGQGTQVTVSS

SEQ ID NO: 63 - protein, 125 aa, Lama glama
QVQLQESGGGLVQAGDSLRLSCAASGFPFSSSAMGWFRQPPGKEREFVTSINWSGVHTRHADSVKGRFTISRDNAKNTVYLQMNSLKPEDTAVYYCATDPGNFGLPNSELEFNYWGQGTQVTVSS

SEQ ID NO: 64 - protein, 123 aa, Lama glama
  misc_feature (27)..(27): Xaa can be any naturally occurring amino acid
QVQLQESGGGLVQPGRSLRLSCAASGXIFSSYTIMGWFRQAPGKEREFVASISWSGGNIYYADSVKGRFTVSRDDAKNTVYLDMNNPKPEDTAVYYCASDLRMYEGDWYGGYWGQGTQVTVSS

SEQ ID NO: 65 - protein, 124 aa, Lama glama
QVQLQESGGGLVQAGGSLRLSCAASERTSGMHATAWFRQAPGKEREFVARIFHSGPYTYYADSVKGRFTIFRDSAKNTVDLQMNSLKSEDTAVYYCAVARSWSDLDFVDGYDYWGQGTQVTVSS

SEQ ID NO: 66 - protein, 117 aa, Lama glama
QVQLQESGGGLVQPGGSLRLSCAASTSVFGVNDMGWYRQAPEKERELVARISDGGTTNYADSVKGRFTISRDNAKNTVYLQMNNLKAEDTAIYYCNYHNYYYGLDFWGKGTQVTVSS

SEQ ID NO: 67 - protein, 125 aa, Lama glama
QVQLQESGGGLVQAGGSLRLSCAASGRTFSSTAMAWFRQAPGQEREFVATISWSGDNTKYADSVKGRFTISRDNAKNTVYLQMNSLKPEDTAVYYCAADPGNFGVPIEYFMFDYWGQGTQVTVSS

SEQ ID NO: 68 - protein, 121 aa, Lama glama
QVQLQESGGGLVQPGGSLRLSCVASGSIGSINHMAWFRKPPGKERELVASITLAPDEIAYYADSVKGRFAISKGNAQPTLYLQMNSLKPEDTAVYYCHFRQIRANAHDVYWGQGTQVTVSS

SEQ ID NO: 69 - protein, 126 aa, Lama glama
QVQLQESGGGLVQAGGSLRLSCAASGRTVSYRVMGWFRQAPGKEREFVAFVDWSSGTAYYAESVQGRFIMSRDTADNTGFLQMNNLKPEDTAVYYCAADPFFRVIAYSDPKRYSYWGQGTQVTVSS

SEQ ID NO: 70 - protein, 123 aa, Lama glama
QVQLQESGGGLVQAGGSLRLSCVASGRTLYAMGWFRQAPGKEREFVAATSWNGGNTNYGESVKGRFTISKDNAKNTVYLQMNSLKPEDTAVYNCAASSRDSRGTSTYASYDYWGQGTQVTVSS

SEQ ID NO: 71 - protein, 127 aa, Lama glama
QVQLQESGGGLVQAGDSLRLSCAASGRTFSSLSAGWFRQAPGKEREFVAAIRWSDYNTYYVDSVKGRFTISRDNAKNTVYLQMDSLKPEDTAVYYCAATALGYRYASLESRNEYRYWGQGTQVTVSS

SEQ ID NO: 72 - protein, 125 aa, Lama glama
QVQLQESGGGLVQAGGSLRLSCAASGRTSDIYAMAWFRQAPGKDREFVAAITWNNGEVYYSNSVKGRFTISRDYAKNTVYLQMNSLEPEDTAVYYGAATRRRYHTLRVSGDYDNWGQGTQVTVSS

SEQ ID NO: 73 - protein, 121 aa, Lama glama
QVQLQESGGGLVQPGGSLRLSCVASRSIGSINHMAWYRKAPGKERELVASITLSPSETVYYTEVVKGRFAISKGNAQNTLYLQMNSLKPEDTAVYYCHFRQIRAIADDVYWGQGTQVTVSS

SEQ ID NO: 74 - protein, 122 aa, Lama glama
QVQLQESGGGLVQPGGSLSLSCTTSGEYHTINYMGWYRQAPGKERELVARISFGGRIEYGDFVKGRFTLSRNNAKNTVNLQMNSLKPEDTAVYYCNGEITTLISVLSTDQYWGQGTQVTVSS

SEQ ID NO: 75 - protein, 117 aa, Lama glama
QVQLQESGGGLVQPGGSLRLSCAASGSISDMRAMNWYRQPPGKESELVAQIFLTGTIYYRDSVKGRFTISKDNAKNTVYLQMNNLKLEDTAVYICNADHFLDQFDSWGQGTQVTVSS

SEQ ID NO: 76 - protein, 126 aa, Lama glama
QVQLQESGGGLVQAGGSLRLSCVTSGCTFTTYAMGWFRQAPGKEREFVAAVTWSGGSTYYADSVKGRFTISRDNGKNTVYLQMNSLKHGDTAVYYCAARYPGKWGELPTDTRFDFWGQGTQVTVSS

SEQ ID NO: 77 - protein, 122 aa, Lama glama
QVQLQESGGGLVPTGGSLRLSCAASGRDFESYAMGWFRQAPGKEREFVAASRWIGGPTYYANSVKARFTFSGDNAKRTVYLQMNSLKPEDTAVYYCAAGGRISNEGFKPDYWGQGTQVTVSS

SEQ ID NO: 78 - protein, 124 aa, Lama glama
QVQLQESGGGLVQAGGSLRLSCAASGVTFSRYAMAWFRQAPGKEREFVAGNSWSGRNVNYADSVKGRFTITRDNAKNTVYLQMNSLKPEDTAVYYCAASQFGSGADYSEQYDYWGQGTQVTVSS

SEQ ID NO: 79 - protein, 123 aa, Lama glama
QVQLQESGGGLVQAGGSLRLSCAASGRTLYAMGWFRQAPGKEREFVAATSWNGGNTYLPDSVKGRFTISRDNAKNTVYLQMNSLKPEDTAVYVCAASFRDSRGSSNYAHYDYWGQGTQVTVSS

SEQ ID NO: 80 - protein, 129 aa, Lama glama
QVQLQESGGGLVQTGGSLRLSCAASGRTFSTYNMGWFRQAPGKEREFVALINWSGGDTYYTNSVKGRFTISRDNAKNTLYLQMNNLKPEDTAVYSCAARYAAIGSPYWKLTTPSEYEYWGQGTQVTVSS

SEQ ID NO: 81 - protein, 126 aa, Lama glama
QVQLQVWGGLVQAGASLRLSCAASGRTFSGYYMGWFRQAPGKEREFVAFIRWSGGETYYTDSVKGRFTISRDNTKNTMYLQMNSLKPEDTAVYYCAADKGMGYYRDAALASWYDYWGQGTQVTVSS

SEQ ID NO: 82 - protein, 125 aa, Lama glama
QVQLQESGGGLVEAGGSLRLSCAASGRTSDIYAMAWFRQAPGQDREFVAAITWNDGITYYSSSVKGRFTISRDYAKNTVYLQMNSLSPEDTAVYYGAVTRRRYVTLRVADDYDKWGQGTQVTVSS

SEQ ID NO: 83 - protein, 124 aa, Lama glama
QVQLQESGGGLVQAGGSLRLSCAASGLTFGYTATAWFRQAPGKEREFVARIFKRGGYTYYSDSVKGRFTVSRDSAKDTVYLQMNSLKSEDTAVYYCAVAREWSDLDFRDGYDYWGQGTQVTVSS

SEQ ID NO: 84 - protein, 126 aa, Lama glama
QVQLQESGGGLVQAGGSLRLSCAASGRTFTTYVMGWFRQAPGKEREFVAAVSWNGGSTYYADSVKGRFTISRDNGENTVYLQMNTLKHGDTAVYYCAARYPGKWGELPTDTRFDFWGQGTQVTVSS

SEQ ID NO: 85 - protein, 127 aa, Lama glama
QVQLQESGGGLVQAGGSLRLSCVASGRAFSSSSMGWFRQAPGKEREFVAAIRWSDDNTVYVDSVKGRFAISRDNAKNTVYLQMNSLKPEDTAVYYCAATGLGYRYAVLESRSEYRYWGQGTQVTVSS

SEQ ID NO: 86 - protein, 128 aa, Lama glama
QVQLQESGGGLVQPGGSLRLSCAASASIVSFNAIGWHRQAPGKERELVASISSGGVANYVDSVKGRFTISRDNAKNTVYLQMNSLKPEDTAVYHCAAKTRGVYGTNWLSPVDYEYGSWGQGTQVTVSS

SEQ ID NO: 87 - protein, 117 aa, Lama glama
QVQLQESGGGLVQPGGSLRLSCAASGSISNMRAMNWYRQQPGKERELVAQIFLTGTIYIRDSVKGRFTISKDNAKNMVYLQMNNLKLEDTAVYICSADHFLDQFDSWGQGTQVTVSS

SEQ ID NO: 88 - protein, 124 aa, Lama glama
QVQLQESGGGLVQAGGSLRLSCAASGRTFSRSAVAWFRQAPGKEREFVAGISWSGRNVNYANSVKGRFTISRDNTKNTVYLQMNSLKPEDTAVYYCATSQFGSGTSDSKQYDYWGQGTQVTVSS

SEQ ID NO: 89 - protein, 117 aa, Lama glama
QVQLQESGGGLVQPGGSLRLSCAASGSISDMRAMDWYRQPPGKERELVAQIFLTGTTYYLDSVKGRFTISKDNAKNTVYLQMNNLKVEDTAVYICNADHFLDQFDSWGQGTQVTVSS

SEQ ID NO: 90 - protein, 116 aa, Lama glama
QVQLQESGGGLVQPGGSLRLSCAASGSIFRVMGWYRQAPGKEHDLVATITRSGDTNYADSVKGRFTISRDNTKNTVYLQMNNLKPEDTDVYYCYARNFVGHNPDVWGQGTQVTVSS

SEQ ID NO: 91 - protein, 125 aa, Lama glama
QVQLQESGGGLVQAGGSLRLDCAASGLTFSKYTMGWFRQAPGKEREFVAVISGNDDITYYTDSVKGRFTISRDNGKNTVYLQMNSLKPEDTAVYYCAADRGESYYYSRPSEYTYWGQGTQVTVSS

SEQ ID NO: 92 - protein, 116 aa, Lama glama
QVQLQESGGGLVQPGGSLRLSCAASGSIFRVMGWYRQAPGKEHELVATITKSGDTNYADSVKGRFTISRDNAKDTVYLQMNNLKPEDTDVYYCYARNFVGRNPDVWGQGTQVTVSS

SEQ ID NO: 93 - protein, 125 aa, Lama glama
QVQLQESGGGLVQAGGSLRLSCAASGRTFSSTAMGWFRQAPGKEREFVAVISWSGANTHYSDSVKGRFSISRDNAKNTVYLQMNSLKPEDTAVYYCAADPGNFGVPVEHFLYDFWGQGTQVTVSS

SEQ ID NO: 94 - protein, 115 aa, Lama glama
QVQLQESGGGVVQAGDSLRLSCAVSGRTGRMAWFRQAPGKEREFVAGIMRTGSFTNYIGSVKGRFTISMDNAKNTVYLQMNSLKVEDTAVYYCAATGDGGSYDYWGQGTQVTVSS

SEQ ID NO: 95 - protein, 124 aa, Lama glama
QVQLQESGGGLVQAGDSLRLSCAASGRTFGSHATAWFRQAPGKEREFVARIFWRGDYTYYADSVKGRFTISRDSAKNTVYLQMNSLKSEDTAVYYCAVARSWSDLDFENGYDYWGQGTQVTVSS

SEQ ID NO: 96 - protein, 117 aa, Lama glama
QVQLQESGGGLVQPGGSLRLSCVASGSISNMRAMNWYRQPPGKERELVAQIFLTGTIYYRDSVKGRFTISKDNAKNTVYLQMNNLKLEDTAVYSCSADHFLDQFDSWGQGTQVTVSS

SEQ ID NO: 97 - protein, 117 aa, Lama glama; identical to SEQ ID NO: 96

SEQ ID NO: 98 - protein, 116 aa, Lama glama
QVQLQESGGGLVQPGGSLRLSCAASAHFFSGGAMGWYRQAPGKERELVATITGGGDTNYAEHVKGRCTISSDISDNTVYLQMISLKPDDTAVYYCYARDLLQHPFWGQGTQVTVSS

SEQ ID NO: 99 - protein, 118 aa, Lama glama
QVQLQESGGGWVRPGGSLRLSCAASGSMFSLRVMGWYRQAPGKEHDLVATISRTGMTNYADSVKGRFTISRDNAKNTVYLQMNNLKPEDTDVYYCYARNFVGDDFDLWGQGTQVTVSS

SEQ ID NO: 100 - protein, 125 aa, Lama glama
QVQLQESGGGLVQTGGSLRLSCAASGRTASSYTMGWFRQAAGKERDFVAAIRESLGYTNYADSVKGRFTISRDNANNTVYLQMNSLKPEDTAVYYCAAYEGIRYSSTRADFYNYWGQGTQVTVSS

SEQ ID NO: 101 - protein, 125 aa, Lama glama
QVQLQESGGGLVQAGGSLRLSCAASGFTFGSSAMGWFRQPPGKEREFVASINWSGVHTRHADSVKGRFTISRDNAKNTVYLQMNSLKPEDTAVYYCATDPGNFGLPNSELEFNYWGQGTQVTVSS

SEQ ID NO: 102 - protein, 126 aa, Lama glama
QVQLQESGGGLVQAGGSLRLSCAASGRTVSLRVMGWFRQAPGKEREFVAFVDWNSGNAYYSDSVQGRFIMSRDNAENTAFLQMNSLKPEDTAVYYCAADPFFRVIAYSDPKRYSYWGQGTQVTVSS

SEQ ID NO: 103 - protein, 119 aa, Lama glama
QVQLQESGGGLVRAGGSLRLSCAASGRTLGIYRMSWFRQAPGKEREFVAAVMWSGLHPVYADSVKGRFTISGDNAKMTLYLQMNNLTSEDTATYYCAARQPTTATYDYWGQGTQVTVSS

SEQ ID NO: 104 - protein, 118 aa, Lama glama
QVQLQESGGGLVQPGGSLRLSCAASGSIFSLRVMGWYRQTPGKEHDLVAIISRSGNANYADSVKGRFTISRDNAKNTVYLQMNNLKPEDTDVYYCYARNFIGDNPDVWGQGTQVTVSS

SEQ ID NO: 105 - protein, 125 aa, Lama glama
QVQLQESGGGLVQAGGSLRLSCAASGRTFSSYAMGWFRQAPGKEREFVAVINWSGANTRYTDSVKGRFSISRDNAKNTVYLQMNSLKPEDTAVYYCAADPGNFGVPIEHFMYDYWGQGTQVTVSS

SEQ ID NO: 106 - protein, 116 aa, Lama glama
QVQLQESGGGLVQPGGSLRLSCTASGSIFRVMGWYRLAPGKDYDLVATITGSGDTNYAESVKGRFTISRDNAKNTVYLQMNNLRPEDTDVYYCYARNFVGRNPDVWGQGTQVTVSS

SEQ ID NO: 107 - DNA, 23 bp, Artificial Sequence
  CALL001 Primer
gtcctggctgctcttctacaagg

SEQ ID NO: 108 - DNA, 23 bp, Artificial Sequence
  CALL002 Primer
ggtacgtgctgttgaactgttcc

SEQ ID NO: 109 - DNA, 38 bp, Artificial Sequence
  VHH-Back-SAPI Primer
cttggctcttctgtgcagctgcaggagtctggrggagg

SEQ ID NO: 110 - DNA, 35 bp, Artificial Sequence
  VHH-Fw-SAPI Primer
tgatgctcttccgctgaggagacggtgacctgggt

(19) United States
(12) Patent Application Publication
(10) Pub. No.: US 2024/0254202 A1
(43) Pub. Date: Aug. 1, 2024

(54) LLAMA-DERIVED NANOBODIES BINDING THE SPIKE PROTEIN OF NOVEL CORONAVIRUS SARS-COV-2 WITH NEUTRALIZING ACTIVITY AND APPLICATION THEREOF

(71) Applicants: CONSEJO NACIONAL DE INVESTIGACIONES CIENTÍFICAS Y TÉCNICAS (CONICET), Ciudad Autónoma de Buenos Aires (AR); INSTITUTO NACIONAL DE TECNOLOGÍA AGROPECUARIA, Ciudad Autónoma de Buenos Aires (AR); VIRGINIA TECH INTELLECTUAL PROPERTIES, INC., Blacksburg, VA (US)

(72) Inventors: Gladys Viviana PARREÑO, Hurlingham (AR); Lorena Itati IBAÑEZ, Ciudad Autónoma de Buenos Aires (AR); Marina BOK, Ituzaingo (AR); Florencia PAVAN, Ciudad Autónoma de Buenos Aires (AR); Juan Pablo MALITO, Castelar, Buenos Aires (AR); Andrés WIGDOROVITZ, Florida (AR); Gisela MARCOPPIDO, El Palomar (AR); Diego FRANCO, Hurlingham (AR); Elsa BAUMEISTER, Ciudad Autónoma de Buenos Aires (AR); Juan Manuel SCHAMMAS, Ciudad Autónoma de Buenos Aires (AR); Lijuan YUAN, Blacksburg, VA (US); Jonathan AUGUSTE, Blacksburg, VA (US)

(21) Appl. No.: 18/269,156

(22) PCT Filed: Dec. 21, 2021
(86) PCT No.: PCT/US21/64662

§ 371 Date: Jun. 22, 2023

Related U.S. Application Data
(60) Provisional application No. 63/129,118, filed on Dec. 22, 2020.

(51) Int. Cl. C07K 16/10; A61K 39/00; A61P 31/12

(52) U.S. Cl. CPC C07K 16/1003; A61P 31/12; A61K 2039/507; C07K 2317/22; C07K 2317/34; C07K 2317/569; C07K 2317/76

(57) ABSTRACT

This disclosure is related to llama-derived nanobodies of which the corresponding sequences are provided, corresponding to the variable domain (VHH) of llama heavy chain antibodies recognizing epitopes of the Spike protein of SARS-COV-2, some of them, directed to the receptor binding domain, RBD. The nanobodies disclosed herein have important scientific significance and application prospects, they can be labeled or fused with fluorochromes and enzymes to be used as reagents for immunodetection of the virus. Most importantly, the disclosed nanobodies can be used in the development of a bio-drug for the prevention and clinical treatment of infection and diseases caused by SARS-COV-2.

### TECHNICAL FIELD OF THE INVENTION

The invention relates to the field of biological medicine, to nanobodies, obtained from an immune library derived from a llama immunized with the spike and RBD, directing at coronavirus SARS-CoV-2 and application thereof.

### DESCRIPTION OF THE PRIOR ART

In 2019-2020, a novel coronavirus infectious disease (COVID-19) caused by a coronavirus (Severe Acute Respiratory Syndrome Coronavirus 2, SARS-COV-2) affected most of the countries around the world becoming a pandemic.

Regarding morbidity and mortality, the infection spread very fast reaching fatality rate around 6%

SARS COV-2 is a member of the β genus Coronavirus (Betaconovir), carrying a positive-stranded RNA genome within a capsid of about 80-120 nm in diameter covered by a lipid bilayer envelop. The envelop contains 3 kinds of envelope glycoproteins, the Spike protein (S), the E protein, and the M protein. The virions are typically characterized by exhibiting a “crown” like morphology under electron microscopy, which is formed by numerous Spike proteins distributed on the viral envelope. The Spike protein is organized as trimers on the viral envelop, and each monomeric structure is composed of 1273 amino acids divided into two regions: the S1 subunit and one S2 subunit. It has been found that the Receptor Binding Domain (RBD) of the S1 protein on the Spike protein trimer exists in “closed” and “open” states. When in the “open” state, one RBD in the trimer is in an extended state. This fine change in conformation mediates recognition and binding of the Spike protein to the cell receptor to initiate the infection.

Recent reports indicated that, like other coronaviruses, the host receptor of SARS-COV-2 is the angiotensin converting enzyme 2 (ACE2). This receptor is expressed in a great variety of tissues and is especially abundant in lung and small intestine tissues. During the infection, the RBD located in the S1 subunit of the spike protein of SARS-COV-2 interacts with ACE2 receptor and further promotes the fusion of the virus with the host cell membrane, mediating the virus invasion of the host cell.

At present a great effort in being made to develop and test different type of vaccines to fight the pandemic. However, the poor (low magnitude and short duration) immune response to natural infection and the appearance in Asia and Europe of a second round of infections during the winter of 2020, awake a strong interrogation regarding the efficacy of the vaccines and pose the need to develop other type of preventive and well as curative treatments. The use of antibodies represents an excellent alternative and complement strategy to control de disease. In this regard, several approaches using monoclonal and polyclonal antibodies are explored by researchers all over the world.

Antibodies are a class of immunoglobulins that specifically bind to an antigen. Antibody drugs generally refer to the collective name of whole antibody molecules and antibody fragments with therapeutic functions obtained by advanced technology, and are one of the important means of targeted therapy.

Llama-derived nanobodies (NAbs), also recognized as the variable domain of the heavy chain antibodies (VHHs), comprise only the variable region of the antibody heavy chain present in the serum of camelids.

These nanobodies, also known as single domain antibodies, exhibit various advantages such as high expression yields, weak immunogenicity, easy production, and preparation. Single domain antibodies can also recognize the fine structure hidden on the surface of the antigen, can accurately aim and capture a target spot, and can be specifically combined with a target molecule.

Through years of research, single domain antibodies have shown excellent molecular characteristics and druggability. Single domain antibodies have not only the antigen binding ability of traditional antibodies, but also a plurality of unique properties compared thereto, such as good stability, capability of reaching special antigen epitopes, random combination of block modes, low production cost and the like.

The conventional method for obtaining single-domain antibodies consists of numerous steps of multiple immunizations of camelids, B lymphocyte isolation, VHH region amplification, phage library construction and screening. With the development of synthetic biology, it became possible to construct high-quality randomized high-capacity single-domain antibody libraries based on total synthetic humanization.

Chi, X. et al. (Humanized single domain antibodies neutralize SARS-COV-2 by targeting the spike receptor binding domain. Nat Commun 11, 4528 (2020). R: 29 Mar. 2020. https://doi.org/10.1038/s41467-020-18387-8) describe neutralizing antibodies providing efficient blockade for viral infection which are a promising category of biological therapies. The document describes the use of SARS-COV-2 spike receptor-binding domain (RBD) as a bait, to generate a panel of humanized single domain antibodies (sdAbs) from a synthetic library. It also teaches that fusion of the human IgG1 Fc to sdAbs improves their neutralization activity by up to ten times. The results support neutralizing sdAbs as a potential alternative for antiviral therapies.

Jianbo Dong et al. (2020) (Development of multi-specific humanized llama antibodies blocking SARS-COV-2/ACE2 interaction with high affinity and avidity, Emerging Microbes & Infections, 9:1, 1034-1036, DOI: 10.1080/22221751.2020.1768806) describe that blocking the interaction of the Spike protein with the ACE2 receptor with antibodies is a promising prospect for treatment against SARS-COV-2. The document describes the use of humanized llama antibody VHHs against SARS-COV-2 that would overcome the limitations associated with polyclonal and monoclonal combination therapies. From two llama VHH libraries, unique humanized VHHs that bind to S protein and block the S/ACE2 interaction were identified. The document also teaches the in-silico development of multi-specific antibodies with enhanced affinity and avidity, and improved S/ACE2 blocking using an approach that fuses VHHs to Fc domains. The authors specifically disclose a bi-specific antibody with potent S/ACE2 blocking.

Gai, Junwei et al. (2020). (A potent neutralizing nanobody against SARS-COV-2 with inhaled delivery potential. DOI: 10.1101/2020.08.09.242867) disclose nanobody (Nb) phage display libraries derived from four camels immunized with the SARS-COV-2 spike receptor-binding domain (RBD), from which 381 Nbs were identified to recognize SARS-COV-2-RBD. The authors teach that seven Nbs were shown to block interaction of human angiotensin converting enzyme 2 (ACE2) with SARS-COV-2-RBD-variants.

Huo, J. et al. (Neutralizing nanobodies bind SARS-COV-2 spike RBD and block interaction with ACE2. Nat Struct Mol Biol 27, 846-854 (2020). https://doi.org/10.1038/s41594-020-0469-6) disclose the use of a naive llama single-domain antibody library and PCR-based maturation to produce two closely related nanobodies that bind RBD and block its interaction with ACE2.

Leo Hanke et al. (An alpaca nanobody neutralizes SARS-COV-2 by blocking receptor interaction, bioRxiv 2020.06.02.130161; doi: https://doi.org/10.1101/2020.06.02.130161) disclose the isolation and characterization of an alpaca-derived single-domain antibody fragment that specifically targets the receptor binding domain (RBD) of the SARS-COV-2 spike, directly preventing ACE2 engagement.

Daniel Wrapp et al. (Structural Basis for Potent Neutralization of Betacoronaviruses by Single-Domain Camelid Antibodies, Cell, Volume 181, Issue 5, 2020, Pages 1004-1015.e15, ISSN 0092-8674, https://doi.org/10.1016/j.cell.2020.04.031) disclose the isolation of single-domain antibodies (VHHs) from a llama immunized with prefusion-stabilized coronavirus spikes. These VHHs neutralize MERS-COV or SARS-COV-1 S pseudotyped viruses, respectively. The document also teaches that a cross-reactive VHH neutralizes SARS-COV-2 S pseudotyped viruses as a bivalent human IgG Fc-fusion.

Guillermo Valenzuela Nieto et al. (Fast isolation of sub-nanomolar affinity alpaca nanobody against the Spike RBD of SARS-COV-2 by combining bacterial display and a simple single-step density gradient selection. bioRxiv 2020.06.09.137935; doi: https://doi.org/10.1101/2020.06.09.137935) disclose the development of a fast track for nanobody isolation against the receptor-binding-domain (RBD) SARS-COV-2 Spike protein following an optimized immunization, efficient construction of the VHH library for E. coli surface display, and single-step selection of high-affinity nanobodies using a simple density gradient centrifugation of the bacterial library. Following this procedure, the authors isolate and characterize an alpaca Nanobody against Spike RBD of SARS-COV-2 in the sub-nanomolar range.

Michael Schoof et al. (An ultra-high affinity synthetic nanobody blocks SARS-COV-2 infection by locking Spike into an inactive conformation. bioRxiv 2020.08.08.238469; doi: https://doi.org/10.1101/2020.08.08.238469) disclose single-domain antibodies obtained by screening a yeast surface-displayed library of synthetic nanobody sequences, which potently disrupt the interaction between the SARS-COV-2 Spike and ACE2.

Justin D. Walter et al. (Synthetic nanobodies targeting the SARS-COV-2 receptor-binding domain. bioRxiv 2020.04.16.045419; doi: https://doi.org/10.1101/2020.04.16.045419) disclose the generation of synthetic nanobodies, known as sybodies, against the receptor-binding domain (RBD) of SARS-COV-2. The sybodies were selected entirely in vitro from three large combinatorial libraries, using ribosome and phage display.

Wu Y et al. (Identification of Human Single-Domain Antibodies against SARS-COV-2. Cell Host Microbe. 2020 Jun 10;27(6):891-898.e5. doi: 10.1016/j.chom.2020.04.023. Epub 2020 May 14. PMID: 32413276; PMCID: PMC7224157) disclose the development of a phage-displayed single-domain antibody library by grafting naive complementarity-determining regions (CDRs) into framework regions of a human germline immunoglobulin heavy chain variable region (IGHV) allele. Panning this library against SARS-COV-2 RBD and S1 subunit identified fully human single-domain antibodies targeting five distinct epitopes on SARS-COV-2 RBD with subnanomolar to low nanomolar affinities.

Patent applications CN111647077A. CN111647076A and CN111303279A disclose single-domain antibodies for blocking the binding of the SARS-COV-2 Spike protein with the human cell ACE2 receptor.

Nevertheless, the further development of recombinant monoclonal antibodies, such as llama-derived nanobodies with neutralization activity continues to be an urgent need tool for preventing and treating COVID-19.

### SUMMARY OF THE INVENTION

It is an aspect of the present invention to provide a SARS-COV-2 Spike protein binding molecule comprising at least one immunoglobulin single variable domain.

Preferably, the SARS-COV-2 Spike protein binding molecule is a single domain antibody. More preferably, the single domain antibody has an amino acid sequence as set forth in any of SEQ ID NO: 54 to SEQ ID NO: 106. More preferably, the SARS-COV-2 Spike protein binding molecule is a single domain antibody having an amino acid sequence selected from the group consisting of SEQ ID NOs: 67, 68, 90, 95, 98, 100 and 105. Even more preferably, the SARS-COV-2 Spike protein binding molecule is a single domain antibody having an amino acid sequence selected from the group consisting of SEQ ID NOs: 67, 68, 90 and 95.

It is another aspect of this invention to provide a nucleic acid molecule encoding a SARS-COV-2 Spike protein binding molecule according to the invention.

In a particular embodiment, the nucleic acid molecule has a nucleotide sequence as set forth in any of SEQ ID NO: 1 to SEQ ID NO: 53. Preferably, the nucleic acid molecule has a nucleotide sequence selected from the group consisting of SEQ ID NOs: 14, 15, 37, 42, 45, 47, and 52. More preferably, the nucleic acid molecule has a nucleotide sequence selected from the group consisting of SEQ ID NOs: 14, 15, 37 and 42.

It is yet another aspect of the invention to provide a pharmaceutical composition comprising a SARS-COV-2 Spike protein binding molecule according to the invention.

It is yet another aspect of the invention to provide a pharmaceutical composition comprising at least two SARS-COV-2 Spike protein binding molecules according to the invention. In a preferred embodiment of this aspect of the invention, the pharmaceutical composition comprises a SARS-CoV-2 Spike protein binding molecule having the amino acid sequence set forth in SEQ ID NO: 67 and a SARS-COV-2 Spike protein binding molecule having the amino acid sequence set forth in SEQ ID NO: 68. In another preferred embodiment of this aspect of the invention, the pharmaceutical composition comprises a SARS-COV-2 Spike protein binding molecule having the amino acid sequence set forth in SEQ ID NO: 95 and a SARS-COV-2 Spike protein binding molecule having the amino acid sequence set forth in SEQ ID NO: 105.

It is yet another aspect of the invention to provide the use of a SARS-COV-2 Spike protein binding molecule according to the invention for the manufacture of a medicament for preventing and/or treating a disease caused by infection by novel coronavirus SARS-COV-2.

In a particular embodiment, preventing and/or treating a disease caused by infection by novel coronavirus SARS-COV-2 comprises inhibiting the infection of a subject by novel coronavirus SARS-COV-2.

It is yet another aspect of the invention to provide a method for preventing and/or treating a disease caused by infection by novel coronavirus SARS-COV-2, comprising administering a therapeutically effective amount of a SARS-COV-2 Spike protein binding molecule or a pharmaceutical composition according to the invention to a subject in need thereof.

In a particular embodiment, the disease caused by the infection by novel coronavirus SARS-COV-2 is selected from the group consisting of human novel coronavirus pneumonia, intestinal disease, intra vascular disseminated coagulation, and encephalitis.

It is yet another aspect of the invention to provide a use of a SARS-COV-2 Spike protein binding molecule according to the invention as a diagnosis reagent.

It is yet another aspect of the invention to provide a use of a SARS-COV-2 Spike protein binding molecule according to the invention as a capture reagent in the Spike purification process or as a concentrator of low amount of virus in a sample. In a particular embodiment, the sample is selected from the group consisting of food, water, feces, blood, serum and body secretions.

It is yet another aspect of the present invention to provide a method for preparing a SARS-COV-2 Spike protein binding molecule according to the invention, comprising screening a phage library.

### BRIEF DESCRIPTION OF THE DRAWINGS

FIG. 1. Schedule for llama immunization and sampling.

FIG. 2. Total IgG antibody response to Spike and RBD in llama's serum over time by ELISA. (a) Neutralizing antibody titer in serum of the immunized llama. The arrows point out immunization doses. (b) Viral neutralization assay using pseudotyped particles. Green fluorescence indicates the transduction of GFP and Spike protein in the cells infected with pseudotyped lentiviruses. (c) Evolution of the VN Ab titers over time.

FIG. 3. Construction and quality control of the VHH gene library for SARS COV.2 Spike. (a) Gels obtained after purification of RNA from lymphocytes of the llama immunized with the Spike proteins and RBD (A), DNA amplification by a first PCR where the upper band corresponds to the fragment of the variable regions plus the CH1 region of conventional antibodies and the band below the variable regions of the heavy chain antibodies (B), and second PCR where the VHHs regions are specifically amplified (C). (b) Gels obtained after colony-PCR amplification of individual clones of the llama library immunized with Spike and RBD proteins, it allows to determine the percentage of positive clones of the library, in this case, 100% (Panel A in the left). Determination of library diversity by PCR amplification followed by restriction with the frequent cutting enzyme Hinfl (Panel B, right). The obtained VHH gene library showed a size of 10⁹ clones and very high diversity. (c) Phagemid vector used to construct the VHH library. This phagemid vector is also the expression vector for transforming WK6 E. coli and producing VHH-6xhis tag.

FIG. 4. Specific Phage ELISA and Periplasmic extract ELISA assays to SARS-COV-2-SARS-CoV-2 RBD and S protein of clones selected with the full length pre-fused and locked spike protein. (a) RBD biopanning. (b) Spike biopanning.

FIG. 5. Nanoantibody expression and purification. (A) and (B) SDS PAGE, Coomassie Blue staining. FT: flow through. L1 and L2: washes, E1, E2, E3, E4 fractions eluted from Ni-NTA affinity chromatography. The red arrow indicates the presence of eluted nanoantibody. (C) Western blot of Spike protein run on an SDS-PAGE under non reducing condition, nanobodies and an anti-his antibody coupled to HRP. Nanobodies 10, 35, 39, 43, 104, 110, and 125 recognized linear epitopes. (D) and (E) Second step of molecular exclusion purification (chromatograms of two exemplary nanobodies, Akta-prime) and (F) quality control SDS-PAGE where the pre- and post-size exclusion chromatography (SEC) purity increment is clearly observed.

FIG. 6. (a) Dose response curves and the EC50 dose of the nanobodies directed to epitopes in RBD in an ELISA against Spike and RBD. (b) Dose response curve and the EC50 dose of the nanobodies directed to epitopes in S not in RBD.

FIG. 7. Neutralizing effect of a single domain antibody of the invention against pseudotyped lentiviruses expressing S and GFP. (a) Example clones with different neutralizing capacity as 29, 33, 43 or non-neutralizing, 32, 44, 73, 78, 85. (b) Dose response curves of neutralization for each clone with the increment of the Nb concentration.

FIG. 8. ELISA and VN results for nanobodies combinations. (a) ELISA-RBD results for the equimolar combination of Nb 39 and Nb 43. (b) ELISA-RBD results for the equimolar combination of Nb 110 and Nb 145. (c) VN results for 1:3 Nb 39:Nb 43 combinations. (d) ELISA-RBD results for the equimolar combination of Nbs 39, 43 and 110.

FIG. 9. Blocking assay between ACE2 and RBD. Competitive ELISA ACE2-RBD results for selected neutralizing nanobodies interfering with the binding of RBD to ACE2.

FIG. 10. Assay on the interference of the binding of biliverdin to Spike. (a) Results with varying nanobody concentration. (b) Results with varying biliverdin concentration.

FIG. 11. Mice experiment results. (a) Survival curve. 80% of the challenge animals that receive prophylactic treatment with the Nb 39 were protected from death. The groups receiving Nb 110, 104 and 43 showed protection rates between 60 and 40% in the doses administered. Animals receiving the non-neutralizing clones Nb 33 and 45 did not survive. (b) Weight gain. The animals receiving neutralizing nanobodies maintained or gained weight.

### DETAILED DESCRIPTION OF THE INVENTION

Pointing at the limitation that existing antiviral drugs and antibodies have no specificity to the novel coronavirus SARS-COV-2, and that they thus show limited or null preventive and therapeutic effect against SARS COV-2 infection and disease, the present invention provides SARS-COV-2 Spike protein binding molecules and applications thereof.

The inventors have found that the SARS-COV-2 Spike protein binding molecules of the present invention exhibit the following advantageous properties:
i) they block or exclude coronavirus SARS-Cov-2 infection and disease, with high-efficiency antiviral capacity for the novel coronavirus SARS-Cov-2; and
ii) they recognize with high affinity different epitopes within the Spike protein having application in diagnosis and other applications.

It is therefore an aspect of the invention to a SARS-COV-2 Spike protein binding molecule comprising at least one immunoglobulin single variable domain, which has at least one of the following characteristics:
i. binding to the SARS-COV-2 Spike protein; and
ii. inhibiting the infection and amplification of SARS-COV-2 in tissue culture.

The term “immunoglobulin single variable domain” is to be understood as referring to the variable region of a heavy chain comprised in an antibody. In a preferred embodiment, the antibody is a llama-derived antibody.

In a particular embodiment, the SARS-COV-2 Spike protein binding molecule is a single domain antibody. The term “single domain antibody” refers to an antibody which consists solely of the variable region of a heavy chain of an antibody. Within this description, the terms “single domain antibody”, “nanobody” and “VHH antibody” are used interchangeably. Preferably, the SARS-COV-2 Spike protein binding molecule is a llama-derived nanobody.

Llama-derived nanobodies have small molecular weights of only about 13-15 kDa, a diameter of about 2.5 nm and a length of about 4 nm. They represent the smallest molecule present in nature possessing an antigen-binding function.

In a particular embodiment, the SARS-COV-2 Spike protein binding molecule is a single domain antibody having at least 80% sequence identity to any one of SEQ ID NOs: 54 to 106, more preferably, at least 90% sequence identity to any one of SEQ ID NOs: 54 to 106, even more preferably, at least 99% sequence identity to any one of SEQ ID NOs 54 to 106. Preferably, the SARS-COV-2 Spike protein binding molecule is a single domain antibody having at least 80% sequence identity to any one of the group consisting of SEQ ID NOs: 67, 68, 90, 95, 98, 100 and 105, more preferably, at least 90% sequence identity to any one of the group consisting of SEQ ID NOs: 67, 68, 90, 95, 98, 100 and 105, even more preferably, at least 99% sequence identity to any one of the group consisting of SEQ ID NOs: 67, 68, 90, 95, 98, 100 and 105. In a particularly preferred embodiment, the SARS-COV-2 Spike protein binding molecule is a single domain antibody having at least 80% sequence identity to any one of the group consisting of SEQ ID NOs: 67, 68, 90 and 95, more preferably, at least 90% sequence identity to any one of the group consisting of SEQ ID NOs: 67, 68, 90 and 95, even more preferably, at least 99% sequence identity to any one of the group consisting of SEQ ID NOs: 67, 68, 90 and 95.

In another embodiment, the SARS-COV-2 Spike protein binding molecule is a single domain antibody having one or more amino acid substitutions, preferably conservative amino acid substitutions, compared with any one of SEQ ID NO: 54 to 106. Preferably, the SARS-COV-2 Spike protein binding molecule is a single domain antibody having one or more amino acid substitutions, preferably conservative amino acid substitutions, compared with any sequence of the group consisting of SEQ ID NOs: 67, 68, 90, 95, 98, 100 and 105. More preferably, the SARS-CoV-2 Spike protein binding molecule is a single domain antibody having one or more amino acid substitutions, preferably conservative amino acid substitutions, compared with any sequence of the group consisting of SEQ ID NOs: 67, 68, 90 and 95.

In a preferred embodiment, the SARS-COV-2 Spike protein binding molecule is a single domain antibody having an amino acid sequence as set forth in any of SEQ ID NO: 54 to SEQ ID NO: 106. More preferably, the SARS-COV-2 Spike protein binding molecule is a single domain antibody having an amino acid sequence selected from the group consisting of SEQ ID NOs: 67, 68, 90, 95, 98, 100 and 105. Even more preferably, the SARS-COV-2 Spike protein binding molecule is a single domain antibody having an amino acid sequence selected from the group consisting of SEQ ID NOs: 67, 68, 90 and 95.

In a particularly preferred embodiment, the SARS-COV-2 Spike protein binding molecule is a single domain antibody having an amino acid sequence as set forth in SEQ ID NO: 67.

It is yet another aspect of the present invention to provide a method for preparing a SARS-COV-2 Spike protein binding molecule according to the invention, comprising screening a phage library.

Screening a phage library involves a series of steps which are encompassed by this aspect of the present invention. Therefore, in a particular embodiment of the invention, screening a phage library comprises:
i. doing a series of biopannings with the prefused locked Spike protein and RBD domain; and
ii. conducting phage ELISAs and periplasmic extract ELISAs with the target antigens to confirm their recognition by the SARS-COV-2 Spike protein binding molecule of the invention.

According to the invention, the method for preparing a SARS-COV-2 Spike protein binding molecule additionally comprises:
iii. performing a sequencing analysis on the SARS-COV-2 Spike protein binding molecules identified in item ii.;
iv. transforming expression bacteria with the DNA sequence determined in item iii.; and
V. expressing and purifying the corresponding SARS-COV-2 Spike protein binding molecule.

However, a person of skill in the art will appreciate that the SARS-COV-2 Spike protein binding molecule of the invention can be obtained by any other technique known to a person of skill in the art for producing proteins, such as chemical synthesis.

The invention also provides an immunoconjugate comprising a SARS-COV-2 Spike protein binding molecule of the invention conjugated with one selected from the group consisting of a therapeutic moiety, an enzyme, a fluorochrome, and a human Fc.

Compared to the state of the art, the SARS-COV-2 Spike protein binding molecule provided by the present invention has the advantage of specifically binding to SARS-COV-2-Spike protein.

The SARS-COV-2 Spike protein binding molecule of the present invention can effectively avoid the binding of SARS-COV-2 Spike protein carried in pseudotyped lentiviruses and the ACE2 receptor expressed in transfected cells, as will be shown in the Examples.

The SARS-COV-2 Spike protein binding molecule of the invention is further able to block the infection process of SARS-COV-2 in cells, and inhibit the infection and amplification of two different strains of SARS-COV-2 in VERO cells.

The SARS-COV-2 Spike protein binding molecule provided by the invention also has a good binding specificity with SARS-COV-2 Spike protein, a high biological activity and stability and no toxic or side effect.

The person of skill in the art will appreciate that the scope of the invention encompasses obvious functionally active variants of SARS-COV-2 Spike protein binding molecules provided herein. By “functionally active variants”, one would understand, for instance, one of the following:
a. a polypeptide obtained by substituting and/or deleting and/or adding 1 or 2 or 3 amino acid residues to the amino acid sequence of a SARS-COV-2 Spike protein binding molecule of the invention, and having the same function;
b. a polypeptide having 99% or more, 95% or more, 90% or more, 85% or more, or 80% or more homology to the amino acid sequence of a SARS-COV-2 Spike protein binding molecule of the invention, and having the same function;
c. a fusion protein obtained by connecting the N-terminus and/or the C-terminus of a SARS-COV-2 Spike protein binding molecule of the invention, where said label may be a Flag tag, a His tag, an MBP tag, an HA tag, a myc tag, a GST tag, and/or a SUMO tag, among others.

It is another aspect of the invention to provide a nucleic acid molecule encoding a SARS-COV-2 Spike protein binding molecule according to the invention.

The term “nucleic acid molecule” is to be understood broadly. For instance, the nucleic acid molecule of the invention may be an RNA molecule, a DNA molecule, or other nucleic acid molecules which can be obtained by artificial synthesis or isolated from proper natural sources.

In a particular embodiment, the nucleic acid molecule has a nucleotide sequence having 80% or more or 90% or more homology to a nucleotide sequence as set forth in any of SEQ ID NO: 1 to SEQ ID NO: 53. Preferably, the nucleic acid molecule has a nucleotide sequence as set forth in any of SEQ ID NO: 1 to SEQ ID NO: 53. More preferably, the nucleic acid molecule has a nucleotide sequence selected from the group consisting of SEQ ID NOs: 14, 15, 37, 42, 45, 47, and 52. Even more preferably, the nucleic acid molecule has a nucleotide sequence selected from the group consisting of SEQ ID NOs: 14, 15, 37 and 42. Most preferably, the nucleic acid molecule has a nucleotide sequence as set forth in SEQ ID NO: 14.

A person of skill in the art will appreciate that the nucleic acid of the invention may be used in appropriate vectors to transform cells so that said cells are able to express a SARS-COV-2 Spike protein binding molecule of the invention.

Therefore, the present invention encompasses within its scope an expression cassette, a recombinant vector, a recombinant bacterium, or a transgenic cell line comprising a nucleic acid molecule of the invention.

It is yet another aspect of the invention to provide a pharmaceutical composition comprising a SARS-COV-2 Spike protein binding molecule according to the invention, or an immunoconjugate thereof, or both.

The person of skill in the art will appreciate that a pharmaceutical composition according to the invention may comprise a pharmaceutically acceptable carrier, as well as pharmaceutically acceptable excipients. It is within the expected knowledge of a person of skill in the art to select an appropriate pharmaceutically acceptable carrier and appropriate pharmaceutically acceptable excipients to obtain a pharmaceutical composition with the intended performance.

The inventors have found the combination of more than one SARS-COV-2 Spike protein binding molecule of the invention generates synergetic effects in the capability of said molecules to bind to the Spike protein. Therefore, it is also an aspect of this invention to provide a pharmaceutical composition comprising at least two SARS-COV-2 Spike protein binding molecules according to the invention. In a preferred embodiment of this aspect of the invention, the pharmaceutical composition comprises a SARS-COV-2 Spike protein binding molecule having the amino acid sequence set forth in SEQ ID NO: 67 and a SARS-COV-2 Spike protein binding molecule having the amino acid sequence set forth in SEQ ID NO: 68. In another preferred embodiment of this aspect of the invention, the pharmaceutical composition comprises a SARS-COV-2 Spike protein binding molecule having the amino acid sequence set forth in SEQ ID NO: 95 and a SARS-COV-2 Spike protein binding molecule having the amino acid sequence set forth in SEQ ID NO: 105.

In a particular embodiment of this aspect of the invention, the pharmaceutical composition comprises three different SARS-COV-2 Spike protein binding molecules. Preferably, pharmaceutical composition comprises a SARS-COV-2 Spike protein binding molecule having the amino acid sequence set forth in SEQ ID NO: 67, a SARS-COV-2 Spike protein binding molecule having the amino acid sequence set forth in SEQ ID NO: 68, and a SARS-COV-2 Spike protein binding molecule having the amino acid sequence set forth in SEQ ID NO: 95.

As described, SARS-COV-2 Spike protein binding molecule of the invention has the property of binding specifically to the SARS-COV-2 Spike protein and avoiding its binding with human ACE2 receptor.

Therefore, it is another aspect of the invention to provide a method for method for preventing and/or treating a disease caused by infection by novel coronavirus SARS-COV-2, comprising administering a therapeutically effective amount of a SARS-COV-2 Spike protein binding molecule or a pharmaceutical composition according to the invention to a subject in need thereof.

It is also an aspect of the invention to provide a use of a SARS-COV-2 Spike protein binding molecule according to the invention for the manufacture of a medicament for preventing and/or treating a disease caused by infection by novel coronavirus SARS-COV-2.

In a particular embodiment, preventing and/or treating a disease caused by infection by novel coronavirus SARS-COV-2 comprises inhibiting the infection of a subject by novel coronavirus SARS-COV-2.

Preferably, the disease caused by the infection by novel coronavirus SARS-COV-2 is selected from the group consisting of human novel coronavirus pneumonia, intestinal disease, intra vascular disseminated coagulation, and encephalitis.

The SARS-COV-2 Spike protein binding molecule of the invention can also be used for other ends involving the binding thereof to a SARS-COV-2 Spike protein.

Therefore, it is yet another aspect of the invention to provide a use of a SARS-COV-2 Spike protein binding molecule according to the invention as a diagnosis reagent.

It is also an aspect of the invention to provide a method to diagnose an infection by SARS-COV-2 in a subject, which comprises contacting a sample obtained from the subject with a SARS-COV-2 Spike protein binding molecule according to the invention, and detecting the binding of the SARS-COV-2 Spike protein binding molecule to the SARS-COV-2 by an appropriate technique. It is within the knowledge expectable for any person of skill in the art to select a technique to detect the binding.

It is yet another aspect of the invention to provide a use of a SARS-COV-2 Spike protein binding molecule according to the invention as a capture reagent in the Spike purification process or as a concentrator of low amount of virus in a sample. In a particular embodiment, the sample is selected from the group consisting of food, water, feces, blood, serum and body secretions.

### EXAMPLES

The present invention will be described in further detail with reference to the following Examples. The invention is not limited to the examples given. The methods used are conventional methods unless otherwise specified, and the reagents and materials used are commercially available products unless otherwise specified.

### Example 1-Llama Immunization and Monitoring of Immune Response by ELISA

Mammalian expression plasmids encoding for SARS-COV-2 S-2P protein and polypeptide corresponding of the Receptor Binding Domain (RBD) containing N-terminal secretion signal sequences and C-terminal 6xHis tag were transfected into HEK-293 cells using polyethylenimine (PEI). Supernatants were harvested 72 hours post-transfection, filtered through a 0.22 micrometer membrane and incubated with nickel affinity resin (Amintra) according to the manufacturer instructions. Proteins were purified using Poly-Prep Chromatography Columns (Bio-Rad) and eluted by Imidazole.

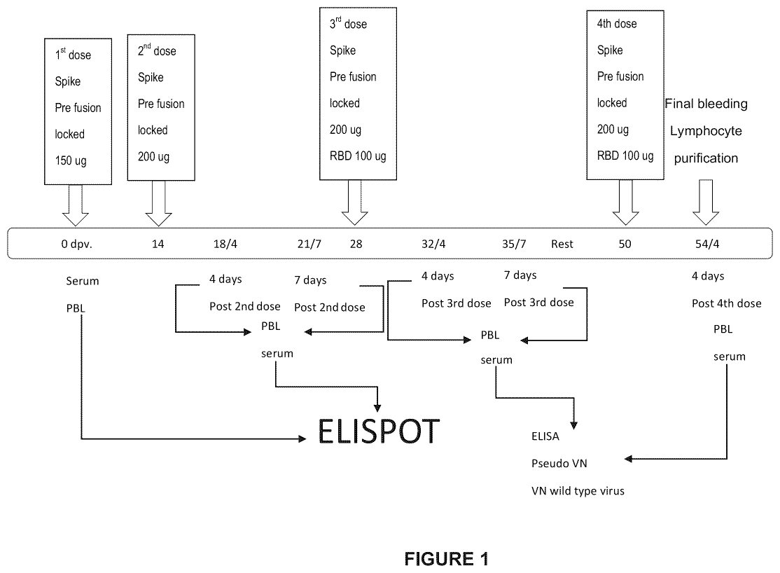

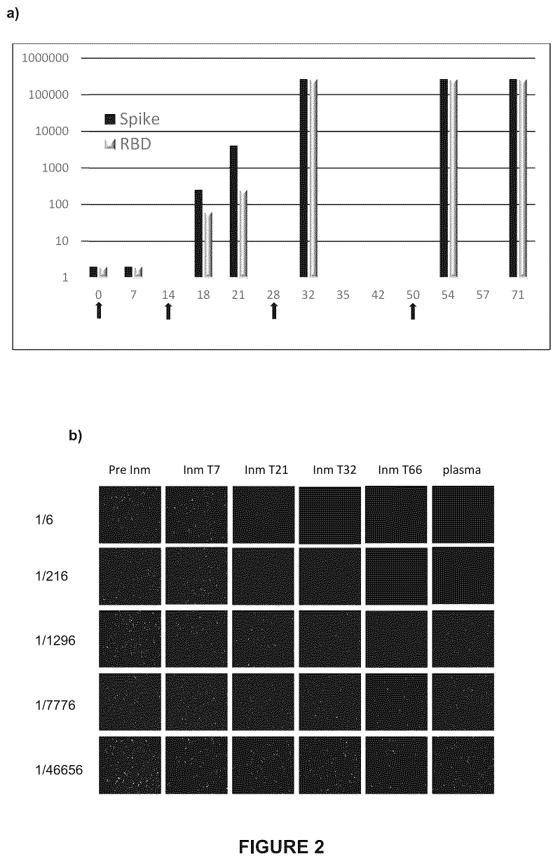

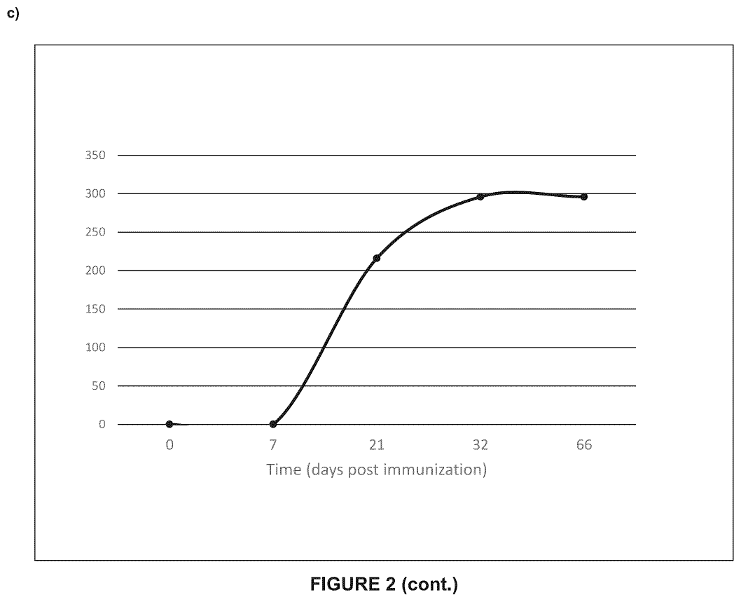

A male llama, younger than one year of age, was immunized with the S protein of the SARS-COV-2 virus and with the Receptor Binding Domain (RBD) of the SARS-COV-2 protein S. A total of 4 doses were applied with complete Freund's Adjuvant in the first dose and incomplete in the following (FIG. 1). At day 54 after starting the immunization process (4 days after the last dose), the final bleeding was conducted. The lymphocyte extraction was performed from 200 ml of llama blood. During the llama immunizations, serum samples were taken to monitor the llama's antibody response. Antibodies against coronavirus protein S and RBD used in llama immunizations were measured by ELISA and virus neutralization (VN) using pseudo particles. The immunized llama reached an anti-S and anti-RBD IgG antibody titer of 262, 144 and a VN Ab titer of 296 (FIG. 2).

### Example 2-VHH Gene Library Construction

For the construction of the gene library, the Golden-Gate system and the pmecs GG phagemid were used following procedures previously described (Pardon et al., A general protocol for the generation of Nanobodies for structural biology (2014), 674, VOL.9 NO.3, Nature Protocols; Serge Muyldermans, A guide to: generation and design of nanobodies, The FEBS Journal (2020)). From the llama's lymphocytes, 1×10⁶ cells, total RNA was extracted using a RNAeasy Midi Kit (Qiagen). Subsequently, a reverse transcription reaction was performed to produce the copy DNA with oligo dT primer. The VHH sequences were amplified from the cDNA pool with a series of two PCRs using the following primers:

CALL001:
(SEQ ID NO: 107)
5′-GTCCTGGCTGCTCTTCTACAAGG-3′,
CALL002:
(SEQ ID NO: 108)
5′-GGTACGTGCTGTTGAACTGTTCC-3′
for the first round and
VHH-Back-SAPI:
(SEQ ID NO: 108)
5′-CTTGGCTCTTCT∨GTG∧CAGCTGCAGGAGTCTGGRGGAGG-3′
and
VHH-Fw-SAPI:
(SEQ ID NO: 110)
5′-TGATGCTCTTCC∨GCT∧GAGGAGACGGTGACCTGGGT-3′
for the second reaction (Pardon et al.;
Muyldermans).

In a first reaction, two fragments were amplified, one of approximately 0.7 kb (corresponding to the heavy chain of antibodies formed only by heavy chain) and another of approximately 0.9 kb (corresponding to the heavy chain of conventional antibodies). The 0.7 kb fragment, which gave rise to the VHH sequences, was purified from a preparative agarose gel and then used as a template in nested PCR. This PCR also introduced the Sap I restriction enzyme sites at the 5′ and 3′ ends of the VHH amplicons.

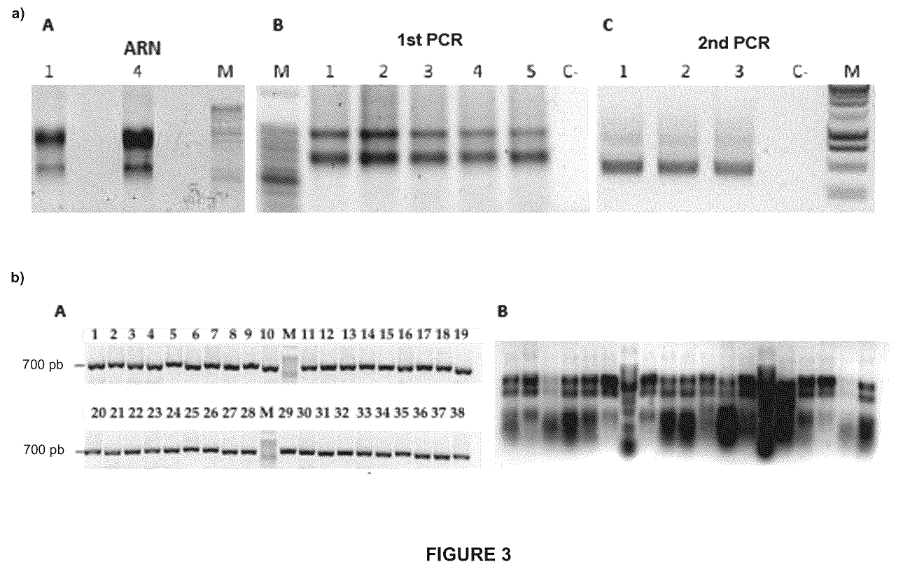

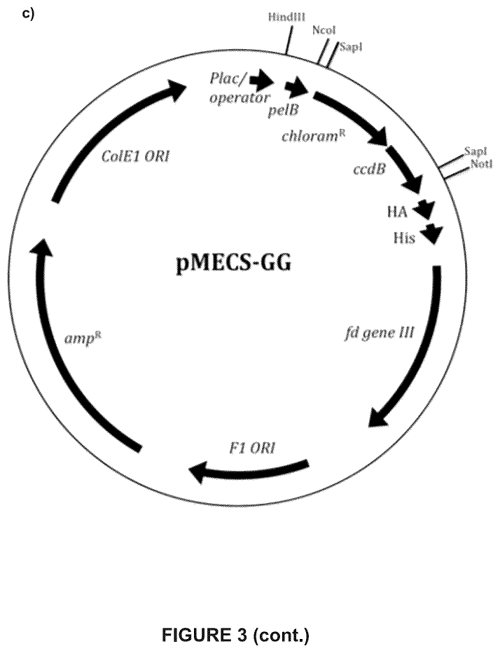

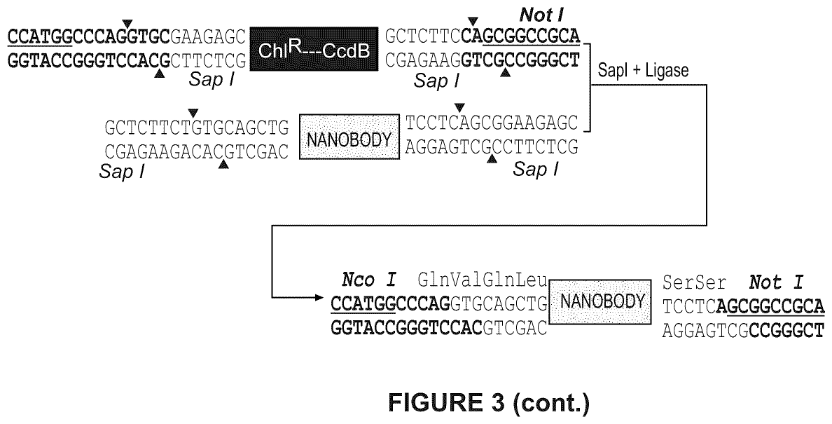

VHH encoding sequences were cloned between SapI sites of the phagemid vector pMECS-GG (FIG. 3) by using sequential ligation and restriction reactions. The vector pMECS-GG was obtained from a modification of the vector pMECS to which a “killer cassette” flanked by Sap I restriction enzyme sites within the multiple cloning site was introduced. In addition to a chloramphenicol resistance gene for positive selection in cells containing the CcdA gene, this killer cassette encodes the CcdB protein that will kill those TG1 cells that lack CcdA. Cells that survive are used to prepare phagemid DNA pMECS-GG. Mixing the phagemid pMECS-GG and the PCR amplicon followed by incubation with Sap I removes the enzyme recognition sequence from the amplicon and generates three protruding nucleotides. In the plasmid, the Sap I enzyme cuts the filler fragment containing the chloramphenicol and ccdB genes, the remaining phagemid will have three protruding nucleotides that are complementary to the protruding nucleotides in the cut VHH PCR amplicon. Ligation of this chimeric pMECS to the VHH gene will create stable chimeric phagemids without SapI recognition sequence that will be transformed into the bacterium TG1.

For the library construction, electro-competent E.coli TG1 cells [Genotype TG1: K-12 glnV44 thi-1 Δ (lac-proAB) Δ (mcrB-hsdSM)5(rK-mK-) F′ [traD36 proAB+Iaclq IacZΔM15] were transformed resulting in a VHH library of about 1.8×10⁹ independent colonies that were resuspended in LB medium and divided in 24 tubes of 2 ml each, and stored at −80 and −196° C. For conducting the library's screening, 2 ml of bacteria from the stock library were grown in 2xTY medium and when OD600 0.6 was reached they were infected with VCS M13 helper phages in order to expand the VHH phagemic library and use it for the selection of specific VHHs to the proteins of interest: pre-fused and locked Spike and the soluble polypeptide corresponding to the ACE2 receptor binding domain, RBD.

### Example 3-Biopanning of VHH Library with Spike Protein and RBD. Phage ELISA and Periplasmic Extract ELISA

Biopanning of the generated library was performed using as a strategy the direct coating of RBD and S proteins into a 96-well ELISA plate. Briefly, microtiter ELISA plates (Maxisorp-Thermo Scientific) were coated overnight at 4° C. with 10 ug/well of SARS-COV-2 recombinant protein Spike or RBD. Uncoated wells were used as a negative control. The day after, plates were washed 3 times with PBST (phosphate buffered saline pH7+0.05% Tween 20) and blocked with 1% skim milk in PBST at 37° C. for 1 h. Approximately 1000 phages in 100 ul of blocking buffer were added and incubated for 2 hs at room temperature with shaking. In the first panning round wells were washed 10 times with PBST, while 5 washing steps were used for the second and third rounds. The remaining phages were eluted with TEA-solution (14% trimethylamine 371 (Sigma) pH 10) treatment for 5 min following by neutralization with 1 M Tris-HCl PH 8 solution. After each panning round phages were amplified by infection of exponentially growing E. coli TG1 cells and superinfected with VCS M13 helper phages. After centrifugation to remove glucose, bacteria were grown over night in the presence of ampicillin and kanamycin antibiotics. The next day phages were purified using PEG 6,000/NaCl precipitation and used for the next round of selection.

### PE-ELISA and Phage ELISA

Periplasmic extracts and recombinant phages from individual colonies were tested for binding to either SARS-COV-2 2P or RBD protein. Wells of a microtiter plate (Maxisorp) were coated overnight at 4° C. with 10 ug in 10 ml of Spike, RBD of SARS.CoV-2 or irrelevant protein as negative controls. After washing with PBST, wells were blocked with 1% skim milk powder in PBST and 50 μL of the periplasmic extract or diluted recombinant phages was added to the wells. For PE-ELISA VHH specific binding was detected with homemade polyclonal rabbit sera (1/5000) followed by horseradish peroxidase (HRP)-linked anti-rabbit IgG (1/3000). In the case of Phage ELISA anti M13-HRP (Invitrogen) was used in a 1/5000 dilution. After washing 50 μL of ABTS/H₂O₂ substrate was added to the plates and the reaction was stopped by addition of 5% SDS. The absorbance at 405 nm was measured with an Multiskan ELISA reader. To determine specific binding, an OD450 value of the antigen coated wells at least two times higher than the OD450 value of the control wells were considered as positive.

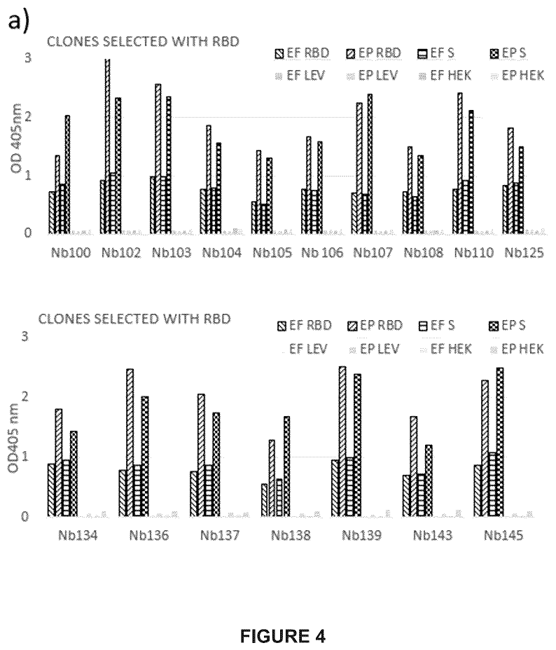

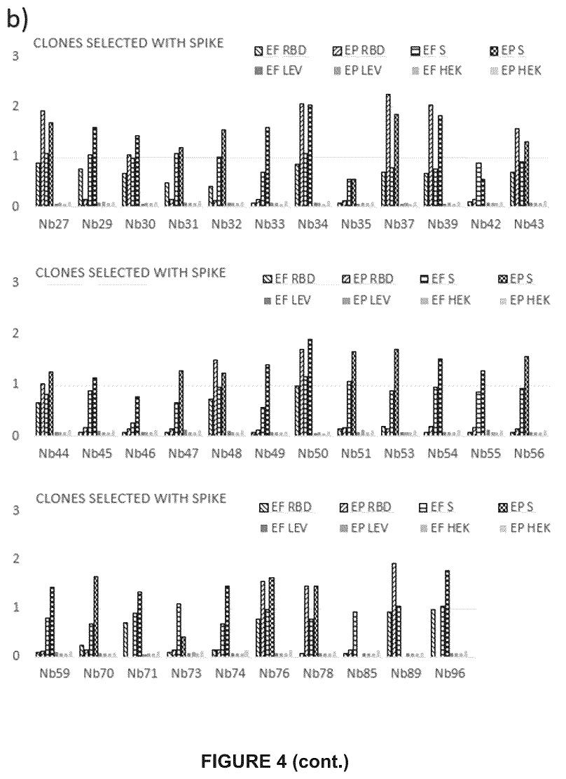

Considering the results of both ELISA assays, a total of 17 wells containing phages of soluble VHH fragments (nanobodies fused to the pill protein of the phage) were retrieved in the biopanning conducted that recognized RBD alone or in the context of the Spike protein, while 34 positive clones were retrieved in the biopanning conducted with Spike (FIG. 4).

### Example 4-Analysis with Restriction Enzymes and DNA Sequencing of Nanobodies Encode Sequences

All the nanoantibody sequences that reacted in the phage ELISA and in the periplasmic extract ELISA were selected and cloned into DH5α E. coli for subsequent analysis with restriction enzymes. Plasmids from positive clones were transformed in DH5α bacteria and further characterized by restriction with Hinfl before sending samples for sequencing.

The different restriction profiles were selected for further sequencing analysis and protection of the DNA and aa sequences of each nanoantibody. From these analyses, a total of 50 different unique sequences were obtained and are preferred embodiments of the present invention. See list of nt and aa listed below.

### Example 5-Transformation of WK6 Expression Strain of E coli, Nanobody Expression and Purification by Immobilized Metal-ion Affinity Chromatography (IMAC) and Size Exclusion Chromatography (SEC)

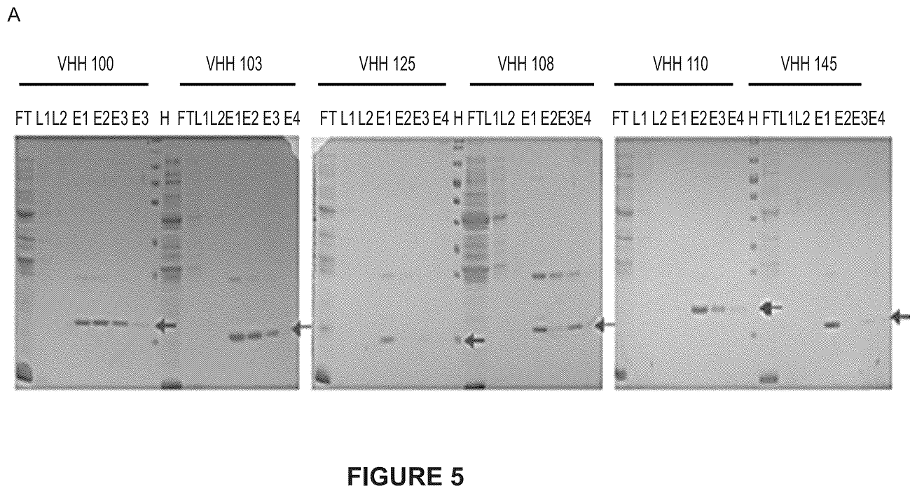

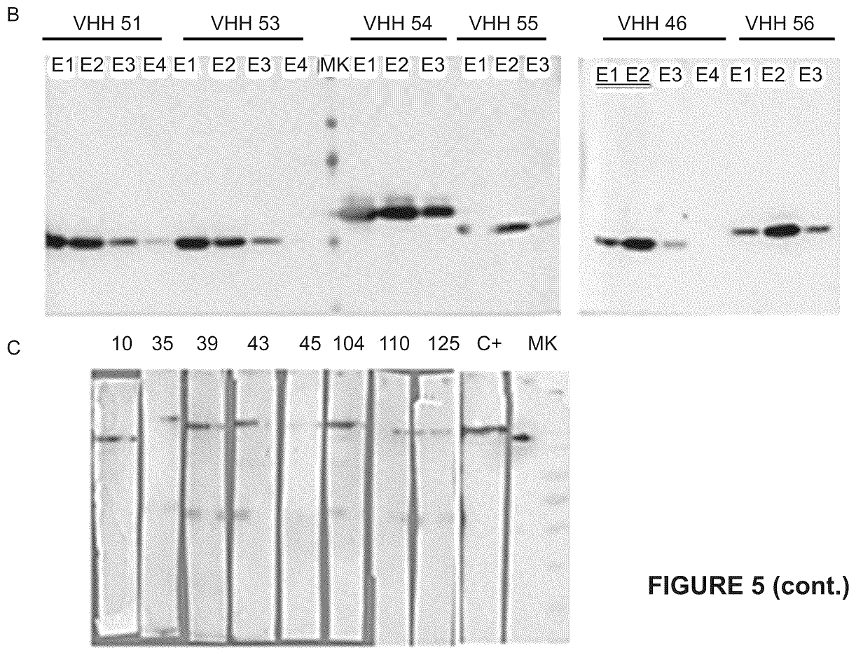

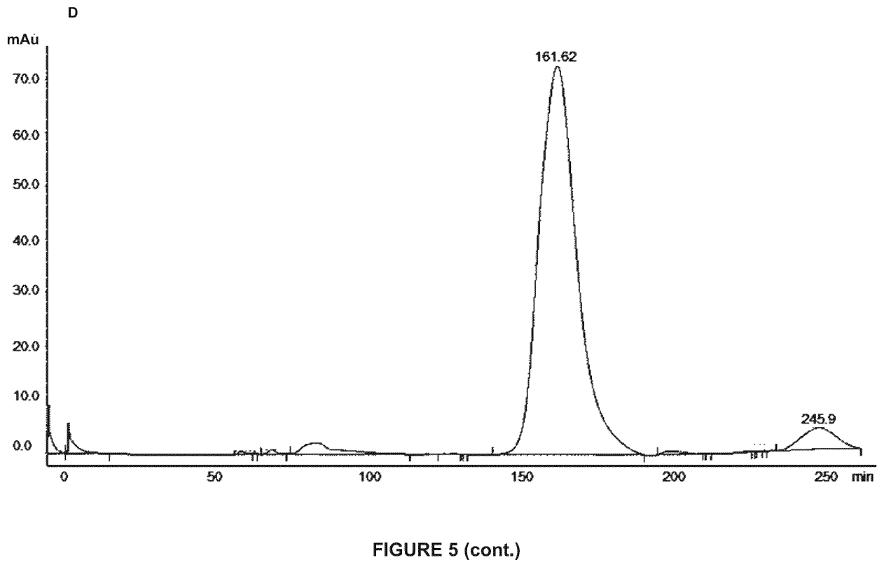

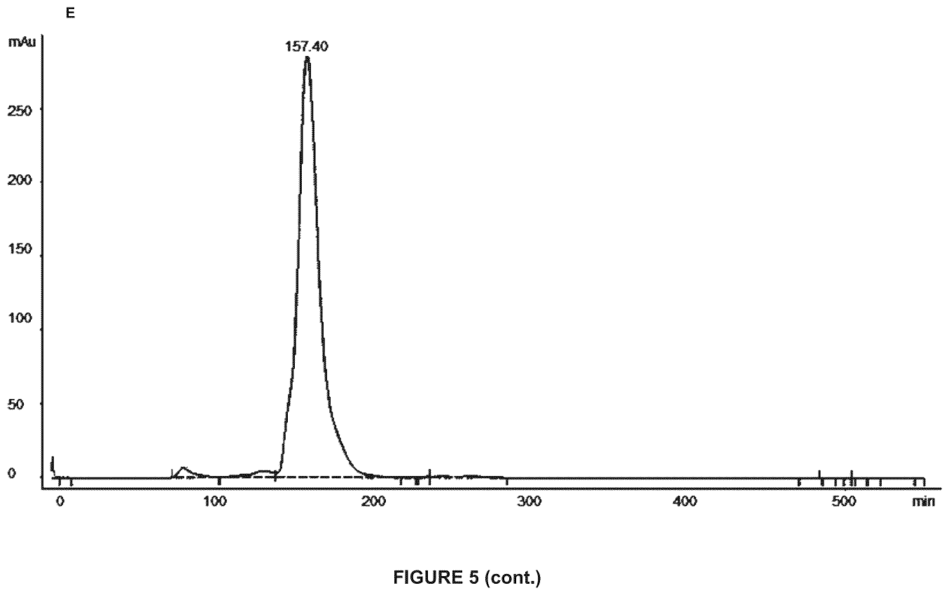

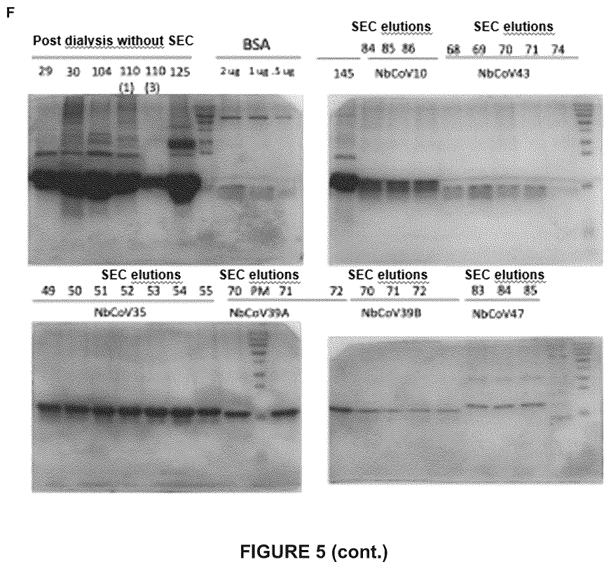

The 50 phagemid vectors containing the sequences referred to in Example 4 were cloned into WK6 E. coli genotype: F′ Iaclq Δ (IacZ) M15 proA+B+Δ (IacproAB) galE rpsL for further nanobody expression and purification by IMAC and SEC (FIG. 5). The WK6 strain is able to read the “Amber” stop codon present in the pMECsGG phagemid, then generating the nanoantibody free of the phage protein. The present of the pelB signal directs the expression to the periplasmic space.

The bacterial pellets obtained from the expression cultures were processed to obtain the content of the periplasmic space. For this, they were resuspended in 6 ml of TES buffer (200 mM Tris, 500 μM EDTA, 500 mM Sucrose pH=8.5) and frozen in liquid nitrogen. After thawing the extracts, an osmotic shock was carried out by adding 7.5 ml of a dilution to the quarter of the TES and incubating for 1 hour on ice. The extracts were centrifuged at 30,000 g for 20 minutes, the supernatants were filtered with a syringe by 0.2 μm and diluted with an equal volume of 100 mM Tris buffer pH 7.0 added with 4 mM MgCl2, 1 M NaCl, Imidazole 20 mM. The nanoantibodies present in these extracts were separated from them by IMAC using a Ni²⁺ cation-bound sepharose matrix present in HiTrap IMAC columns (GEHealthcare #Cat.: 17-5247-05). The six-histidine tag present in the nanoantibodies allows their binding by coordination to the aforementioned cation, thus remaining trapped in the matrix while the rest of the components of the extract pass by or are washed in the successive steps of chromatography. Finally, the nanoantibodies were eluted from the resin using imidazole at a concentration of 0.5 to 1.0 M. As a final step in purification and with the aim of eliminating the imidazole and changing the buffer in which they are found; nanoantibodies were dialyzed against PBS within semipermeable membranes of MWCO from 6 KDa to 8 KDa (Spectrum™ #Cat.: 132660). The pure nanoantibodies were stored at −80° C. until use. The production and purification process was monitored by SDS-PAGE. In the scaling up process the nanobodies were expressed in 4-liters bioreactor (Biostat B Plus twin, Sartorius) and purified using an Akta Pilot chromatographer for the IMAC step and an Akta prime for the SEC.

The nanobodies were expressed with variable yields obtaining purified stocks from 0.1 mg/ml to 10 mg/ml depending on the clone expressed (see Table 1).

TABLE 1
Yield of the expressed nanobodies

| | | Yield | |
|---|---|---|---|
| | | (mg of | |
| | | protein/liter | |
| Nanobody ID | Batch record | of culture) | MW |
| 10 Mebus | 400 ml --> 374 ug | 0.9 | 17945.16 |

Nabobodies selected with Spike

| | | | |
|---|---|---|---|
| 27 | 500 ml --> 160 ug | 0.3 | 17336.46 |
| 29 | 200 ml --> 200 ug | 1 | 17798.79 |
| 30 | 500 ml --> 38 ug | 0.07 | 17376.43 |
| 31 | 200 ml --> 35 ug | 0.17 | 17948.75 |
| 32 | 200 ml --> 700 ug | 3.5 | 17827.39 |
| 33 | 200 ml --> 408 ug | 2 | 18330.03 |
| | 600 ml --> 311 ug | 0.5 | |
| | 1300 ml --> 2500 ug | 1.9 | |
| | 1400 ml --> 5 mg | 3.5 | |
| 34 | | | 16289.00 |
| 35 | 1300 ml --> 5000 ug | 3.8 | 18031.77 |
| | 1400 ml --> 18 mg | 12.8 | |
| 37 | 200 ml --> 452 ug | 2.2 | 17497.60 |
| 39A | 900 ml --> 3000 ug | 3.3 | 18139.11 |
| 39B | 600 ml --> 2800 ug | 4.6 | |
| 40 | 250 ml --> 500 ug | 2 | 17857.80 |
| 42 | | | |
| 43 | 1000 ml --> 1800 ug | 1.8 | 17577.92 |
| | 1400 ml --> 1670 ug | 1.2 | |
| 44 | 900 ml --> 1700 ug | 1.9 | 16627.62 |
| 45 | 800 ml --> 8 mg | 10 | 17747.54 |
| 46 | 800 ml --> 1240 ug | 1.5 | 18581.14 |
| 47 | 800 ml --> 2500 ug | 3.1 | 16595.45 |
| 48 | 800 ml --> 1250 ug | 1.5 | |
| 49 | 800 ml --> 500 ug | 0.6 | 16096.21 |
| 50 | | | 15653.23 |
| 51 | | | |
| 53 | | | 17655.78 |
| 54 | | | 17910.87 |
| 55 | | | |
| 56 | | | 18707.26 |
| 59 | | | |
| 70 | | | |
| 71 | 200 ml --> 20 ug | 0.1 | 18151.24 |
| 73 | 200 ml --> 900 ug | 4.5 | 18224.32 |
| 74 | | | |
| 76 | | | |
| 78 | 200 ml --> 800 ug | 4 | 18359.57 |
| 85 | | | 17857.80 |
| 87 | | | |
| 89 | | | 16012.59 |
| 96 | 400 ml --> 447 ug | 1.1 | 15645.51 |

Nanobodies selected with RBD

| | |
|---|---|
| 100 | 15848.36 |
| 102 | 17346.59 |
| 103 | 17306.48 |
| 104 | 15131.67 |
| 105 | 16259.82 |
| 106 | 15107.69 |
| 107 | 15919.54 |
| 108 | 14777.38 |
| 110 | 16246.78 |
| 125 | 16888.90 |
| 134 | |
| 136 | 18122.13 |
| 137 | |
| 138 | |
| 139 | |
| 143 | 15290.90 |
| 145 | 16089.77 |

### Example 6-Functional Characterization. Binding Affinity to the Target Antigen and Estimation of the Median Effective Dose (EC50) by ELISA

Ninety-six ELISA wells were coated with 1 ug/ml of Spike or RBD protein in a carbonate/bicarbonate buffer, pH: 9.6, and incubated overnight at 4° C. After a blocking step with 1% skim milk in 0.05% PBST, the purified nanobodies were adjusted to a concentration of 1000 nM and assayed in 10-fold dilution on each plate. A rabbit polyclonal antibody anti-VHH was added in a 1/2000 dilution, followed by a commercial goat anti rabbit IgG (Jackson Immuno Research) labeled with HRP in a 1/5000 dilution. Every step was followed by 4 washes with 0.05% PBST. All steps were incubated at 37° C. and 5% CO₂. The reaction was developed with H₂O₂/ABTS as substrate/chromogen system, and stopped with 5% SDS. Optical density (OD) was measured at 405 nm. The EC50 was estimated using a four-parameter logistic regression model (AAT Bioquest, Inc. (2020, Nov. 14). Quest Graph™ IC50 Calculator.″. Retrieved from https://www.aatbio.com/tools/ic50-calculator)

TABLE 2
Summary results of nanobody reactivity with Spike
and RBD in different ELISA and estimation of EC50

| | | BIOPANNING | BIOPANNING | | |
|---|---|---|---|---|---|
| | | Phage ELISA / | Phage ELISA / | | |
| | | PERIPLASMIC | PERIPLASMIC | EC50 | EC50 |
| | | EXTRACT | EXTRACT | nM | nM |
| Nanobody | BIOPANNING | ELISA | ELISA | ELISA | ELISA |
| ID | ANTIGEN | Spike | RBD | Spike | RBD |
| 27 | Spike | Positive / | Positive / | 0.35 | 0.7 |
| | | Positive | Positive | | |
| 29 | Spike | Positive / | Positive / | 0.05 | 55.89 |
| | | Positive | Negative | | |
| 30 | Spike | Positive / | Positive / | 5.84 | negative |
| | | Positive | Positive | | |
| 31 | Spike | Positive / | Positive / | ND | 137.42 |
| | | Positive | Negative | | |
| 32 | Spike | Positive / | Negative / | 0.012 | 83.6 |
| | | Positive | Negative | | |
| 33 | Spike | Positive / | Negative / | 0.025 | nd |
| | | Positive | Negative | | |
| 35 | Spike | Positive / | Negative / | 23.77 | nd |
| | | Positive | Negative | | |
| 37 | Spike | Positive / | Positive / | 3.36 | 0.04 |
| | | Positive | Positive | | |
| 39A | Spike | Positive / | Positive / | 0.14 | 0.29 |
| | | Positive | Positive | | |
| 40 | Spike | Positive / | Negative / | 1.06 | 169 |
| | | Positive | Negative | | |
| 43 | Spike | Positive / | Positive / | 0.045 | 0.18 |
| | | Positive | Positive | | |
| 44 | Spike | Positive / | Positive / | 50.44 | 16.75 |
| | | Positive | Positive | | |
| 45 | Spike | Positive / | Negative / | 0.42 | nd |
| | | Positive | Negative | | |
| 46 | Spike | Positive / | Negative / | 0.38 | nd |
| | | Positive | Negative | | |
| 48 | Spike | Positive / | Positive / | 0.56 | 1.78 |
| | | Positive | Positive | | |
| 71 | Spike | Positive / | Positive / | nd | 1.63 |
| | | Positive | Positive | | |
| 78 | Spike | Positive / | Negative / | 0.64 | 16.59 |
| | | Positive | Positive | | |
| 85 | Spike | Positive / | Negative / | 0.81 | nd |
| | | Positive | Negative | | |
| 89 | Spike | | | 14.65 | + |
| 100 | RBD | Positive / | Positive / | 1.92 | neg |
| | | Positive | Positive | | |
| 104 | RBD | Positive / | Positive / | 12.42 | 5.45 |
| | | Positive | Positive | | |
| 105 | RBD | Positive / | Positive / | 1.08 | 4.7 |
| | | Positive | Positive | | |
| 106 | RBD | Positive / | Positive / | 3.79 | 10.52 |
| | | Positive | Positive | | |
| 108 | RBD | Positive / | Positive / | + | neg |
| | | Positive | Positive | weak | |
| 110 | RBD | Positive / | Positive / | + | 32.41 |
| | | Positive | Positive | | |
| 125 | RBD | Positive / | Positive / | 12.04 | nd |
| | | Positive | Positive | | |
| 136 | RBD | Positive / | Positive / | 175 | 13.45 |
| | | Positive | Positive | | |
| 145 | RBD | Positive / | Positive / | 10.65 | 4.63 |
| | | Positive | Positive | | |

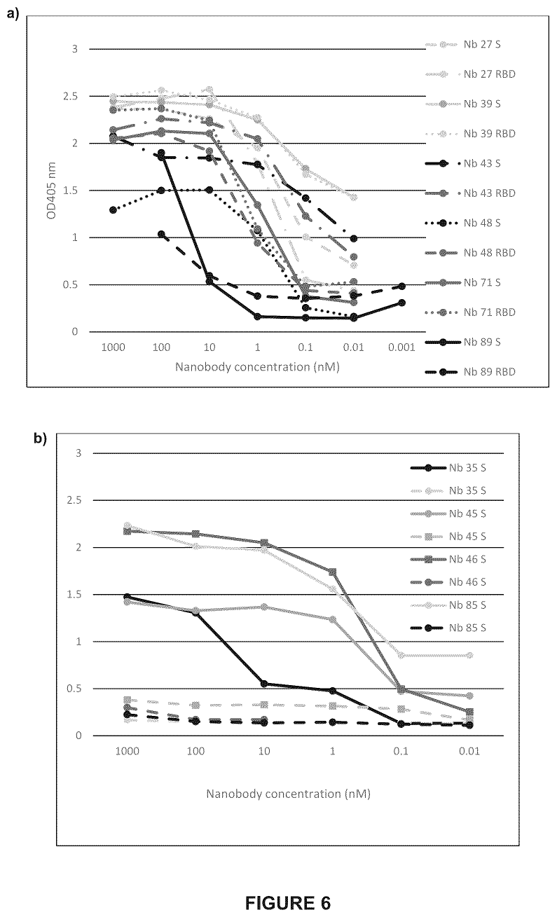

Table 2 summarizes the nanobodies that were selected using Spike or RBD in the biopanning that, when expressed in WK6 E coli in their soluble form, were able to react with their target antigens by ELISA, RBD alone or in the context of Spike or only Spike. All nanobodies tested showed EC50 in the nanomolar range. Clones 27, 37, 39A, 39B, 43, 44, 48,71 selected in the biopanning with Spike were directed to RBD, while the clones 32, 33, 35, 40, 45, 46, and 85 were directed to epitopes outside RBD (FIG. 6, Table 2). As expected, all of the nanobodies retrieved from the biopanning using RBD recognized RBD in its soluble form or in the context of the full Spike pre-fused and locked.

### Example 7-Functional Characterization Neutralization Activity

The neutralizing activity was determined using pseudotyped lentiviral particles expressing the Spike protein of the Wuhan strain, and wildtype virus circulating in Argentina and North America

Treatment of samples: prior to be tested, all nanobodies were subjected to a heat bath treatment at 56° C. for 30 minutes, then centrifuged at 2000 rpm for 10 minutes. In the case of IMAC-SEC purified samples, they were not subjected to inactivation treatments.

Pseudoviruses expressing SARS-COV-2 Spike protein were produced by co-transfection of plasmids encoding a GFP protein, a lentivirus backbone, and Spike genes in HEK-293T cells. Serial dilutions of VHHs were mixed with the pseudoviruses, incubated for 2 h at room temperature, and then added to HEK-293T cells previously transfected with the ACE2 receptor. 72 hours later, the cells were observed under the microscope and GFP-positive cells were counted. Percent neutralization was calculated considering uninfected cells as 100% neutralization and cells transduced with only pseudovirus as 0% neutralization. Neutralizing end titers were expressed at the minimal concentration of nanobody showing 100% of neutralization of Spike transduction in the cells.

For the neutralization assays using the wild-type virus circulating in Argentina, nanobodies were tested at Malbran Institute and the Virology Institute, INTA.

Vero cells were seeded in 96-well plates with 1.5×10⁴ cells per well and incubated for 24 hours at 37° C. in 5% CO₂ atmosphere. The nanobodies were prepared in a 10,000 nM concentration and were serially 2-fold diluted in Eagle-Dulbecco media supplemented with 2% fetal calf serum, 1600 U/ml penicillin, 800 μg/ml streptomycin and 10 μg/ml amphotericin B. Nanobody dilutions were incubated with 1000 DICT50 of virus, at 37° C. for 1 hour in equal volumes. The assay conducted at Malbran Institute used a viral strain isolated from the sample of an Argentinean patient, such strain was characterized through the sequencing of its complete genome, the sequence was deposited in GenBank: CoV-19/Argentina/C121/2020/EPI_ISL_420600/2020-03-07. In the case of the VN assay performed at INTA, the virus was the isolate named B. 1.499, hCoV-19/Argentina/PAIS-C0102/2020 or Andina Strain. The mixtures of the nanobody dilutions-virus were added to plates with confluent Vero cells monolayers and incubated at 37° C., in 5% CO₂ atmosphere, for 1 h. Afterwards, inocula were removed from the wells, cells were washed with PBS pH 7.2 and incubated with D-MEM2% for 72 h. The occurrence of cytopathic effect (CPE) was examined daily under the microscope. The complete absence of CPE was defined as protection by comparison with virus, antibody and virus-free cell controls. In the case of the VN conducted at INTA, cells were fixed with 70% acetone and the virus replication confirmed by immunofluorescent staining using the purified IgG from the immunized llama labeled with FICT.

Criteria for reporting results: The result of the neutralizing power is reported as the titre of each sample calculated according to the inverse of the highest dilution that evidences absence of CPE and positive fluorescence, comparable to the control of Vero cells without virus.

If all dilutions of the antibody show neutralizing activity, the result should be interpreted as a higher titer in the inverse of the last dilution tested. If none of the sample dilutions evidence neutralizing activity, the result will be a lower titer than the inverse of the first dilution tested.

The neutralization test was a plaque reduction assay using the American Isolate USA-WA1/2020, that was isolated from an oropharyngeal swab from a patient with a respiratory illness who had recently returned from travel to the affected region of China and developed clinical disease (COVID-19) in January 2020 in Washington, USA. Under the nomenclature system introduced by GISAID (Global Initiative on Sharing All Influenza Data), SARS-COV-2, isolate USA-WA1/2020 is assigned lineage A and GISAID clade S using Phylogenetic Assignment of Named Global Outbreak LINeages (PANGOLIN) tool. The complete genome of SARS-COV-2, USA-WA1/2020 has been sequenced (the isolate-GenBank: MN985325 and GISAID: EPI_ISL_404895 and after one passage in Vero cells-GenBank: MT020880). The complete genome of SARS-COV-2, USA-WA1/2020 has been sequenced after four passages in Vero cells in collaboration with Database for Reference Grade Microbial Sequences (FDA-ARGOS; GenBank: MT246667). BioProject PRJNA716271, Sequencing of SARS-COV-2 Isolates from BEI Resources, has been published on the NCBI website. The sequences from the BEI Resources SARS-COV-2 isolates included in the project have been deposited into GenBank (SRA: SRP312598) and the sequence for SARS-CoV-2, isolate USA-WA1/2020 (NR-52281), is available (SAMN 18527778). Additional information and tools are available at ViPR (Virus Pathogen Resource). The strain was obtained through BEI Resources, NIAID, NIH: SARS-Related Coronavirus 2, Isolate USA-WA1/2020, NR-52281. The reagent was deposited in BEI by the Centers for Disease Control and Prevention.

TABLE 3
Summary results of neutralizing activity in pseudo VN and wildtype VN assays.

| | Wuhan | | CoV-19/Argentina/ | | CoV-20/Argentina/ | | USA- | |
|---|---|---|---|---|---|---|---|---|
| Nanobody | strain | | C121/2020 | | Andina B.1.499 | | WA1/2020 | |
| 10 | negative | | negative | | negative | | negative | |
| 27 | 0.625 | uM | | | 0.3 | uM | | |
| 29 | 1.25 | uM | 0.9 | ug/ml | 10 | uM | >500 | nM |
| 30 | negative | | 0.2 | ug/ml | negative | | 250 | nM |
| 31 | 1.25 | uM | 1.6 | uM | >1 | uM | | |
| 33 | 0.625 | uM | negative | | negative | | >500 | nM |
| 35 | negative | | negative | | negative | | negative | |
| 39A | 0.156 | uM | | | 0.4 | uM | 0.244 | nM |
| 43 | 0.250 | uM | 2.1 | ug/ml | 0.1 | uM | 3.125 | nM |
| 45 | 0.625 | uM | | | 0.4 | uM | | |
| 46 | 0.625 | uM | | | >1 | uM | | |
| 47 | 5 | uM | | | >1 | uM | <500 | nM |
| 48 | 0.078 | uM | 1 | uM | >1 | uM | | |

| | | | | | | |
|---|---|---|---|---|---|---|
| 71 | 1.25 | uM | 1 | uM | 1000 | nM |
| 104 | 0.078 | uM | 0.3 | uM | 0.977 | nM |
| 110 | 0.156 | uM | 2.5 | uM | 15.125 | nM |
| 125 | 0.078 | uM | | | 31.25 | nM |
| 136 | 2.5 | uM | 0.3 | uM | | |
| 145 | 0.078 | uM | 2.5 | uM | 62.5 | nM |

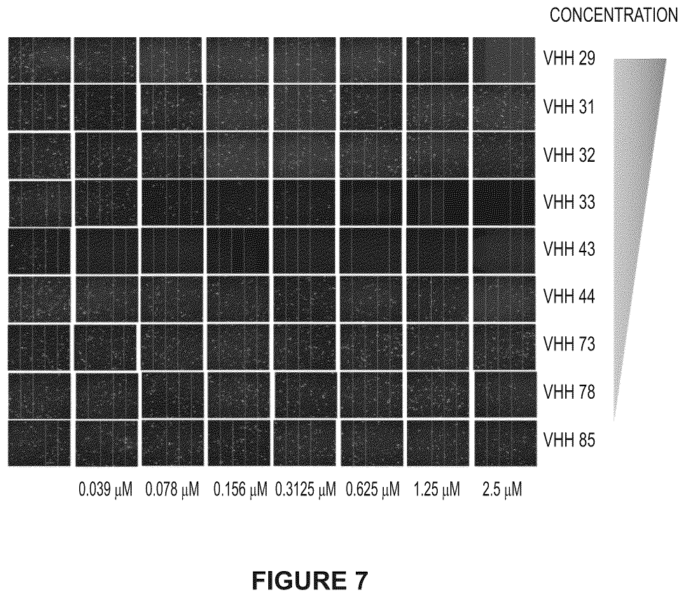

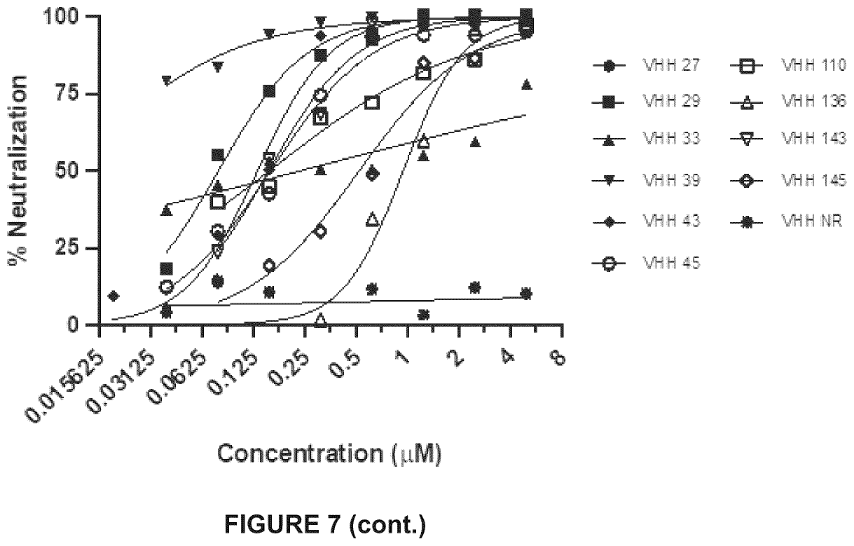

The results summarized in Table 3 show the neutralization capability exhibited by 19 nanobodies of the invention that were able to be tested by at least two of the four VN assays mentioned. Particularly, Nbs 39, 43, 104 and 110 reacting with the RBD domain exhibit significant neutralization activity (see FIG. 7) and were selected for further experiments in mice. Nb 33 and 45, together with other clones directed to epitopes on Spike different to RBD were also selected for test in mice (Example 10) and in an interference assay using biliverdin that have been reported to react with the NTD region (Example 9).

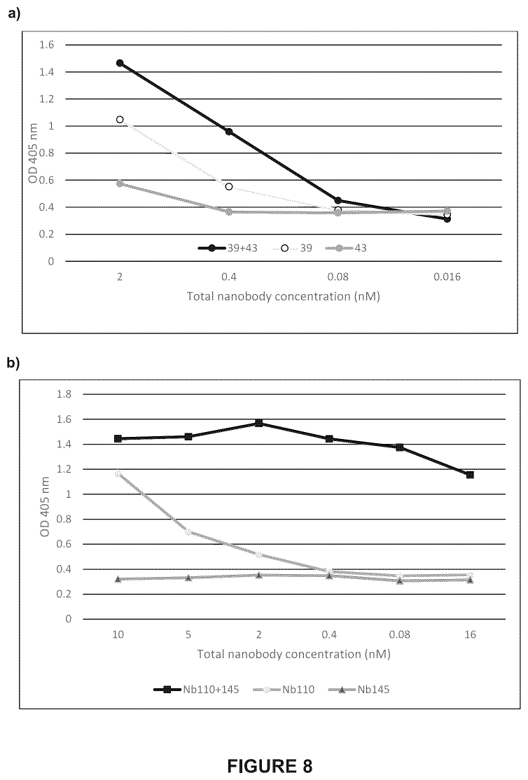

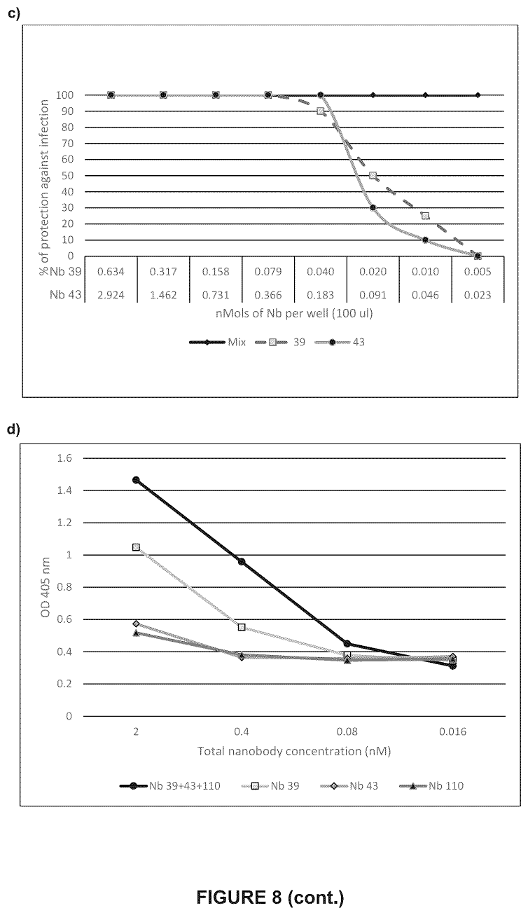

After the results obtained by ELISA and VN, the effects obtained by combinations of different nanobodies were evaluated by both techniques. As depicted in FIG. 8, it can be observed that the equimolar combinations of Nb 39-Nb 43 and Nb 110-Nb 145 produced a synergetic effect. The result was also observed by VN when combining Nb 39 with Nb 43 in 1:3 molar proportion. These results suggest that the molecules could provide an effective cocktail to control COVID-19. The combination of other two molecules or even three also showed this effect (FIG. 8).

### Example 8. Blocking Assay Between ACE2 and RBD

Initially the soluble ACE2 construct (ACE2s) was produced using as template the sequence present in the vector pCAGGS ACE2, obtained from NIH, which contained the complete sequence of the protein including its transmembrane domain. Cloning was performed by PCR using the Fw ACE2 BspEl and Rv ACE2 EcoRV primers that bind to both flanks of the soluble domain of ACE2. After amplifying the expected fragment and making a cut with the restriction enzymes BspEl and EcoRV, a ligation was performed with the vector pCAGGS 2GFP cut with the same enzymes. In this way, a construction was obtained that contains towards the N-terminal end the secretion signal of human serum albumin that allows proteins to be sent to the culture medium after their transfection and towards the C-terminal end a histidine tag to facilitate subsequent purification. The obtained amplicon had a size of 1799 base pairs. Vector pCAGGS, which is used in various constructions, contains the hybrid promoter AG, which is considered a strong promoter that facilitates the expression of proteins. This hybrid promoter is composed of the sequence of the chicken β-actin promoter (first exon and part of the first intron, which acts as an enhancer or enhancer), linked to a fragment of the rabbit β-globin promoter (consisting of a 3 ′part of the second intron and a 5′ part of the third exon). The gel band of expected size was then cut and purified. The insert ligation was performed in the aforementioned vector and then, said construction was transformed into E. coli DH5α bacteria. Subsequently, plasmid DNA was purified and a digestion with the BgIII enzyme was performed to corroborate the pattern of fragments expected after digestion. Positive clones were transfected into HEK-293T eukaryotic cells and purified by IMAC and dialysis.

In a second step vector pCAGGs ACE2s-HRP, which codes for the protein ACE2s coupled to horseradish peroxidase (HRP) was used. Addgene plasmid ACE2 (pcDNA3-sACE2 (WT) -8his, catalog 149268), containing the HA signal peptide (MKTIIALSYIFCLVFA) at its N-terminus was used as template. The plasmid was cut with the enzyme BamHI. On the other hand, the coding sequence for HRP was obtained from the pUC57-anti ASIC-HRP vector, available in the laboratory, which was cut with the enzymes Notl and Smal. This strategy makes it possible to incorporate a histidine tag after the HRP sequence, which facilitates a subsequent purification of the protein using a nickel resin. Both in the case of the vector and the fragment, the Klenow reaction was carried out to generate blunt ends that were compatible at the time of ligation. The vector was further treated with the enzyme CIP (intestinal calf phosphatase) to remove phosphates and avoid re-ligation. The proposed strategy was previously carried out virtually using SnapGene, to corroborate that the coding sequences of ACE2s and HRP were in the correct frame. Both fragments were purified from agarose gel using the Wizard® SV Gel and PCR Clean-Up System kit according to the manufacturer's instructions, for their subsequent ligation. Positive clones were transfected into HEK-293T eukaryotic cells and purified by IMAC and dialysis. The coding sequence of the RBD was obtained from BEI Resources NR-52309. The construct has the signal sequence of the Spike protein at the N-terminal end, followed by the RBD sequence (amino acids 319 to 541) and a histidine tag at the C-terminal end. The sequence was codon-optimized for mammalian expression, subcloned into the aforementioned pCAGGS expression vector and expressed in eukaryotic HEK-293T cells and then purified by IMAC.

The assay was standardized coating ELISA plates with 50 ul per well of 0.5 ug of RBD and, after a blocking step, adding nanobody samples to the plate in serial 2-fold dilutions. Then, the ACE2-HRP was added, and the assay developed with TMB, plates were read at 450 nm. The samples that interfered the interaction of RBD and ACE2 reduced the OD of the blank (no sample).

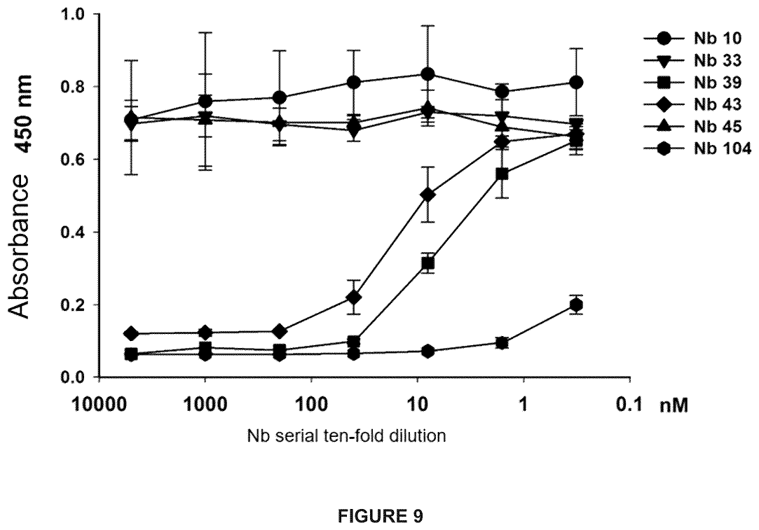

FIG. 9 shows that the nanobodies directed to RBD with strong VN activity, clones 39, 43, and 104, show interference confirming that the neutralizing effect is due to the interference of the RBD domain binding to the cellular receptor. On the other hand, Nb 33 and 45, not directed to RBD, as well as Nb 10 which has being retrieved from a library against bovine coronavirus Mebus and is directed to RBD but does not have VN activity, were not able to block the reaction.

### Example 9. Interference of Biliverdin Binding to Spike

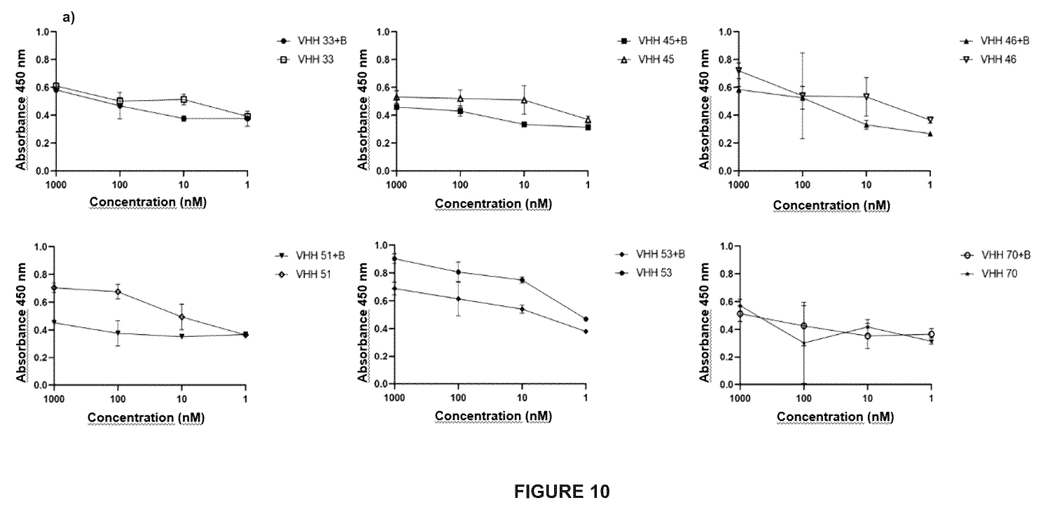

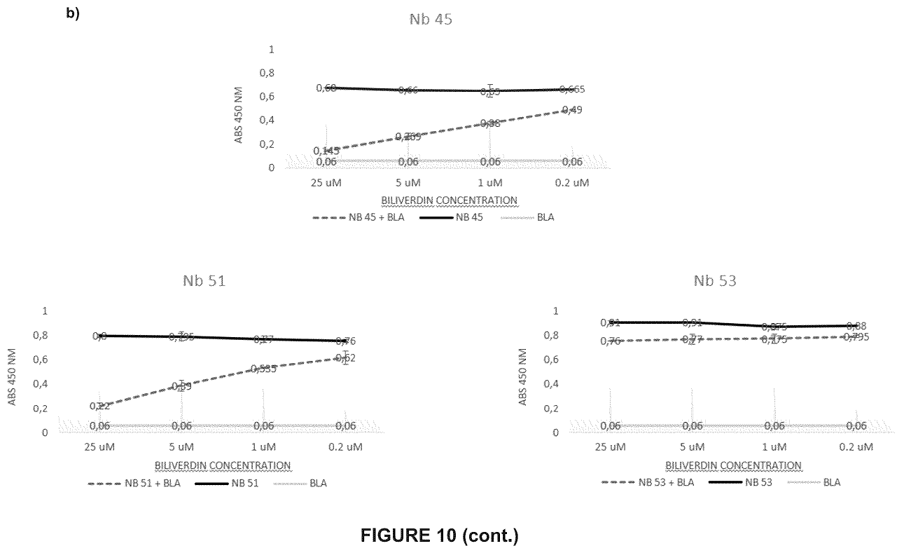

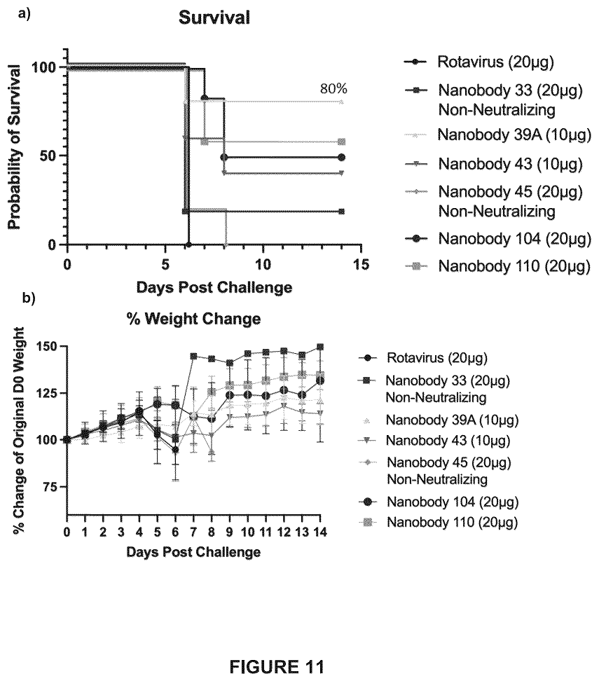

10 nanobodies were selected that recognize the Spike protein, but not RBD. Of these, 6 were tested in an ELISA experiment (in duplicates) in which 96-well plates were sensibilized with 0.05 μg/ml of purified Spike protein. The plates were blocked with 1% skim milk in PBST 0.5% and subsequently 25 μl of 10 μM Biliverdin was added. Then 25 μl of 10th nanoantibody dilutions were added starting from an initial concentration of 1 μM. After using specific antibodies, a detection antibody coupled to HRP was added and the reaction was developed with TMB substrate. Optical density was read at 450 nm. It was observed that the presence of biliverdin impaired the binding of the Nbs 45, 51 and 53 (FIG. 10 (a)).

In a second experiment, a fixed concentration (0.1 μM) of the nanobodies was mixed with serial dilution of biliverdin. Once again, biliverdin blocked the binding of nanobodies 45 and 51 to Spike in a dose-dependent manner (FIG. 10 (b)).

These results suggest that these two nanobodies are directed to an epitope in the NTD region.

### Example 10. Efficacy in a Mouse Model

To assess the protective efficacy of the nanobodies against SARS-COV-2 infection, 4-week-old k18-hACE2 mice (Jackson Labs) were separated into seven groups of eight with equal numbers of males and females in each group. Approximately four hours before challenge, mice were administered either 20 micrograms of a rotavirus control nanobody, and SARS-COV-2 nanobodies 33, 45, 104, and 110. Nanobodies 39A and 43 were administered at a ten-microgram dose. Mice were challenged intranasally with 1×10⁵ PFU of the WA1/2020 strain of SARS-COV-2 in each nostril. Mice were then monitored daily for weight loss and survival, with checks increasing to at least 3 times daily when disease symptoms presented. Four days post challenge, three mice in each group (1 male, 2 females, excluded from weight and survival data) were euthanized, and tissues (i.e., brain, lungs, nasal turbinates, and blood) were collected to assess the impact of the nanobody treatment on viral titers by RT-qPCR.

As it can be seen in FIG. 12, 80% of the mice treated with 10 ug of the clone 39A were protected from dying due to COVID-19. These results are in concordance with the high VN titer of this nanobody observed in the neutralizing test conducted in vitro. The animal receiving 20 ug of Nb 110 and 104 were protected in 60% and 50%, respectively and animals receiving 10 ug of nanobody 43 were 40% protected. Only one animal survived in the group treated with Nb 33, and as expected for a Nb45—a non-neutralizing clone—as well as for the animals receiving the non-related nanobody, all died between days 5 and 10 post challenge (FIG. 12). Regarding the weight gain, it is important to remark that all the survival animals receiving the nanobodies retained or increased their weight over the experiment.

### Example 11. Epitope Mapping of Nanobodies Directed to Linear Epitopes

From nanobodies Nb 10, 35, 39, 104, 110 and 125 that by Western Blot analysis were recognizing linear epitopes on Spike (FIG. 5.C), the inventors tried to identify said epitopes using a peptide array from B.E.I resources (NR-52402). The array was composed by 181-peptides covering the entire amino acid sequence of the Spike (S) glycoprotein of the USA-WA1/2020 strain of SARS-CoV-2 (GenPept: QHO60594). Peptides were 17- or 13-mers, with 10 amino acid overlaps.

To run the assay, the peptides were resuspended in 500 ul of DMSO: PBS to obtain a 2 mg/ml stock. Maxisorp-NUNC 96-well ELISA plates were coated with 100 ng of each peptide (50 μl of a dilution containing 20 μg/ml of each peptide) in coating buffer, pH: 9.6, and incubated overnight at 4° C. After 2 washing with PBST and a blocking step using StartingBlock PBS (Thermo Scientific) for 1 h at room temperature, the nanobodies were added in a concentration of 100 ng per well for 2 h at room temperature, followed by a rabbit anti-VHH (dilution: 1/7000) and a goat anti-Rabbit IgG (Jackson Immuno research) HRP-labeled (dilution: 1/5000). The assay was developed using ABTS substrate and the plates read at 405 nm. As a positive control, capture wells were coated with Spike and RBD. A non-related nanobody directed to rotavirus was used as a negative control.

As preliminary results the assay confirmed that Nb 39 recognized a peptide between amino acids 407-423 in the RBD domain. This result agrees with its neutralizing effect. Nbs 43, 10 and 125 reacted with the same peptide in the region between amino acids 582 and 598 in the carboxy terminal domain 1, CTD 1, of the S1 subunit of Spike, a region characterized for possessing several linear epitopes that induce responses in sera from infected subjects.

Nb 104 bound to two peptides that share the residues IA within the S2 subunit. Nb 33 also bound to one peptide within the S2 domain. Nb 110 reacted with a peptide between amino acids 106 and 122, localized in the NTD region. These results might suggest that the nanobodies in general are not only recognizing these lineal regions of the protein but also might be reacting with other conformational epitopes as well in RBD, since 104 and 110 were selected as recognizing RBD by ELISA. Further crystallographic and structural experiments would be needed to fully address these issues. But in summary the selected nanobodies react with a broad of different epitopes that support their use in a cocktail or combination composition to control COVID-19.

| | Bound | | |
|---|---|---|---|
| Nano- | Pep- | OD | Amino acid sequence |
| body | tide | 405 nm | of the peptide |
| Nb 39 | 59 | 0.423 | 407-VRQIAPGQTGKIADYNY-423 |
| Nb 43 | 84 | 0.555 | 582-LEILDITPCSFGGVSVI-598 |
| Nb 10 | 84 | 0.226 | 582-LEILDITPCSFGGVSVI-598 |
| Nb 125 | 84 | 0.214 | 582-LEILDITPCSFGGVSVI-598 |
| Nb 104 | 125 | 0.731 | 869-MIAQYTSALLAGTITSG-885 |
| | 175 | 0.503 | 1219-GFIAGLIAIVMVTIMLC-1235 |
| Nb 110 | 16 | 0.640 | 106-FGTTLDSKTQSLLIVNN-122 |
| Nb 33 | 161 | 0.375 | 1121-FVSGNCDVVIGIVNNTV-1137 |

In addition to the above embodiments, the present invention may have other embodiments. All technical solutions formed by adopting equivalent substitutions or equivalent transformations fall within the protection scope of the claims of the present invention.

Nanobodies to SARS CoV-2.
DNA and Amino acid Sequence SEQ ID listing

| | SEQ | |
|---|---|---|
| | ID | |
| NAME | NO | SEQUENCE |
| | | DNA |
| NbCoV_10 | 1 | ATGGCCCAGGTGCAGCTGCAGGAGTCTGGGGGAGGCTTGGTGCAG |
| | | GCTGGGGGGTCTCTACACCTCTCCTGTACAGCCTCTGGCTCTGAGA |
| | | ACATGTTGCGTTGGTATGCCGTCGGCTGGTACCGCCAGGCTCCAGG |
| | | GAAGCAGCGCGAGTTGGTCGCAACTATTACTCGTGGTGGTAGTACA |
| | | AACGTTGTAGACTCCGTGAAGGGCCGATTCACCATCTCCAGAGATA |
| | | ATGCCAAGAATATGGTGGCGCTGCAAATGAACAGCCTGAAACCTGA |
| | | GGACACAGCCGTCTATTCCTGTGCAGCAGCGTATGCGCCTTATACT |
| | | GACTATCATCCCGTAGAATATGTCTACTGGGGCCAGGGGACCCAGG |
| | | TCACCGTCTCCTCA |
| NbCoV_25 | 2 | CAGGTGCAGCTGCAGGAGTCTGGGGGAGGATTGGTGCAGGCTGGG |
| | | GACTCTCTGAGACTCTCCTGTGCAGCCTCTGGACGCACCTTCGATA |
| | | CCCATGACATAGGCTGGTTCCGCCAGGCTCCAGGGAAGGAGCGTG |
| | | AGTTTGTAGCAGCTATGGCTTGGAGTGGTGGTAGCACATACTATGCA |
| | | GACTCTGTGAAGGGCCGATTCACCATCTCCAGAGACAGCGCCAAGA |
| | | ACACGGTCAATCTTCAAATGAACAGCCTGAAACCTGAGGACACGGC |
| | | CGTTTATTACTGTGCATCAGAAGCGTTCCAGTCCTCTAGGCTGTATA |
| | | GCGACTATCGTCGGTATCACTACTGGGGCCAGGGGACCCAGGTCA |
| | | CCGTCTCCTCA |
| NbCoV_27 | 3 | CAGGTGCAGCTGCAGGAGTCTGGAGGAGGCTTGGTGCAGCCTGGG |
| | | GGGTCTCTGAGACTCTCCTGTGCAGCCTCTGGAAGCGGCTTCAGTC |
| | | TCAGAGTCATGGGCTGGTACCGCCAGGCTCCAGGGAAGGAGCACC |
| | | GCTTGGTCGCAACTATTAGTAGGACTGGTATCACAAATTATGCAGAC |
| | | TCCGTGAAGGGCCGATTCACCATCTCCAGAGACAACGCCAAGGACA |
| | | CGGTGTATCTGCAAATGAACAACCTGAAACCTGAAGACACGGACGT |
| | | CTATTACTGCTATGCACGGAATTTCGTAGGCGATGACTTTAATCTCT |
| | | GGGGCCAGGGGACCCAGGTCACCGTCTCCTCA |
| NbCoV_28 | 4 | CAGGTGCAGCTGCAGGAGTCTGGGGGAGGCTTGGTGCAGCCTGGG |
| | | GGGTCTCTGAGACTCTCCTGTGCAGCCTCTGGAAGTATCTCCGATAT |
| | | GAGAGCCATGAACTGGTACCGCCAGCCTCCAGGGAAGGAGCGCGA |
| | | GTTGGTCGCGCAAATATTTCTCACTGGTACTATTTACTATGCGGACT |
| | | CCGTGAAGGGCCGATTTACCATCTCCAAGGACAACGCCAAGAACAC |
| | | GGTATATCTGCAAATGAACAACCTGAAACTGGAGGACACGGCCGTC |
| | | TATAGCTGTAGTGCAGACCACTTCCTCGATCAGTTTGATTCTTGGGG |
| | | TCAGGGGACCCAGGTCACCGTCTCCTCA |
| NbCoV_29 | 5 | CAGGTGCAGCTGCAGGAGTCTGGAGGAGGATTGGTGCAGGCTGGG |
| | | GGCTCTCTGAGACTCTCCTGTGCAGCCTCTGGACGCACCTTGTACG |
| | | CCATGGGCTGGTTCCGCCAGGCTCCAGGGAAGGAGCGTGAGTTTG |
| | | TAGCAGCTAAAAGCTGGAATGGTGGTAACACATACTATGCAGACTCC |
| | | GTGAAGGGCCGATTCACCATCTCCAAAGACAACGCCAAGAACACGG |
| | | TGTATCTGCAAATGAACAGCCTGAAACCTGAGGACACGGCCGTTTAT |
| | | AATTGTGCAGCTTCGTCCTGGGACTCGCGTGGTAGTAGTAATTACGT |
| | | TCATTATGACTACTGGGGCCAGGGGACCCAGGTCACCGTCTCCTCA |
| NbCoV_30 | 6 | CAGGTGCAGCTGCAGGAGTCTGGAGGAGGCTTGGTACAGCCTGGG |
| | | GGGTCTCTGAGACTCTCCTGTGCAGCCTCTACCCGCATCATCTTCG |
| | | GTGTCAATGCCATGGGCTGGTACCGCCAGGCTCCAGGGAAGGAGC |
| | | GCGAGTTGGTCGCACGTATTGAGGATGGTGGTACCACAAACTATGC |
| | | AGACTCCGTGAAGGGCCGATTCACCATCTCCAGAGACAACGCCAAG |
| | | AACACGGTGTACCTGCAAATGAACAGCCTGAAACCTGAGGACACGG |
| | | GCATCTATTACTGCAATTACCACAACTCTTACTACGGCTCGGACTAC |
| | | TGGGGCAAAGGGACCCAGGTCACCGTCTCCTCA |
| NbCoV_31 | 7 | CAGGTGCAGCTGCAGGAGTCTGGGGGAGGATTGGTGCAGGCTGGG |
| | | GGCTCTCTGAGACTCTCCTGTGCAGCCTCTGGACGCACCTTCAGTA |
| | | GATCCGCCGTGGCCTGGTTCCGCCAGGCTCCAGGGAAGGAGCGTG |
| | | AATTTGTCGCAGGTATTAGCTGGAGTGGTCGTAACGTAAACTATGCA |
| | | AACTCCGTGAAGGGCCGATTCACCATCTCCAGAGACAGTACCAAGA |
| | | ACACGGTGTATCTGCAAATGAACAGCCTGAAGCCTGAGGACACGGC |
| | | CGTTTATTACTGTGCAACCAGCCAATTCGGGAGTGGTACCAGCGAC |
| | | TCGGAACAGTATGACTACTGGGGCCAGGGGACCCAGGTCACCGTCT |
| | | CCTCA |
| NbCoV_32 | 8 | CAGGTGCAGCTGCAGGAGTCTGGAGGAGGGTCGGTGCAGCCTGGG |
| | | GGGTCTCTGAGACTCTCCTGTGCAGTCTCTAGAAGCCGCTTCAGTC |
| | | TCGTGACCATGGGCTGGTACCGCCAGGCTCCAGGGAAGGAGCGCG |
| | | AATTGGTCGCGGCTATCGCAATTGGTGGCAGCACTAAATATGCAGA |
| | | CTCCGTGAAGGGCCGGTTCACCATCTCCAGAGACAACGCCAAGAGT |
| | | ACGGTGTATCTTCAAATGAACAGTCTACAGCCTGAGGACACGGCCG |
| | | TCTATTATTGTAACGAAGCGCGAGTGTACGACAGTAACTGGTACCCA |
| | | AGGGACTATTGGGGCCAGGGGACCCAGGTCACCGTCTCCTCA |
| NbCoV_33 | 9 | CAGGTGCAGCTGCAGGAGTCTGGAGGAGGATTGGTGCAGGCTGGG |
| | | GGCTCTCTGAGACTCTCCTGTGCAGCCTCTGGACGCACCAGCGATA |
| | | TCTATGCCATGGCCTGGTTCCGCCAGACTCCAGGGAAGGACCGTGA |
| | | GTTTGTAGCAGCTATTACATGGAATGATGGTCTCACATACTATGCAG |
| | | GCTCCGTGAGGGGCCGATTCACCATCTCCAGAGATTACGCCAAGAA |
| | | CACGGTGTATCTACAAATGAACAACCTGAAACCTGAGGACACGGCC |
| | | GTTTATTACGGTGCAGTGACACGCCGTAGATTTGTTACACTCCGCGT |
| | | CCCTGATGACTATGACAAGTGGGGCCAGGGGACCCAGGTCACCGT |
| | | CTCCTCA |
| NbCoV_34 | 10 | CAGGTGCAGCTGCAGGAGTCTGGGGGAGGATTGGTGCAGGCTGGA |
| | | GACTCTCTGAGACTCTCCTGCGCAGCCTCTGGATTCCCCTTCAGTA |
| | | GCTCGGCCATGGGCTGGTTCCGCCAGCCACCAGGAAAGGAGCGTG |
| | | AGTTTGTAACAAGTATTAACTGGAGTGGTGTTCATACACGCCATGCA |
| | | GACTCCGTGAAGGGCCGATTCACCATCTCCAGAGACAACGCCAAGA |
| | | ACACGGTGTATCTGCAAATGAACAGCCTGAAACCTGAGGACACGGC |
| | | CGTTTATTACTGTGCAACAGATCCGGGAAACTTTGGACTGCCTAACA |
| | | GTGAATTAGAGTTTAACTACTGGGGCCAGGGGACCCAGGTCACCGT |
| | | CTCCTCA |
| NbCoV_35 | 11 | CAGGTGCAGCTGCAGGAGTCTGGAGGAGGCTTGGTGCAACCTGGG |
| | | AGGTCTCTGAGACTCTCCTGTGCAGCCTCTGGAANTATCTTCAGTAG |
| | | CTATACCATCATGGGCTGGTTCCGCCAGGCTCCAGGGAAGGAGCGT |
| | | GAGTTTGTAGCATCCATTAGCTGGAGTGGCGGTAACATATACTATGC |
| | | AGACTCCGTGAAGGGCCGATTCACCGTCTCCAGAGACGACGCCAAG |
| | | AACACGGTGTATCTGGATATGAACAATCCGAAACCTGAGGACACGG |
| | | CCGTTTATTACTGTGCATCAGACCTCCGAATGTACGAAGGTGACTGG |
| | | TACGGAGGCTACTGGGGCCAGGGGACCCAGGTCACCGTCTCCTCA |
| NbCoV_36 | 12 | CAGGTGCAGCTGCAGGAGTCTGGGGGAGGATTGGTGCAGGCTGGG |
| | | GGCTCTCTGAGACTCTCCTGTGCAGCCTCTGAACGCACCTCTGGAA |
| | | TGCATGCCACGGCCTGGTTCCGCCAGGCTCCAGGGAAGGAGCGTG |
| | | AGTTTGTAGCACGAATTTTCCACAGTGGTCCTTACACATACTATGCA |
| | | GACTCCGTGAAGGGCCGATTCACCATCTTCAGAGACAGCGCCAAGA |
| | | ACACGGTGGATCTGCAAATGAACAGCCTGAAATCTGAGGACACGGC |
| | | CGTTTATTACTGTGCAGTCGCTCGGTCATGGAGCGATCTGGACTTC |
| | | GTAGACGGGTATGACTACTGGGGTCAGGGGACCCAGGTCACCGTC |
| | | TCCTCA |
| NbCoV_37 | 13 | CAGGTGCAGCTGCAGGAGTCTGGGGGAGGACTGGTGCAGCCTGGG |
| | | GGGTCTCTGAGACTCTCCTGTGCAGCCTCTACAAGCGTCTTCGGTG |
| | | TCAATGACATGGGCTGGTACCGCCAGGCTCCAGAAAAGGAGCGCG |
| | | AGTTGGTCGCACGTATATCGGATGGTGGTACCACAAACTATGCAGA |
| | | CTCCGTGAAGGGCCGATTCACCATCTCCAGAGACAACGCCAAGAAC |
| | | ACGGTGTATCTGCAAATGAACAACCTGAAAGCTGAGGACACGGCCA |
| | | TCTATTACTGCAATTACCACAACTATTACTACGGCCTGGACTTCTGG |
| | | GGCAAAGGCACCCAGGTCACCGTCTCCTCA |
| NbCoV_39 | 14 | CAGGTGCAGCTGCAGGAGTCTGGAGGAGGATTGGTGCAGGCTGGG |
| | | GGCTCTCTGAGACTCTCCTGTGCAGCCTCTGGACGCACCTTCAGTA |
| | | GCACTGCCATGGCCTGGTTCCGCCAGGCTCCAGGACAGGAGCGTG |
| | | AGTTTGTAGCAACTATTAGCTGGAGTGGTGATAACACAAAATATGCA |
| | | GACTCCGTGAAGGGCCGATTCACCATCTCCAGAGACAACGCCAAGA |
| | | ACACGGTGTATCTGCAAATGAACAGCCTGAAACCTGAGGACACGGC |
| | | CGTTTATTACTGTGCAGCAGACCCCGGGAACTTTGGGGTACCCATC |
| | | GAGTATTTTATGTTTGACTACTGGGGCCAGGGGACCCAGGTCACCG |
| | | TCTCCTCA |
| NbCoV_43 | 15 | CAGGTGCAGCTGCAGGAGTCTGGGGGAGGATTGGTGCAGCCTGGG |
| | | GGGTCTCTGAGACTCTCCTGTGTAGCCTCTGGAAGCATCGGTAGTA |
| | | TCAACCACATGGCCTGGTTTCGCAAGCCTCCAGGGAAGGAGCGCGA |
| | | GTTGGTCGCATCAATTACTCTGGCTCCGGATGAAATCGCCTACTATG |
| | | CAGACTCCGTGAAGGGCCGATTCGCCATCTCCAAAGGCAACGCCCA |
| | | GCCCACCTTGTATCTGCAAATGAACAGCCTGAAACCTGAGGACACG |
| | | GCCGTCTACTATTGTCATTTTCGTCAGATCCGTGCGAACGCACATGA |
| | | TGTCTACTGGGGCCAGGGGACCCAGGTCACCGTCTCCTCA |
| NbCoV_44 | 16 | CAGGTGCAGCTGCAGGAGTCTGGAGGAGGATTGGTGCAGGCTGGG |
| | | GGCTCTCTGAGACTCTCCTGTGCAGCCTCTGGACGCACCGTCAGTT |
| | | ATCGTGTGATGGGCTGGTTCCGCCAGGCTCCAGGGAAGGAGCGTG |
| | | AGTTTGTAGCGTTCGTGGACTGGAGTTCAGGTACTGCATACTATGCA |
| | | GAGTCCGTGCAGGGCCGATTCATCATGTCCAGAGACACTGCCGACA |
| | | ACACGGGGTTTCTGCAAATGAACAACCTGAAACCTGAGGACACGGC |
| | | CGTTTATTACTGCGCAGCAGATCCTTTTTTTCGAGTCATCGCTTATAG |
| | | CGACCCTAAACGCTATAGCTACTGGGGCCAGGGGACCCAGGTCAC |
| | | CGTCTCCTCA |
| NbCoV_45 | 17 | CAGGTGCAGCTGCAGGAGTCTGGAGGAGGATTGGTGCAGGCTGGG |
| | | GGCTCTCTGAGACTCTCCTGTGTAGCCTCCGGACGCACCTTGTACG |
| | | CCATGGGCTGGTTCCGCCAGGCTCCAGGGAAGGAGCGTGAATTTGT |
| | | AGCAGCTACTAGCTGGAATGGTGGTAATACTAACTATGGAGAGTCC |
| | | GTGAAGGGCCGATTCACCATCTCCAAAGACAACGCCAAGAACACGG |
| | | TGTATCTGCAAATGAACAGCCTGAAACCTGAAGACACGGCCGTTTAT |
| | | AATTGTGCAGCTTCGTCCCGGGACTCGCGTGGTACAAGTACTTACG |
| | | CCAGTTATGACTATTGGGGCCAGGGGACCCAGGTCACCGTCTCCTC |
| | | A |
| NbCoV_46 | 18 | CAGGTGCAGCTGCAGGAGTCTGGGGGAGGATTGGTGCAGGCTGGG |
| | | GACTCTCTGAGACTCTCCTGTGCAGCCTCTGGACGCACCTTCAGTA |
| | | GCCTTTCCGCGGGCTGGTTCCGCCAGGCTCCAGGGAAGGAGCGTG |
| | | AGTTTGTAGCAGCTATTAGGTGGAGTGATTATAACACATACTATGTA |
| | | GACTCCGTGAAGGGCCGATTCACCATCTCCAGAGACAACGCCAAGA |
| | | ACACGGTGTATCTGCAAATGGACAGCCTGAAACCCGAGGACACGGC |
| | | CGTTTATTACTGTGCAGCCACGGCATTGGGATACCGGTACGCCTCC |
| | | CTGGAGTCGCGGAATGAGTATCGCTATTGGGGCCAGGGGACCCAG |
| | | GTCACCGTCTCCTCA |
| NbCoV_47 | 19 | CAGGTGCAGCTGCAGGAGTCTGGAGGAGGATTGGTGCAGGCTGGG |
| | | GGCTCTCTGAGACTCTCCTGTGCAGCCTCTGGACGCACCTCCGATA |
| | | TCTATGCCATGGCCTGGTTCCGCCAGGCTCCAGGGAAGGACCGTGA |
| | | GTTTGTAGCTGCTATTACATGGAATAATGGTGAAGTATACTATAGCAA |
| | | CTCCGTGAAGGGCCGATTCACCATCTCCAGAGATTACGCCAAGAAC |
| | | ACGGTGTATCTACAAATGAACAGCCTAGAACCTGAGGACACGGCCG |
| | | TTTATTACGGTGCAGCCACACGCCGTAGATACCATACACTCCGCGTC |
| | | TCTGGTGATTATGACAACTGGGGCCAGGGGACCCAGGTCACCGTCT |
| | | CCTCA |
| NbCoV_48 | 20 | CAGGTGCAGCTGCAGGAGTCTGGAGGAGGCTTGGTGCAGCCTGGG |
| | | GGGTCTCTGAGACTCTCCTGTGTAGCCTCCAGAAGCATCGGCAGTA |
| | | TCAACCACATGGCCTGGTATCGCAAGGCTCCAGGGAAGGAGCGCG |
| | | AGTTGGTCGCGTCAATTACTCTTAGTCCCAGTGAGACCGTGTACTAT |
| | | ACAGAAGTCGTGAAGGGCCGATTTGCCATCTCCAAAGGGAACGCCC |
| | | AGAATACCTTGTATCTGCAAATGAACAGTCTGAAACCTGAGGACACG |
| | | GCCGTCTACTATTGTCATTTTCGTCAGATTCGTGCGATCGCGGATGA |
| | | TGTCTACTGGGGCCAGGGGACCCAGGTCACCGTCTCCTCA |
| NbCoV_49 | 21 | CAGGTGCAGCTGCAGGAGTCTGGAGGAGGCTTGGTGCAGCCTGGG |
| | | GGTTCTCTGAGTCTCTCCTGTACAACTTCTGGAGAGTACCACACTAT |
| | | TAATTACATGGGATGGTACCGCCAGGCTCCAGGGAAGGAGCGCGA |
| | | GTTAGTCGCACGTATTAGTTTCGGTGGTCGCATAGAATATGGAGACT |
| | | TCGTGAAGGGCCGATTCACCCTCTCCAGAAACAACGCCAAGAACAC |
| | | GGTGAATCTACAAATGAACAGCCTGAAGCCTGAGGACACGGCCGTC |
| | | TATTACTGTAATGGAGAAATTACTACACTGATTAGTGTCTTAAGTACC |
| | | GACCAGTACTGGGGCCAGGGGACCCAGGTCACCGTCTCCTCA |
| NbCoV_50 | 22 | CAGGTGCAGCTGCAGGAGTCTGGGGGAGGCTTGGTGCAGCCTGGG |
| | | GGGTCTCTGAGACTCTCCTGTGCAGCCTCTGGAAGTATCTCCGATAT |
| | | GAGAGCCATGAACTGGTACCGCCAGCCTCCAGGAAAGGAGAGCGA |
| | | GCTGGTCGCGCAAATATTTCTCACTGGTACGATTTACTATCGGGACT |
| | | CCGTGAAGGGCCGATTTACCATCTCCAAGGACAACGCCAAGAACAC |
| | | GGTATATCTGCAAATGAACAACCTGAAACTGGAGGACACGGCCGTC |
| | | TATATCTGTAATGCAGACCACTTCCTCGACCAGTTTGATTCTTGGGG |
| | | CCAGGGGACCCAGGTCACCGTCTCCTCA |
| NbCoV_51 | 23 | CAGGTGCAGCTGCAGGAGTCTGGAGGAGGATTGGTGCAGGCTGGG |
| | | GGCTCGCTGAGACTCTCCTGTGTAACCTCTGGATGTACCTTCACTAC |
| | | GTATGCCATGGGCTGGTTCCGCCAGGCTCCAGGGAAGGAGCGTGA |
| | | ATTTGTAGCAGCAGTTACCTGGAGTGGTGGTAGCACATATTATGCAG |
| | | ATTCCGTGAAGGGCCGATTCACCATCTCCAGAGACAACGGCAAGAA |
| | | TACGGTGTATTTGCAGATGAACAGCCTGAAACATGGGGACACGGCC |
| | | GTTTATTACTGTGCAGCACGATATCCCGGAAAGTGGGGTGAACTGC |
| | | CTACGGATACTCGGTTTGACTTCTGGGGCCAGGGGACCCAGGTCAC |
| | | CGTCTCCTCA |
| NbCoV_53 | 24 | CAGGTGCAGCTGCAGGAGTCTGGAGGAGGATTGGTGCCGACTGGG |
| | | GGCTCTCTGAGACTCTCCTGTGCAGCCTCTGGACGCGACTTCGAGT |
| | | CCTATGCCATGGGCTGGTTCCGCCAGGCTCCAGGGAAGGAGCGTG |
| | | AGTTTGTAGCAGCTAGTAGGTGGATTGGTGGTCCCACATACTATGCA |
| | | AACTCCGTGAAGGCCCGATTCACCTTCTCCGGAGACAACGCCAAGC |
| | | GCACGGTATATCTGCAAATGAACAGCCTGAAACCTGAGGACACGGC |
| | | CGTTTATTACTGTGCAGCGGGGGGACGAATATCTAATGAGGGCTTC |
| | | AAGCCTGACTACTGGGGCCAGGGGACCCAGGTCACCGTCTCCTCA |
| NbCoV_54 | 25 | CAGGTGCAGCTGCAGGAGTCTGGGGGAGGATTGGTGCAGGCTGGG |
| | | GGCTCTCTGAGACTCTCCTGTGCAGCCTCTGGAGTCACCTTCAGTA |
| | | GATATGCCATGGCCTGGTTCCGCCAGGCTCCAGGGAAGGAGCGTG |
| | | AATTTGTCGCAGGTAATAGTTGGAGCGGTCGTAACGTAAACTATGCA |
| | | GACTCCGTGAAGGGCCGATTCACCATCACCAGAGACAACGCCAAGA |
| | | ACACGGTGTATCTGCAAATGAATAGCCTGAAACCTGAGGACACGGC |
| | | CGTTTATTACTGTGCAGCGAGCCAATTCGGGAGTGGTGCCGACTAC |
| | | TCGGAACAATATGACTACTGGGGCCAGGGGACCCAGGTCACCGTCT |
| | | CCTCA |
| NbCoV_55 | 26 | CAGGTGCAGCTGCAGGAGTCTGGGGGAGGATTGGTGCAGGCTGGG |
| | | GGCTCTCTGAGGCTCTCCTGTGCAGCCTCTGGACGCACCTTGTACG |
| | | CCATGGGCTGGTTCCGTCAGGCTCCAGGGAAGGAGCGTGAGTTTGT |
| | | AGCAGCTACAAGCTGGAATGGTGGTAACACATACCTTCCAGACTCC |
| | | GTGAAGGGCCGATTCACCATCTCCAGAGACAACGCCAAGAACACGG |
| | | TATATCTGCAAATGAACAGCCTGAAACCTGAGGACACGGCCGTTTAT |
| | | GTTTGTGCAGCTTCGTTCCGGGACTCGCGTGGTAGTAGTAATTACG |
| | | CCCATTATGACTACTGGGGCCAGGGGACCCAGGTCACCGTCTCCTC |
| | | A |
| NbCoV_56 | 27 | CAGGTGCAGCTGCAGGAGTCTGGAGGAGGATTGGTACAGACTGGG |
| | | GGCTCTCTGAGACTCTCCTGTGCAGCCTCTGGACGCACCTTCAGTA |
| | | CCTATAATATGGGCTGGTTCCGCCAGGCTCCAGGGAAGGAGCGTGA |
| | | GTTTGTAGCACTGATTAACTGGAGTGGTGGTGACACATACTATACAA |
| | | ACTCCGTGAAGGGCCGATTCACCATCTCCAGAGACAACGCCAAGAA |
| | | CACGTTGTATCTGCAAATGAACAACCTGAAACCTGAGGACACGGCC |
| | | GTTTATTCTTGTGCGGCGCGATATGCGGCCATTGGTAGTCCCTACTG |
| | | GAAGCTTACGACTCCGAGTGAGTACGAATACTGGGGCCAGGGGAC |
| | | CCAGGTCACCGTCTCCTCA |
| NbCoV_59 | 28 | CAGGTGCAGCTGCAGGAGTCTGGGGAGGATTGGTGCAGGCTGGGG |
| | | CCTCTCTCAGACTCTCCTGTGCAGCCTCTGGACGCACCTTCAGTGG |
| | | CTATTACATGGGCTGGTTCCGCCAGGCTCCAGGGAAGGAGCGTGAA |
| | | TTTGTAGCATTTATTAGGTGGAGTGGTGGTGAGACATACTATACAGA |
| | | CTCCGTGAAGGGCCGATTCACCATCTCCAGAGACAACACCAAGAAC |
| | | ACGATGTATCTGCAAATGAACAGTTTGAAACCTGAGGACACGGCCG |
| | | TTTATTACTGTGCAGCAGATAAGGGGATGGGTTACTATCGCGACGCT |
| | | GCCCTCGCATCCTGGTATGACTACTGGGGCCAGGGGACCCAGGTC |
| | | ACCGTCTCCTCA |
| NbCoV_70 | 29 | CAGGTGCAGCTGCAGGAGTCTGGAGGAGGGTTGGTGGAGGCTGGG |
| | | GGCTCTCTGAGACTCTCCTGTGCAGCCTCTGGACGCACCAGCGATA |
| | | TCTATGCCATGGCCTGGTTCCGCCAGGCTCCAGGGCAGGACCGTG |
| | | AGTTTGTAGCAGCTATTACATGGAATGATGGTATCACATACTATTCAT |
| | | CCTCCGTGAAGGGCCGATTCACCATCTCCAGAGATTACGCCAAGAA |
| | | CACGGTGTATCTACAAATGAACAGCCTGAGTCCTGAGGACACGGCC |
| | | GTTTATTACGGTGCAGTCACACGCCGTAGATACGTTACACTCCGCGT |
| | | CGCTGATGACTATGACAAATGGGGCCAGGGGACCCAGGTCACCGT |
| | | CTCCTCA |
| NbCoV_71 | 30 | CAGGTGCAGCTGCAGGAGTCTGGGGGAGGGTTGGTGCAGGCTGGG |
| | | GGCTCTCTGAGACTCTCCTGTGCAGCCTCTGGACTGACCTTTGGAT |
| | | ACACTGCCACGGCCTGGTTCCGCCAGGCTCCAGGGAAGGAGCGTG |
| | | AGTTTGTTGCGCGAATTTTCAAAAGAGGTGGCTACACATACTATTCA |
| | | GACTCCGTGAAGGGCCGATTCACCGTCTCCAGAGACAGCGCCAAG |
| | | GACACGGTGTATCTGCAAATGAACAGCCTGAAATCTGAGGACACGG |
| | | CCGTTTATTACTGTGCAGTCGCTCGGGAATGGAGCGATCTGGACTTT |
| | | CGAGACGGCTATGACTACTGGGGTCAGGGGACCCAGGTCACCGTC |
| | | TCCTCA |
| NbCoV_74 | 31 | CAGGTGCAGCTGCAGGAGTCTGGGGGAGGATTGGTGCAGGCTGGG |
| | | GGCTCGCTGAGACTCTCCTGTGCAGCCTCTGGACGCACCTTCACTA |
| | | CCTATGTCATGGGCTGGTTCCGCCAGGCTCCAGGGAAGGAGCGTG |
| | | AGTTTGTAGCAGCTGTTTCCTGGAATGGTGGTAGCACATACTATGCA |
| | | GATTCCGTGAAGGGCCGATTCACCATCTCCAGAGACAACGGCGAGA |
| | | ACACGGTGTATTTGCAGATGAACACCCTGAAACATGGGGACACGGC |
| | | CGTTTATTACTGTGCAGCACGATATCCCGGAAAGTGGGGTGAACTG |
| | | CCTACGGATACTCGGTTTGACTTTTGGGGCCAGGGGACCCAGGTCA |
| | | CCGTCTCCTCA |
| NbCoV_78 | 32 | CAGGTGCAGCTGCAGGAGTCTGGAGGAGGATTGGTGCAGGCTGGG |
| | | GGCTCTCTGAGACTCTCCTGTGTAGCCTCTGGACGCGCCTTCAGTA |
| | | GCAGTTCTATGGGCTGGTTCCGCCAGGCTCCCGGGAAGGAGCGTG |
| | | AGTTTGTAGCAGCTATTAGGTGGAGTGATGATAATACAGTCTATGTA |
| | | GACTCCGTGAAGGGCCGATTCGCCATCTCCAGAGACAACGCCAAGA |
| | | ACACGGTGTATCTGCAAATGAACAGCCTGAAACCCGAGGACACGGC |
| | | CGTTTATTACTGCGCAGCCACGGGATTGGGGTACCGGTACGCCGTC |
| | | CTGGAGTCGCGATCTGAGTATCGCTACTGGGGCCAGGGGACCCAG |
| | | GTCACCGTCTCCTCA |
| NbCoV_89 | 33 | CAGGTGCAGCTGCAGGAGTCTGGGGGAGGCTTGGTGCAACCTGGG |
| | | GGGTCTCTGAGACTCTCCTGTGCAGCCTCGGCAAGCATCGTCAGTT |
| | | TCAATGCCATTGGCTGGCACCGCCAGGCTCCAGGGAAGGAGCGCG |
| | | AGTTGGTCGCAAGTATCTCCAGTGGTGGTGTCGCAAACTATGTAGA |
| | | CTCCGTGAAGGGCCGATTCACCATCTCCAGAGACAACGCCAAGAAC |
| | | ACGGTGTATCTGCAAATGAACAGCCTGAAACCTGAGGACACGGCCG |
| | | TTTATCACTGTGCAGCAAAGACCAGGGGGGTGTACGGCACTAACTG |
| | | GCTCTCCCCAGTCGACTATGAATATGGCTCCTGGGGCCAGGGGACC |
| | | CAGGTCACCGTCTCCTCA |
| NbCoV_96 | 34 | CAGGTGCAGCTGCAGGAGTCTGGGGGAGGCTTGGTGCAGCCTGGG |
| | | GGGTCTCTGAGACTCTCCTGTGCAGCCTCTGGAAGTATCTCTAATAT |
| | | GAGAGCCATGAACTGGTACCGCCAGCAGCCAGGGAAGGAGCGCGA |
| | | GTTGGTCGCGCAAATATTTCTCACTGGTACAATTTACATTCGGGACT |
| | | CCGTGAAGGGCCGGTTTACCATCTCCAAGGACAACGCCAAGAACAT |
| | | GGTATATCTGCAAATGAACAACCTGAAACTGGAGGACACGGCCGTC |
| | | TATATCTGCAGTGCAGACCACTTCCTCGACCAGTTTGATTCTTGGGG |
| | | CCAGGGGACCCAGGTCACCGTCTCCTCA |
| NbCoV_100 | 35 | CAGGTGCAGCTGCAGGAGTCTGGAGGAGGATTGGTGCAGGCTGGG |
| | | GGCTCTCTGAGACTCTCCTGTGCAGCCTCTGGACGCACCTTCAGTA |
| | | GATCCGCCGTGGCCTGGTTCCGCCAGGCTCCAGGGAAGGAGCGTG |
| | | AATTTGTCGCAGGTATTAGCTGGAGTGGTCGTAACGTAAACTATGCA |
| | | AACTCCGTGAAGGGCCGATTCACCATCTCCAGAGACAATACCAAGA |
| | | ACACGGTGTATCTGCAAATGAACAGCCTGAAGCCTGAGGACACGGC |
| | | CGTTTATTACTGTGCAACCAGCCAATTCGGGAGTGGTACCAGCGAC |
| | | TCGAAACAGTATGACTACTGGGGCCAGGGGACCCAGGTCACCGTCT |
| | | CCTCA |
| NbCoV_103 | 36 | CAGGTGCAGCTGCAGGAGTCTGGGGGAGGCTTGGTGCAGCCTGGG |
| | | GGGTCTCTGAGACTCTCCTGTGCAGCCTCTGGAAGTATCTCCGATAT |
| | | GAGAGCCATGGACTGGTACCGCCAGCCTCCAGGGAAGGAGCGCGA |
| | | GTTGGTCGCGCAAATATTTCTCACTGGTACTACTTACTATTTGGACTC |
| | | CGTGAAGGGCCGATTTACCATCTCCAAGGACAACGCCAAGAACACG |
| | | GTATATCTGCAAATGAACAACCTGAAAGTGGAGGACACGGCCGTCT |
| | | ATATCTGTAATGCAGACCACTTCCTCGACCAGTTTGATTCTTGGGGC |
| | | CAGGGGACCCAGGTCACCGTCTCCTCA |
| NbCoV_104 | 37 | CAGGTGCAGCTGCAGGAGTCTGGGGGAGGCTTGGTGCAGCCTGGG |
| | | GGGTCTCTGAGACTCTCCTGTGCAGCCTCTGGAAGCATCTTCCGAG |
| | | TCATGGGCTGGTACCGCCAGGCTCCAGGAAAGGAGCACGACTTGG |
| | | TCGCAACTATTACAAGGTCTGGTGACACAAACTACGCAGACTCCGTG |
| | | AAGGGCCGATTCACCATCTCCAGAGACAACACCAAGAATACGGTGT |
| | | ATTTACAAATGAACAACCTGAAACCTGAGGACACGGACGTCTATTAC |
| | | TGCTATGCACGGAATTTTGTAGGACATAATCCGGACGTCTGGGGCC |
| | | AGGGGACCCAGGTCACCGTCTCCTCA |
| NbCoV_105 | 38 | CAGGTGCAGCTGCAGGAGTCTGGAGGAGGATTGGTGCAGGCTGGG |
| | | GGCTCTCTGAGACTCGACTGTGCAGCCTCTGGATTGACCTTCAGCA |
| | | AGTATACCATGGGGTGGTTCCGCCAGGCTCCAGGGAAGGAGCGGG |
| | | AATTTGTTGCAGTTATTTCGGGGAATGATGATATCACATACTATACAG |
| | | ACTCCGTGAAGGGCCGATTCACCATCTCCAGAGACAATGGCAAGAA |
| | | CACGGTGTATTTGCAAATGAACAGCCTGAAACCTGAGGACACGGCC |
| | | GTATATTACTGTGCAGCAGATCGGGGTGAGAGTTACTACTACAGCC |
| | | GTCCGTCTGAGTATACCTATTGGGGCCAGGGGACCCAGGTCACCGT |
| | | CTCCTCA |
| NbCoV_106 | 39 | CAGGTGCAGCTGCAGGAGTCTGGAGGAGGCCTGGTGCAGCCTGGG |
| | | GGGTCTCTGAGACTCTCCTGTGCAGCCTCTGGAAGCATCTTCCGAG |
| | | TCATGGGCTGGTACCGCCAGGCTCCAGGAAAGGAGCACGAGTTGG |
| | | TCGCAACTATTACAAAGTCTGGTGACACAAACTACGCAGACTCCGTG |
| | | AAGGGCCGATTCACCATCTCCAGAGACAACGCCAAGGATACGGTGT |
| | | ATTTGCAAATGAACAACCTGAAACCTGAGGACACGGACGTCTATTAC |
| | | TGCTATGCACGGAATTTTGTAGGCCGTAATCCGGACGTCTGGGGCC |
| | | AGGGGACCCAGGTCACCGTCTCCTCA |
| NbCoV_107 | 40 | CAGGTGCAGCTGCAGGAGTCTGGAGGAGGATTGGTGCAGGCTGGG |
| | | GGCTCTCTGAGACTCTCCTGTGCAGCCTCTGGACGCACCTTCAGTA |
| | | GCACCGCCATGGGCTGGTTCCGCCAGGCTCCAGGGAAGGAGCGTG |
| | | AATTTGTAGCAGTTATTAGCTGGAGTGGTGCTAACACACACTATTCA |
| | | GACTCCGTGAAGGGCCGATTCAGCATCTCCAGAGACAACGCCAAGA |
| | | ACACGGTGTATCTGCAAATGAACAGCCTGAAACCTGAGGACACGGC |
| | | CGTTTATTACTGCGCAGCAGACCCCGGGAACTTTGGGGTACCCGTC |
| | | GAGCATTTTTTGTATGACTTTTGGGGCCAGGGGACCCAGGTCACCG |
| | | TCTCCTCA |
| NbCoV_108 | 41 | CAGGTGCAGCTGCAGGAGTCTGGGGGAGGAGTGGTGCAGGCTGG |
| | | GGACTCTCTGAGACTCTCCTGTGCAGTTTCTGGACGCACCGGCAGA |
| | | ATGGCCTGGTTCCGCCAGGCTCCAGGGAAGGAGCGTGAGTTTGTA |
| | | GCAGGTATTATGCGGACTGGAAGCTTCACAAATTATATAGGTTCCGT |
| | | GAAGGGCCGATTCACCATCTCCATGGACAACGCCAAGAACACAGTG |
| | | TATCTGCAAATGAACAGCCTGAAAGTTGAGGACACGGCCGTTTATTA |
| | | CTGTGCAGCCACGGGAGACGGTGGTTCGTATGACTACTGGGGCCA |
| | | GGGGACCCAGGTCACCGTCTCCTCA |
| NbCoV_110 | 42 | CAGGTGCAGCTGCAGGAGTCTGGGGGAGGATTGGTGCAGGCTGGG |
| | | GACTCTCTGAGACTCTCCTGTGCAGCCTCTGGACGCACCTTTGGAA |
| | | GCCACGCCACGGCCTGGTTCCGCCAGGCTCCGGGGAAGGAGCGTG |
| | | AGTTTGTTGCACGAATTTTCTGGAGGGGTGATTATACATACTATGCA |
| | | GACTCCGTGAAGGGCCGATTCACCATCTCCAGAGACAGCGCCAAGA |
| | | ACACGGTGTATCTTCAAATGAACAGCCTGAAATCTGAGGACACGGC |
| | | CGTTTATTACTGTGCAGTCGCTCGGTCATGGAGCGATCTGGATTTTG |
| | | AAAACGGGTATGATTACTGGGGTCAGGGGACCCAGGTCACCGTCTC |
| | | CTCA |
| NbCoV_121 | 43 | CAGGTGCAGCTGCAGGAGTCTGGAGGAGGCTTGGTGCAGCCTGGG |
| | | GGGTCTCTGAGACTCTCCTGTGTAGCCTCTGGAAGTATCTCCAATAT |
| | | GAGAGCCATGAACTGGTACCGCCAGCCTCCAGGGAAGGAGCGCGA |
| | | GTTGGTCGCGCAAATATTTCTCACTGGTACTATTTACTATCGGGACT |
| | | CCGTGAAGGGCCGATTTACCATCTCCAAGGACAACGCCAAGAACAC |
| | | GGTATATCTGCAAATGAACAACCTGAAACTGGAGGACACGGCCGTC |
| | | TATAGTTGTAGTGCAGACCACTTCCTCGACCAGTTTGATTCTTGGGG |
| | | CCAGGGGACCCAGGTCACCGTCTCCTCA |
| NbCoV_123 | 44 | CAGGTGCAGCTGCAGGAGTCTGGGGGAGGCTTGGTGCAGCCTGGG |
| | | GGGTCTCTGAGACTCTCCTGTGTAGCCTCTGGAAGTATCTCCAATAT |
| | | GAGAGCCATGAACTGGTACCGCCAGCCTCCAGGGAAGGAGCGCGA |
| | | GTTGGTCGCGCAAATATTTCTCACTGGTACTATTTACTATCGGGACT |
| | | CCGTGAAGGGCCGATTTACCATCTCCAAGGACAACGCCAAGAACAC |
| | | GGTATATCTGCAAATGAACAACCTGAAACTGGAGGACACGGCCGTC |
| | | TATAGTTGTAGTGCAGACCACTTCCTCGACCAGTTTGATTCTTGGGG |
| | | CCAGGGGACCCAGGTCACCGTCTCCTCA |
| NbCoV_125 | 45 | CAGGTGCAGCTGCAGGAGTCTGGAGGAGGATTGGTGCAGCCTGGG |
| | | GGGTCTCTGAGACTCTCCTGTGCAGCCTCTGCACACTTCTTCAGTG |
| | | GCGGCGCCATGGGCTGGTACCGCCAGGCTCCAGGGAAGGAGCGC |
| | | GAGTTGGTCGCAACAATTACTGGTGGCGGGGACACCAACTATGCTG |
| | | AGCACGTGAAGGGCCGATGCACCATCTCCAGCGACATATCCGACAA |
| | | CACGGTGTATCTGCAGATGATCAGCCTCAAACCTGATGACACGGCC |
| | | GTCTATTACTGTTATGCACGCGACCTGCTTCAGCATCCCTTCTGGGG |
| | | CCAGGGGACCCAGGTCACCGTCTCCTCA |
| NbCoV_134 | 46 | CAGGTGCAGCTGCAGGAGTCTGGGGGAGGCTGGGTGCGGCCTGG |
| | | GGGGTCTCTGAGACTCTCCTGTGCAGCCTCTGGAAGTATGTTCAGT |
| | | CTCAGAGTCATGGGTTGGTACCGCCAGGCTCCAGGGAAGGAGCAC |
| | | GACTTGGTCGCAACTATTAGTAGGACTGGTATGACAAACTATGCAGA |
| | | CTCCGTGAAGGGCCGATTCACCATCCCAGAGACAACGCCAAGAACA |
| | | CGGTGTATCTGCAAATGAACAACCTGAAACCTGAGGACACGGACGT |
| | | CTATTACTGCTATGCACGGAATTTCGTTGGCGATGACTTTGATCTCT |
| | | GGGGCCAGGGGACCCAGGTCACCGTCTCCTCA |
| NbCoV_136 | 47 | CAGGTGCAGCTGCAGGAGTCTGGAGGAGGATTGGTGCAGACTGGG |
| | | GGCTCTCTGAGACTCTCCTGTGCAGCCTCTGGACGCACCGCCAGTA |
| | | GTTATACCATGGGCTGGTTCCGCCAGGCTGCGGGGAAGGAGCGGG |
| | | ACTTTGTAGCAGCAATACGCGAGAGTCTTGGATATACAAACTATGCA |
| | | GACTCCGTGAAGGGCCGATTCACCATCTCCAGAGACAACGCCAACA |
| | | ACACGGTGTATCTGCAAATGAACAGCCTGAAACCTGAGGACACGGC |
| | | CGTTTATTACTGTGCAGCCTATGAAGGTATTCGTTACTCCAGCACGA |
| | | GGGCTGATTTTTATAACTACTGGGGTCAGGGGACCCAGGTCACCGT |
| | | CTCCTCA |
| NbCoV_137 | 48 | CAGGTGCAGCTGCAGGAGTCTGGGGGAGGATTGGTGCAGGCTGGG |
| | | GGCTCTCTGAGGCTCTCCTGCGCAGCCTCTGGATTTACCTTCGGTA |
| | | GCTCGGCCATGGGCTGGTTCCGCCAGCCTCCAGGAAAGGAGCGTG |
| | | AGTTTGTAGCAAGTATTAACTGGAGTGGTGTTCATACACGCCATGCA |
| | | GACTCCGTGAAGGGCCGATTCACCATCTCCAGAGACAACGCCAAGA |
| | | ACACGGTGTATCTGCAAATGAACAGCCTGAAACCTGAGGACACGGC |
| | | CGTTTATTACTGTGCAACAGATCCGGGAAACTTTGGACTGCCTAACA |
| | | GTGAATTAGAGTTTAACTACTGGGGCCAGGGGACCCAGGTCACCGT |
| | | CTCCTCA |
| NbCoV_138 | 49 | CAGGTGCAGCTGCAGGAGTCTGGGGGAGGATTGGTGCAGGCTGGG |
| | | GGCTCTCTGAGACTCTCCTGTGCAGCCTCTGGACGCACCGTCAGTT |
| | | TGCGTGTGATGGGCTGGTTCCGCCAGGCTCCAGGGAAGGAGCGTG |
| | | AGTTTGTAGCGTTTGTGGACTGGAATTCAGGTAATGCATACTATTCA |
| | | GACTCCGTGCAGGGCCGATTCATCATGTCCAGAGACAACGCCGAGA |
| | | ACACGGCGTTTCTGCAAATGAACAGCCTGAAACCTGAGGACACGGC |
| | | CGTTTATTACTGCGCAGCAGATCCTTTTTTTCGAGTCATCGCTTATAG |
| | | CGACCCTAAACGCTATAGCTACTGGGGCCAGGGGACCCAGGTCAC |
| | | CGTCTCCTCA |
| NbCoV_139 | 50 | CAGGTGCAGCTGCAGGAGTCTGGGGGAGGATTGGTGCGGGCTGGG |
| | | GGCTCTCTGAGACTCTCCTGTGCAGCCTCTGGACGCACCTTGGGTA |
| | | TTTATCGTATGAGCTGGTTCCGCCAGGCTCCAGGGAAGGAACGTGA |
| | | ATTTGTAGCAGCTGTTATGTGGAGTGGTCTACACCCAGTCTATGCAG |
| | | ACTCTGTGAAAGGCCGATTCACCATCTCCGGAGACAACGCCAAGAT |
| | | GACGCTATATCTACAAATGAACAACCTGACTTCTGAGGACACGGCCA |
| | | CTTATTACTGTGCAGCCCGACAGCCGACCACTGCGACGTATGACTA |
| | | CTGGGGCCAGGGGACCCAGGTCACCGTCTCCTCA |
| NbCoV_143 | 51 | CAGGTGCAGCTGCAGGAGTCTGGAGGAGGCTTGGTGCAGCCTGGG |
| | | GGGTCTCTGAGACTCTCCTGTGCAGCCTCTGGAAGCATCTTCAGCC |
| | | TCAGAGTCATGGGCTGGTACCGCCAAACTCCAGGAAAGGAGCACGA |
| | | CTTGGTCGCAATTATTAGTAGGTCTGGTAACGCAAATTACGCAGACT |
| | | CCGTGAAGGGCCGATTCACCATCTCCAGAGACAACGCCAAAAATAC |
| | | GGTGTATCTGCAAATGAACAACCTGAAACCTGAAGACACGGACGTC |
| | | TATTACTGCTATGCACGGAATTTTATAGGCGATAATCCGGACGTCTG |
| | | GGGCCAGGGGACCCAGGTCACCGTCTCCTCA |
| NbCoV_145 | 52 | CAGGTGCAGCTGCAGGAGTCTGGGGGAGGATTGGTGCAGGCTGGG |
| | | GGCTCTCTGAGACTCTCCTGTGCAGCCTCTGGACGCACCTTCAGTA |
| | | GCTATGCCATGGGCTGGTTCCGCCAGGCTCCAGGGAAGGAGCGTG |
| | | AGTTTGTAGCAGTGATTAACTGGAGTGGTGCTAACACACGCTATACA |
| | | GACTCCGTGAAGGGCCGATTCAGCATCTCCAGAGACAACGCCAAGA |
| | | ACACGGTGTATCTGCAAATGAACAGCCTGAAACCTGAGGACACGGC |
| | | CGTTTATTACTGTGCAGCAGACCCCGGGAACTTTGGGGTACCCATC |
| | | GAGCATTTTATGTATGACTACTGGGGCCAGGGGACCCAGGTCACCG |
| | | TCTCCTCA |
| NbCoV_152 | 53 | CAGGTGCAGCTGCAGGAGTCTGGAGGAGGCTTGGTGCAGCCTGGG |
| | | GGGTCTCTGAGACTCTCCTGTACAGCCTCTGGAAGCATCTTCCGAG |
| | | TCATGGGCTGGTACCGCCTGGCTCCAGGAAAGGATTACGACTTGGT |
| | | CGCAACTATTACAGGGTCTGGTGACACAAACTACGCAGAGTCCGTG |
| | | AAGGGCCGATTCACCATCTCCAGAGACAACGCCAAGAATACGGTGT |
| | | ATTTGCAAATGAACAACCTGAGACCTGAGGACACGGACGTCTATTAC |
| | | TGCTATGCACGGAATTTTGTAGGACGTAATCCGGACGTCTGGGGCC |
| | | AGGGGACCCAGGTCACCGTCTCCTCA |
| | | PRT |
| NbCoV_10 | 54 | QVQLQESGGGLVQAGGSLHLSCTASGSENMLRWYAVGWYRQAPGKQ |
| | | RELVATITRGGSTNVVDSVKGRFTISRDNAKNMVALQMNSLKPEDTAVY |
| | | SCAAAYAPYTDYHPVEYVYWGQGTQVTVSS |
| NbCoV_25 | 55 | QVQLQESGGGLVQAGDSLRLSCAASGRTFDTHDIGWFRQAPGKEREF |
| | | VAAMAWSGGSTYYADSVKGRFTISRDSAKNTVNLQMNSLKPEDTAVYY |
| | | CASEAFQSSRLYSDYRRYHYWGQGTQVTVSS |
| NbCoV_27 | 56 | QVQLQESGGGLVQPGGSLRLSCAASGSGFSLRVMGWYRQAPGKEHR |
| | | LVATISRTGITNYADSVKGRFTISRDNAKDTVYLQMNNLKPEDTDVYYC |
| | | YARNFVGDDFNLWGQGTQVTVSS |
| NbCoV_28 | 57 | QVQLQESGGGLVQPGGSLRLSCAASGSISDMRAMNWYRQPPGKERE |
| | | LVAQIFLTGTIYYADSVKGRFTISKDNAKNTVYLQMNNLKLEDTAVYSCS |
| | | ADHFLDQFDSWGQGTQVTVSS |
| NbCoV_29 | 58 | QVQLQESGGGLVQAGGSLRLSCAASGRTLYAMGWFRQAPGKEREFV |
| | | AAKSWNGGNTYYADSVKGRFTISKDNAKNTVYLQMNSLKPEDTAVYNC |
| | | AASSWDSRGSSNYVHYDYWGQGTQVTVSS |
| NbCoV_30 | 59 | QVQLQESGGGLVQPGGSLRLSCAASTRIIFGVNAMGWYRQAPGKERE |
| | | LVARIEDGGTTNYADSVKGRFTISRDNAKNTVYLQMNSLKPEDTGIYYC |
| | | NYHNSYYGSDYWGKGTQVTVSS |
| NbCoV_31 | 60 | QVQLQESGGGLVQAGGSLRLSCAASGRTFSRSAVAWFRQAPGKEREF |
| | | VAGISWSGRNVNYANSVKGRFTISRDSTKNTVYLQMNSLKPEDTAVYY |
| | | CATSQFGSGTSDSEQYDYWGQGTQVTVSS |
| NbCoV_32 | 61 | QVQLQESGGGSVQPGGSLRLSCAVSRSRFSLVTMGWYRQAPGKERE |
| | | LVAAIAIGGSTKYADSVKGRFTISRDNAKSTVYLQMNSLQPEDTAVYYC |
| | | NEARVYDSNWYPRDYWGQGTQVTVSS |
| NbCoV_33 | 62 | QVQLQESGGGLVQAGGSLRLSCAASGRTSDIYAMAWFRQTPGKDREF |
| | | VAAITWNDGLTYYAGSVRGRFTISRDYAKNTVYLQMNNLKPEDTAVYY |
| | | GAVTRRRFVTLRVPDDYDKWGQGTQVTVSS |
| NbCoV_34 | 63 | QVQLQESGGGLVQAGDSLRLSCAASGFPFSSSAMGWFRQPPGKERE |
| | | FVTSINWSGVHTRHADSVKGRFTISRDNAKNTVYLQMNSLKPEDTAVY |
| | | YCATDPGNFGLPNSELEFNYWGQGTQVTVSS |
| NbCoV_35 | 64 | QVQLQESGGGLVQPGRSLRLSCAASGXIFSSYTIMGWFRQAPGKEREF |
| | | VASISWSGGNIYYADSVKGRFTVSRDDAKNTVYLDMNNPKPEDTAVYY |
| | | CASDLRMYEGDWYGGYWGQGTQVTVSS |
| NbCoV_36 | 65 | QVQLQESGGGLVQAGGSLRLSCAASERTSGMHATAWFRQAPGKERE |
| | | FVARIFHSGPYTYYADSVKGRFTIFRDSAKNTVDLQMNSLKSEDTAVYY |
| | | CAVARSWSDLDFVDGYDYWGQGTQVTVSS |
| NbCoV_37 | 66 | QVQLQESGGGLVQPGGSLRLSCAASTSVFGVNDMGWYRQAPEKERE |
| | | LVARISDGGTTNYADSVKGRFTISRDNAKNTVYLQMNNLKAEDTAIYYC |
| | | NYHNYYYGLDFWGKGTQVTVSS |
| NbCoV_39 | 67 | QVQLQESGGGLVQAGGSLRLSCAASGRTFSSTAMAWFRQAPGQERE |
| | | FVATISWSGDNTKYADSVKGRFTISRDNAKNTVYLQMNSLKPEDTAVYY |
| | | CAADPGNFGVPIEYFMFDYWGQGTQVTVSS |
| NbCoV_43 | 68 | QVQLQESGGGLVQPGGSLRLSCVASGSIGSINHMAWFRKPPGKEREL |
| | | VASITLAPDEIAYYADSVKGRFAISKGNAQPTLYLQMNSLKPEDTAVYYC |
| | | HFRQIRANAHDVYWGQGTQVTVSS |
| NbCoV_44 | 69 | QVQLQESGGGLVQAGGSLRLSCAASGRTVSYRVMGWFRQAPGKERE |
| | | FVAFVDWSSGTAYYAESVQGRFIMSRDTADNTGFLQMNNLKPEDTAVY |
| | | YCAADPFFRVIAYSDPKRYSYWGQGTQVTVSS |
| NbCoV_45 | 70 | QVQLQESGGGLVQAGGSLRLSCVASGRTLYAMGWFRQAPGKEREFV |
| | | AATSWNGGNTNYGESVKGRFTISKDNAKNTVYLQMNSLKPEDTAVYN |
| | | CAASSRDSRGTSTYASYDYWGQGTQVTVSS |
| NbCoV_46 | 71 | QVQLQESGGGLVQAGDSLRLSCAASGRTFSSLSAGWFRQAPGKEREF |
| | | VAAIRWSDYNTYYVDSVKGRFTISRDNAKNTVYLQMDSLKPEDTAVYY |
| | | CAATALGYRYASLESRNEYRYWGQGTQVTVSS |
| NbCoV_47 | 72 | QVQLQESGGGLVQAGGSLRLSCAASGRTSDIYAMAWFRQAPGKDREF |
| | | VAAITWNNGEVYYSNSVKGRFTISRDYAKNTVYLQMNSLEPEDTAVYY |
| | | GAATRRRYHTLRVSGDYDNWGQGTQVTVSS |
| NbCoV_48 | 73 | QVQLQESGGGLVQPGGSLRLSCVASRSIGSINHMAWYRKAPGKEREL |
| | | VASITLSPSETVYYTEVVKGRFAISKGNAQNTLYLQMNSLKPEDTAVYY |
| | | CHFRQIRAIADDVYWGQGTQVTVSS |
| NbCoV_49 | 74 | QVQLQESGGGLVQPGGSLSLSCTTSGEYHTINYMGWYRQAPGKEREL |
| | | VARISFGGRIEYGDFVKGRFTLSRNNAKNTVNLQMNSLKPEDTAVYYC |
| | | NGEITTLISVLSTDQYWGQGTQVTVSS |
| NbCoV_50 | 75 | QVQLQESGGGLVQPGGSLRLSCAASGSISDMRAMNWYRQPPGKESE |
| | | LVAQIFLTGTIYYRDSVKGRFTISKDNAKNTVYLQMNNLKLEDTAVYICN |
| | | ADHFLDQFDSWGQGTQVTVSS |
| NbCoV_51 | 76 | QVQLQESGGGLVQAGGSLRLSCVTSGCTFTTYAMGWFRQAPGKERE |
| | | FVAAVTWSGGSTYYADSVKGRFTISRDNGKNTVYLQMNSLKHGDTAVY |
| | | YCAARYPGKWGELPTDTRFDFWGQGTQVTVSS |
| NbCoV_53 | 77 | QVQLQESGGGLVPTGGSLRLSCAASGRDFESYAMGWFRQAPGKERE |
| | | FVAASRWIGGPTYYANSVKARFTFSGDNAKRTVYLQMNSLKPEDTAVY |
| | | YCAAGGRISNEGFKPDYWGQGTQVTVSS |
| NbCoV_54 | 78 | QVQLQESGGGLVQAGGSLRLSCAASGVTFSRYAMAWFRQAPGKERE |
| | | FVAGNSWSGRNVNYADSVKGRFTITRDNAKNTVYLQMNSLKPEDTAVY |
| | | YCAASQFGSGADYSEQYDYWGQGTQVTVSS |
| NbCoV_55 | 79 | QVQLQESGGGLVQAGGSLRLSCAASGRTLYAMGWFRQAPGKEREFV |
| | | AATSWNGGNTYLPDSVKGRFTISRDNAKNTVYLQMNSLKPEDTAVYVC |
| | | AASFRDSRGSSNYAHYDYWGQGTQVTVSS |
| NbCoV_56 | 80 | QVQLQESGGGLVQTGGSLRLSCAASGRTFSTYNMGWFRQAPGKERE |
| | | FVALINWSGGDTYYTNSVKGRFTISRDNAKNTLYLQMNNLKPEDTAVYS |
| | | CAARYAAIGSPYWKLTTPSEYEYWGQGTQVTVSS |
| NbCoV_59 | 81 | QVQLQVWGGLVQAGASLRLSCAASGRTFSGYYMGWFRQAPGKEREF |
| | | VAFIRWSGGETYYTDSVKGRFTISRDNTKNTMYLQMNSLKPEDTAVYY |
| | | CAADKGMGYYRDAALASWYDYWGQGTQVTVSS |
| NbCoV_70 | 82 | QVQLQESGGGLVEAGGSLRLSCAASGRTSDIYAMAWFRQAPGQDREF |
| | | VAAITWNDGITYYSSSVKGRFTISRDYAKNTVYLQMNSLSPEDTAVYYG |
| | | AVTRRRYVTLRVADDYDKWGQGTQVTVSS |
| NbCoV_71 | 83 | QVQLQESGGGLVQAGGSLRLSCAASGLTFGYTATAWFRQAPGKEREF |
| | | VARIFKRGGYTYYSDSVKGRFTVSRDSAKDTVYLQMNSLKSEDTAVYY |
| | | CAVAREWSDLDFRDGYDYWGQGTQVTVSS |
| NbCoV_74 | 84 | QVQLQESGGGLVQAGGSLRLSCAASGRTFTTYVMGWFRQAPGKERE |
| | | FVAAVSWNGGSTYYADSVKGRFTISRDNGENTVYLQMNTLKHGDTAV |
| | | YYCAARYPGKWGELPTDTRFDFWGQGTQVTVSS |
| NbCoV_78 | 85 | QVQLQESGGGLVQAGGSLRLSCVASGRAFSSSSMGWFRQAPGKERE |
| | | FVAAIRWSDDNTVYVDSVKGRFAISRDNAKNTVYLQMNSLKPEDTAVY |
| | | YCAATGLGYRYAVLESRSEYRYWGQGTQVTVSS |
| NbCoV_89 | 86 | QVQLQESGGGLVQPGGSLRLSCAASASIVSFNAIGWHRQAPGKERELV |
| | | ASISSGGVANYVDSVKGRFTISRDNAKNTVYLQMNSLKPEDTAVYHCA |
| | | AKTRGVYGTNWLSPVDYEYGSWGQGTQVTVSS |
| NbCoV_96 | 87 | QVQLQESGGGLVQPGGSLRLSCAASGSISNMRAMNWYRQQPGKERE |
| | | LVAQIFLTGTIYIRDSVKGRFTISKDNAKNMVYLQMNNLKLEDTAVYICSA |
| | | DHFLDQFDSWGQGTQVTVSS |
| NbCoV_100 | 88 | QVQLQESGGGLVQAGGSLRLSCAASGRTFSRSAVAWFRQAPGKEREF |
| | | VAGISWSGRNVNYANSVKGRFTISRDNTKNTVYLQMNSLKPEDTAVYY |
| | | CATSQFGSGTSDSKQYDYWGQGTQVTVSS |
| NbCoV_103 | 89 | QVQLQESGGGLVQPGGSLRLSCAASGSISDMRAMDWYRQPPGKERE |
| | | LVAQIFLTGTTYYLDSVKGRFTISKDNAKNTVYLQMNNLKVEDTAVYICN |
| | | ADHFLDQFDSWGQGTQVTVSS |
| NbCoV_104 | 90 | QVQLQESGGGLVQPGGSLRLSCAASGSIFRVMGWYRQAPGKEHDLVA |
| | | TITRSGDTNYADSVKGRFTISRDNTKNTVYLQMNNLKPEDTDVYYCYAR |
| | | NFVGHNPDVWGQGTQVTVSS |
| NbCoV_105 | 91 | QVQLQESGGGLVQAGGSLRLDCAASGLTFSKYTMGWFRQAPGKERE |
| | | FVAVISGNDDITYYTDSVKGRFTISRDNGKNTVYLQMNSLKPEDTAVYY |
| | | CAADRGESYYYSRPSEYTYWGQGTQVTVSS |
| NbCoV_106 | 92 | QVQLQESGGGLVQPGGSLRLSCAASGSIFRVMGWYRQAPGKEHELVA |
| | | TITKSGDTNYADSVKGRFTISRDNAKDTVYLQMNNLKPEDTDVYYCYAR |
| | | NFVGRNPDVWGQGTQVTVSS |
| NbCoV_107 | 93 | QVQLQESGGGLVQAGGSLRLSCAASGRTFSSTAMGWFRQAPGKERE |
| | | FVAVISWSGANTHYSDSVKGRFSISRDNAKNTVYLQMNSLKPEDTAVY |
| | | YCAADPGNFGVPVEHFLYDFWGQGTQVTVSS |
| NbCoV_108 | 94 | QVQLQESGGGVVQAGDSLRLSCAVSGRTGRMAWFRQAPGKEREFVA |
| | | GIMRTGSFTNYIGSVKGRFTISMDNAKNTVYLQMNSLKVEDTAVYYCAA |
| | | TGDGGSYDYWGQGTQVTVSS |
| NbCoV_110 | 95 | QVQLQESGGGLVQAGDSLRLSCAASGRTFGSHATAWFRQAPGKEREF |
| | | VARIFWRGDYTYYADSVKGRFTISRDSAKNTVYLQMNSLKSEDTAVYY |
| | | CAVARSWSDLDFENGYDYWGQGTQVTVSS |
| NbCoV_121 | 96 | QVQLQESGGGLVQPGGSLRLSCVASGSISNMRAMNWYRQPPGKERE |
| | | LVAQIFLTGTIYYRDSVKGRFTISKDNAKNTVYLQMNNLKLEDTAVYSCS |
| | | ADHFLDQFDSWGQGTQVTVSS |
| NbCoV_123 | 97 | QVQLQESGGGLVQPGGSLRLSCVASGSISNMRAMNWYRQPPGKERE |
| | | LVAQIFLTGTIYYRDSVKGRFTISKDNAKNTVYLQMNNLKLEDTAVYSCS |
| | | ADHFLDQFDSWGQGTQVTVSS |
| NbCoV_125 | 98 | QVQLQESGGGLVQPGGSLRLSCAASAHFFSGGAMGWYRQAPGKERE |
| | | LVATITGGGDTNYAEHVKGRCTISSDISDNTVYLQMISLKPDDTAVYYCY |
| | | ARDLLQHPFWGQGTQVTVSS |
| NbCoV_134 | 99 | QVQLQESGGGWVRPGGSLRLSCAASGSMFSLRVMGWYRQAPGKEH |
| | | DLVATISRTGMTNYADSVKGRFTISRDNAKNTVYLQMNNLKPEDTDVYY |
| | | CYARNFVGDDFDLWGQGTQVTVSS |
| NbCoV_136 | 100 | QVQLQESGGGLVQTGGSLRLSCAASGRTASSYTMGWFRQAAGKERD |
| | | FVAAIRESLGYTNYADSVKGRFTISRDNANNTVYLQMNSLKPEDTAVYY |
| | | CAAYEGIRYSSTRADFYNYWGQGTQVTVSS |
| NbCoV_137 | 101 | QVQLQESGGGLVQAGGSLRLSCAASGFTFGSSAMGWFRQPPGKERE |
| | | FVASINWSGVHTRHADSVKGRFTISRDNAKNTVYLQMNSLKPEDTAVY |
| | | YCATDPGNFGLPNSELEFNYWGQGTQVTVSS |
| NbCoV_138 | 102 | QVQLQESGGGLVQAGGSLRLSCAASGRTVSLRVMGWFRQAPGKERE |
| | | FVAFVDWNSGNAYYSDSVQGRFIMSRDNAENTAFLQMNSLKPEDTAV |
| | | YYCAADPFFRVIAYSDPKRYSYWGQGTQVTVSS |
| NbCoV_139 | 103 | QVQLQESGGGLVRAGGSLRLSCAASGRTLGIYRMSWFRQAPGKEREF |
| | | VAAVMWSGLHPVYADSVKGRFTISGDNAKMTLYLQMNNLTSEDTATYY |
| | | CAARQPTTATYDYWGQGTQVTVSS |
| NbCoV_143 | 104 | QVQLQESGGGLVQPGGSLRLSCAASGSIFSLRVMGWYRQTPGKEHDL |
| | | VAIISRSGNANYADSVKGRFTISRDNAKNTVYLQMNNLKPEDTDVYYCY |
| | | ARNFIGDNPDVWGQGTQVTVSS |
| NbCoV_145 | 105 | QVQLQESGGGLVQAGGSLRLSCAASGRTFSSYAMGWFRQAPGKERE |
| | | FVAVINWSGANTRYTDSVKGRFSISRDNAKNTVYLQMNSLKPEDTAVY |
| | | YCAADPGNFGVPIEHFMYDYWGQGTQVTVSS |
| NbCoV_152 | 106 | QVQLQESGGGLVQPGGSLRLSCTASGSIFRVMGWYRLAPGKDYDLVA |
| | | TITGSGDTNYAESVKGRFTISRDNAKNTVYLQMNNLRPEDTDVYYCYA |
| | | RNFVGRNPDVWGQGTQVTVSS |

1. A SARS-COV-2 Spike protein binding molecule comprising at least one immunoglobulin single variable domain, wherein the SARS-COV-2 Spike protein binding molecule is a llama-derived single domain antibody having an amino acid sequence with at least 80% sequence identity to any one of SEQ ID NOs: 54 to 106, more preferably, at least 90% sequence identity to any one of SEQ ID NOs: 54 to 106, even more preferably, at least 99% sequence identity to any one of SEQ ID NOs 54 to 106.

2-4. (canceled)

5. The SARS-COV-2 Spike protein binding molecule of claim 1, wherein the single domain antibody has an amino acid sequence with at least 80% sequence identity to any one of the group consisting of SEQ ID NOs: 67, 68, 90, 95, 98, 100 and 105, more preferably, at least 90% sequence identity to any one of the group consisting of SEQ ID NOs: 67, 68, 90, 95, 98, 100 and 105, even more preferably, at least 99% sequence identity to any one of the group consisting of SEQ ID NOs: 67, 68, 90, 95, 98, 100 and 105.

6. The SARS-COV-2 Spike protein binding molecule of claim 1, wherein the single domain antibody has an amino acid sequence with at least 80% sequence identity to any one of the group consisting of SEQ ID NOs: 67, 68, 90 and 95, more preferably, at least 90% sequence identity to any one of the group consisting of SEQ ID NOs: 67, 68, 90 and 95, even more preferably, at least 99% sequence identity to any one of the group consisting of SEQ ID NOs: 67, 68, 90 and 95.

7. The SARS-COV-2 Spike protein binding molecule of claim 1, wherein the single domain antibody has an amino acid sequence as set forth in any of SEQ ID NO: 54 to SEQ ID NO: 106.

8. The SARS-COV-2 Spike protein binding molecule of claim 5, wherein the single domain antibody has an amino acid sequence as set forth in any of the group consisting of SEQ ID NOs: 67, 68, 90, 95, 98, 100 and 105.

9. The SARS-COV-2 Spike protein binding molecule of claim 6, wherein the single domain antibody has an amino acid sequence as set forth in any of the group consisting of SEQ ID NOs: 67, 68, 90 and 95.

10. The SARS-COV-2 Spike protein binding molecule of claim 9, wherein the single domain antibody has an amino acid sequence as set forth in SEQ ID NO: 67.

11. A nucleic acid molecule encoding a SARS-COV-2 Spike protein binding molecule according to claim 1, wherein the nucleic acid molecule has a nucleotide sequence having 80% or more or 90% or more homology to a nucleotide sequence as set forth in any of SEQ ID NO: 1 to SEQ ID NO: 53.

12. (canceled).

13. The nucleic acid molecule of claim 11, wherein the nucleic acid molecule has a nucleotide sequence having 80% or more or 90% or more homology to a nucleotide sequence selected from the group consisting of SEQ ID NOs: 14, 15, 37, 42, 45, 47, and 52.

14. The nucleic acid molecule of claim 13, wherein the nucleic acid molecule has a nucleotide sequence having 80% or more or 90% or more homology to a nucleotide sequence selected from the group consisting of SEQ ID NOs: 14, 15, 37 and 42.

15. The nucleic acid molecule of claim 11, wherein the nucleic acid molecule has a nucleotide sequence as set forth in any of SEQ ID NO: 1 to SEQ ID NO: 53.

16. The nucleic acid molecule of claim 15, wherein the nucleic acid molecule has a nucleotide sequence selected from the group consisting of SEQ ID NOs: 14, 15, 37, 42, 45, 47, and 52.

17. The nucleic acid molecule of claim 16, wherein the nucleic acid molecule has a nucleotide sequence selected from the group consisting of SEQ ID NOs: 14, 15, 37 and 42.

18. The nucleic acid molecule of claim 17, wherein the nucleic acid molecule has a nucleotide sequence as set forth in SEQ ID NO: 14.

19. A pharmaceutical composition comprising a SARS-COV-2 Spike protein binding molecule according to claim 1 and a pharmaceutical acceptable carrier or excipient.

20. A pharmaceutical composition comprising at least two SARS-COV-2 Spike protein binding molecules according to claim 1 and a pharmaceutical acceptable carrier or excipient.

21. The pharmaceutical composition of claim 20, wherein the pharmaceutical composition comprises a SARS-COV-2 Spike protein binding molecule having the amino acid sequence set forth in SEQ ID NO: 67 and a SARS-COV-2 Spike protein binding molecule having the amino acid sequence set forth in SEQ ID NO: 68.

22. The pharmaceutical composition of claim 20, wherein the pharmaceutical composition comprises a SARS-COV-2 Spike protein binding molecule having the amino acid sequence set forth in SEQ ID NO: 95 and a SARS-COV-2 Spike protein binding molecule having the amino acid sequence set forth in SEQ ID NO: 105.

23. The pharmaceutical composition of claim 20, wherein the pharmaceutical composition comprises three different SARS-COV-2 Spike protein binding molecules.

24. The pharmaceutical composition of claim 23, wherein the pharmaceutical composition comprises a SARS-COV-2 Spike protein binding molecule having the amino acid sequence set forth in SEQ ID NO: 67, a SARS-COV-2 Spike protein binding molecule having the amino acid sequence set forth in SEQ ID NO: 68, and a SARS-COV-2 Spike protein binding molecule having the amino acid sequence set forth in SEQ ID NO: 95.

25-27. (canceled)

28. A method for preventing and/or treating a disease caused by infection by novel coronavirus SARS-COV-2, comprising administering a therapeutically effective amount of a SARS-COV-2 Spike protein binding molecule according to claim 1 to a subject in need thereof.

29. The method of claim 28, wherein preventing and/or treating a disease caused by infection by novel coronavirus SARS-COV-2 comprises inhibiting the infection of a subject by novel coronavirus SARS-COV-2.

30. The method of claim 28, wherein the disease caused by the infection by novel coronavirus SARS-COV-2 is selected from the group consisting of human novel coronavirus pneumonia, intestinal disease, intra vascular disseminated coagulation, and encephalitis.

31-33. (canceled).

34. A method for preparing a SARS-COV-2 Spike protein binding molecule according to claim 1, comprising screening a phage library, wherein screening a phage library comprises:
i. doing a series of biopannings with prefused locked Spike protein and Receptor Binding Domain (RBD); and
ii. conducting phage ELISAs and periplasmic extract ELISAs with target antigens to confirm their recognition by the SARS-COV-2 Spike protein binding molecule.

35. (canceled).

36. The method of claim 34, further comprising:
iii. performing a sequencing analysis on the SARS-COV-2 Spike protein binding molecules identified in item ii.;
iv. transforming expression bacteria with the DNA secuence determined in item iii.; and
v. expressing and purifying the corresponding SARS-COV-2 Spike protein binding molecule.
